SEQUENCE LISTING (173 sequences)

SEQ ID NO: 1 - protein, 28 aa, Rattus norvegicus
  DISULFID 7..23: note = Intrachain disulfide bond
SLRRSSCFGGRIDRIGAQSGLGCNSFRY

SEQ ID NO: 2 - protein, 28 aa, Homo sapiens
  DISULFID 7..23: note = Intrachain disulfide bond
SLRRSSCFGGRMDRIGAQSGLGCNSFRY

SEQ ID NO: 3 - protein, 29 aa, synthetic construct
  SITE 1: note = absent, S or E
  SITE 2: note = absent, L, K, 4-Pal, H or E
  SITE 3: note = absent, R, beta-Ala, P, K, E or G
  SITE 9: note = G, 4-Pal or H
  SITE 10: note = G, K, R or Dap
  SITE 11: note = R, K, G or Dap
  SITE 13: note = D or G
  SITE 17: note = A, H, Dap, K, R or Orn
  SITE 18: note = Q, Y or 4-Pal
  SITE 26: note = F or L
  SITE 28: note = Y, H or 4-Pal
  SITE 29: note = absent, GGP or selected from SEQ ID NO:4-20
  MOD_RES 29: note = C-terminal amino acid is optionally amidated
XXXRSSCFXXXIXRIGXXSGLGCPSXRXX

SEQ ID NO: 4 - protein, 7 aa, synthetic construct
SGAPPPE

SEQ ID NO: 5 - protein, 8 aa, synthetic construct
KITAKEDE

SEQ ID NO: 6 - protein, 10 aa, synthetic construct
GPSSGAPPPE

SEQ ID NO: 7 - protein, 10 aa, synthetic construct
GPSSGAPPPS

SEQ ID NO: 8 - protein, 10 aa, synthetic construct
GGSSGAPPPS

SEQ ID NO: 9 - protein, 11 aa, synthetic construct
GGPSSGAPPPS

SEQ ID NO: 10 - protein, 11 aa, synthetic construct
KGPSSGAPPPS

SEQ ID NO: 11 - protein, 11 aa, synthetic construct
GGKSSGAPPPS

SEQ ID NO: 12 - protein, 11 aa, synthetic construct
  SITE 6: note = Aib
GGPPSXKPPPK

SEQ ID NO: 13 - protein, 11 aa, synthetic construct
GSPSSGAPPPS

SEQ ID NO: 14 - protein, 12 aa, synthetic construct
RITAREDKQGYA

SEQ ID NO: 15 - protein, 12 aa, synthetic construct
RITAREDKQGEA

SEQ ID NO: 16 - protein, 12 aa, synthetic construct
  SITE 12: note = PEG24-G
GSPSSGAPPPSX

SEQ ID NO: 17 - protein, 13 aa, synthetic construct
SGSPSSGAPPPSG

SEQ ID NO: 18 - protein, 13 aa, synthetic construct
GGESSGEPPPSEE

SEQ ID NO: 19 - protein, 14 aa, synthetic construct
GSGSPSSGAPPPSG

SEQ ID NO: 20 - protein, 16 aa, synthetic construct
SGSPSSGAPPPSEEEG

SEQ ID NO: 21 - protein, 29 aa, synthetic construct
  MOD_RES 1: note = N-terminal modified with Fatty acid-Z1-Z2-Z3-; wherein Fatty acid is a C16-C26 fatty acid, Z1 comprises an amino acid selected from gamma-Glu, E and beta-Ala, Z2 is either absent or comprises a four to ten amino acid sequence comprising amino acids independently selected from E, K, G, P, A and S, and Z3 is either absent or comprises a polyethylene glycol or a (2-[2-(2-amino-ethoxy)-ethoxy]-acetyl) moiety
  SITE 1: note = absent, S or E
  SITE 2: note = absent, L, K, 4-Pal, H or E
  SITE 3: note = absent, R, beta-Ala, P, K, E or G
  SITE 9: note = G, 4-Pal or H
  SITE 10: note = G, K, R or Dap
  SITE 11: note = R, K, G or Dap
  SITE 17: note = A, H, Dap, K, R or Orn
  SITE 18: note = Q, Y or 4-Pal
  SITE 24: note = need to insert options
  SITE 26: note = F or L
  SITE 28: note = Y, H or 4-Pal
  SITE 29: note = absent, GGP or selected from SEQ ID NO:4-20
  MOD_RES 29: note = C-terminal amino acid is optionally amidated
XXXRSSCFXXXIDRIGXXSGLGCXSXRXX

SEQ ID NO: 22 - protein, 7 aa, synthetic construct
EKEKEKG

SEQ ID NO: 23 - protein, 7 aa, synthetic construct
EPEPEPG

SEQ ID NO: 24 - protein, 5 aa, synthetic construct
APPSG

SEQ ID NO: 25 - protein, 6 aa, synthetic construct
KEKEKG

SEQ ID NO: 26 - protein, 7 aa, synthetic construct
EKEKEKE

SEQ ID NO: 27 - protein, 29 aa, synthetic construct
  SITE 1: note = S or E
  SITE 2: note = K or 4-Pal
  SITE 3: note = R, beta-Ala or K
  SITE 9: note = G
  SITE 10: note = G or K
  SITE 11: note = R or K
  SITE 13: note = D or G
  SITE 17: note = H
  SITE 18: note = Q or Y
  SITE 26: note = F
  SITE 28: note = H
  SITE 29: note = selected from SEQ ID NO:9, SEQ ID NO:11, and SEQ ID NO:13
  MOD_RES 29: note = C-terminal amino acid is optionally amidated
XXXRSSCFXXXIXRIGXXSGLGCPSXRXX

SEQ ID NO: 28 - protein, 36 aa, synthetic construct
  MOD_RES 1: note = N-terminal modified with HOOC-(CH2)18-CO-
  MOD_RES 36: note = AMIDATION
  DISULFID 15..31: note = Intrachain disulfide bond
  SITE 1: note = X at position 1 is gamma-Glu
XEKEKEKGSLRRSSCFGGRIDRIGAQSGLGCNSFRY

SEQ ID NO: 29 - protein, 36 aa, synthetic construct
  MOD_RES 1: note = N-terminal modified with HOOC-(CH2)18-CO-
  MOD_RES 36: note = AMIDATION
  DISULFID 15..31: note = Intrachain disulfide bond
  SITE 1: note = X at position 1 is gamma-Glu
  SITE 36: note = X at position 36 is 4-Pal
XEKEKEKGSLRRSSCFGGRIDRIGAQSGLGCNSFRX

SEQ ID NO: 30 - protein, 36 aa, synthetic construct
  SITE 1: note = X at position 1 is gamma-Glu
  SITE 26: note = X at position 26 is 4-Pal
  MOD_RES 36: note = Amidation
  MOD_RES 1: note = N-terminal modified with HOOC-(CH2)18-CO-
  DISULFID 15..31: note = Intrachain disulfide bond
XEKEKEKGSLRRSSCFGGRIDRIGAXSGLGCNSFRY

SEQ ID NO: 31 - protein, 36 aa, synthetic construct
  MOD_RES 1: note = N-terminal modified with HOOC-(CH2)18-CO-
  SITE 1: note = X at position 1 is gamma-Glu
  SITE 10: note = X at position 10 is 4-Pal
  MOD_RES 36: note = Amidation
  DISULFID 15..31: note = Intrachain disulfide bond
XEKEKEKGSXRRSSCFGGRIDRIGAQSGLGCNSFRY

SEQ ID NO: 32 - protein, 36 aa, synthetic construct
  MOD_RES 1: note = N-terminal modified with HOOC-(CH2)18-CO-
  SITE 1: note = X at position 1 is gamma-Glu
  SITE 26: note = X at position 26 is 4-Pal
  SITE 36: note = X at position 36 is 4-Pal
  MOD_RES 36: note = Amidation
  DISULFID 15..31: note = Intrachain disulfide bond
XEKEKEKGSLRRSSCFGGRIDRIGAXSGLGCNSFRX

SEQ ID NO: 33 - protein, 36 aa, synthetic construct
  SITE 1: note = X at position 1 is gamma-Glu
  MOD_RES 1: note = N-terminal modified with HOOC-(CH2)18-CO-
  SITE 17: note = X at position 17 is 4-Pal
  MOD_RES 36: note = Amidation
  DISULFID 15..31: note = Intrachain disulfide bond
XEKEKEKGSLRRSSCFXGRIDRIGAQSGLGCNSFRY

SEQ ID NO: 34 - protein, 36 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 26: note = X at position 26 is 4-Pal
  DISULFID 15..31: note = Intrachain disulfide bond
XEKEKEKGSXRRSSCFGGRIDRIGAXSGLGCNSFRY

SEQ ID NO: 35 - protein, 36 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 36: note = X at position 36 is 4-Pal
  MOD_RES 36: note = Amidation
  DISULFID 15..31: note = Intrachain disulfide bond
XEKEKEKGSXRRSSCFGGRIDRIGAQSGLGCNSFRX

SEQ ID NO: 36 - protein, 36 aa, synthetic construct
  SITE 1: note = X at position 1 is gamma-Glu
  MOD_RES 1: note = N-terminal modified with HOOC-(CH2)18-CO-
  MOD_RES 36: note = Amidation
  DISULFID 15..31: note = Intrachain disulfide bond
XEKEKEKGSHRRSSCFHGRIDRIHAQSGLGCNSFRH

SEQ ID NO: 37 - protein, 36 aa, synthetic construct
  SITE 1: note = X at position 1 is gamma-Glu
  MOD_RES 1: note = N-terminal modified with HOOC-(CH2)18-CO-
  MOD_RES 36: note = Amidation
  DISULFID 15..31: note = Intrachain thioacetal bond
XEKEKEKGSHRRSSCFGGRIDRIGAQSGLGCPSFRH

SEQ ID NO: 38 - protein, 36 aa, synthetic construct; identical to SEQ ID NO: 37
  SITE 1: note = X at position 1 is gamma-Glu
  MOD_RES 1: note = N-terminal modified with HOOC-(CH2)18-CO-
  MOD_RES 36: note = Amidation
  DISULFID 15..31: note = Intrachain disulfide bond

SEQ ID NO: 39 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGSXKRSSCFGGKIDRIGAQSGLGCPSFRHGGPSSGAPPPS

SEQ ID NO: 40 - protein, 36 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 36: note = X at position 36 is 4-Pal
  MOD_RES 36: note = Amidation
  DISULFID 15..31: note = Intrachain disulfide bond
XEKEKEKGSXRRSSCFGGRIDRIGAQSGLGCPSFRX

SEQ ID NO: 41 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGSXRRSSCFGGRIDRIGAQSGLGCPSFRHGGPSSGAPPPS

SEQ ID NO: 42 - protein, 36 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 36: note = Amidation
  DISULFID 15..31: note = Intrachain disulfide bond
XEKEKEKGSXRRSSCFGGRIDRIGAQSGLGCPSFRH

SEQ ID NO: 43 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 36: note = X at position 36 is 4-Pal
  MOD_RES 47: note = Amidation
  DISULFID 15..31: note = Intrachain disulfide bond
XEKEKEKGSXRRSSCFGGRIDRIGAQSGLGCPSFRXGGPSSGAPPPS

SEQ ID NO: 44 - protein, 36 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 36: note = X at position 36 is 4-Pal
  MOD_RES 36: note = Amidation
  DISULFID 15..31: note = Intrachain disulfide bond
XEKEKEKGSXRRSSCFGGRIDRIGHQSGLGCPSFRX

SEQ ID NO: 45 - protein, 47 aa, synthetic construct
  SITE 1: note = X at position 1 is gamma-Glu
  MOD_RES 1: note = N-terminal modified with HOOC-(CH2)18-CO-
  SITE 19: note = X at position 10 is 4-Pal
  MOD_RES 47: note = Amidation
  DISULFID 15..31: note = Intrachain disulfide bond
XEKEKEKGSXRRSSCFGGRIDRIGHQSGLGCPSFRHGGPSSGAPPPS

SEQ ID NO: 46 - protein, 48 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 48: note = Amidation
  DISULFID 15..31: note = Intrachain disulfide bond
XEKEKEKGSXRRSSCFGGRIDRIGHQSGLGCPSFRHRITAREDKQGYA

SEQ ID NO: 47 - protein, 48 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 48: note = Amidation
  DISULFID 15..31: note = Intrachain disulfide bond
XEKEKEKGSXRRSSCFGGRIDRIGHQSGLGCPSFRHRITAREDKQGEA

SEQ ID NO: 48 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 36: note = X at position 36 is 4-Pal
  MOD_RES 47: note = Amidation
  DISULFID 15..31: note = Intrachain disulfide bond
XEKEKEKGSXRRSSCFGGRIDRIGHQSGLGCPSFRXGGPSSGAPPPS

SEQ ID NO: 49 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGEXRRSSCFGGRIDRIGHQSGLGCPSFRHGGPSSGAPPPS

SEQ ID NO: 50 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGSXRRSSCFGGRIGRIGHQSGLGCPSFRHGGPSSGAPPPS

SEQ ID NO: 51 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGSXKRSSCFGGKIDRIGHQSGLGCPSFRHGGPSSGAPPPS

SEQ ID NO: 52 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 25: note = X at position 25 is Dap
  MOD_RES 47: note = Amidation
  DISULFID 15..31: note = Intrachain disulfide bond
XEKEKEKGSXKRSSCFGGKIDRIGXQSGLGCPSFRHGGPSSGAPPPS

SEQ ID NO: 53 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGEXKRSSCFGGKIDRIGHQSGLGCPSFRHGPSSGAPPPSE

SEQ ID NO: 54 - protein, 46 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 46: note = Amidation
  DISULFID 15..31: note = Intrachain disulfide bond
XEKEKEKGSXKRSSCFGGKIDRIGHQSGLGCPSFRHGPSSGAPPPS

SEQ ID NO: 55 - protein, 44 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 44: note = Amidation
  DISULFID 15..31: note = Intrachain disulfide bond
XEKEKEKGSXKRSSCFGGKIDRIGHQSGLGCPSFRHKITAKEDE

SEQ ID NO: 56 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGSXKRSSCFGGRIDRIGHQSGLGCPSFRHGGPSSGAPPPS

SEQ ID NO: 57 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGSXRRSSCFGGKIDRIGHQSGLGCPSFRHGGPSSGAPPPS

SEQ ID NO: 58 - protein, 46 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 46: note = Amidation
  DISULFID 15..31: note = Intrachain disulfide bond
XEKEKEKGSXRRSSCFGGRIDRIGHQSGLGCPSFRHGPSSGAPPPS

SEQ ID NO: 59 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain disulfide bond
XEKEKEKGSXRRSSCFGGRIDRIGHYSGLGCPSFRHGGPSSGAPPPS

SEQ ID NO: 60 - protein, 47 aa, synthetic construct; identical to SEQ ID NO: 45
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond

SEQ ID NO: 61 - protein, 44 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 44: note = Amidation
  DISULFID 15..31: note = Intrachain disulfide bond
XEKEKEKGSXRRSSCFGGRIDRIGHQSGLGCPSFRHSGAPPPSE

SEQ ID NO: 62 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain disulfide bond
XEKEKEKGEKERSSCFGGRIDRIGHQSGLGCPSFRHGGPSSGAPPPS

SEQ ID NO: 63 - protein, 44 aa, synthetic construct
  SITE 1: note = X at position 1 is gamma-Glu
  MOD_RES 1: note = N-terminal modified with HOOC-(CH2)18-CO-
  MOD_RES 44: note = Amidation
  DISULFID 12..28: note = Intrachain disulfide bond
XEKEKEKGRSSCFGGRIDRIGHQSGLGCPSFRHGGPSSGAPPPS

SEQ ID NO: 64 - protein, 46 aa, synthetic construct
  SITE 1: note = X at position 1 is gamma-Glu
  MOD_RES 1: note = N-terminal modified with HOOC-(CH2)18-CO-
  MOD_RES 46: note = Amidation
  DISULFID 15..31: note = Intrachain disulfide bond
XEKEKEKGEKGRSSCFGGRIDRIGHYSGLGCPSFRHGPSSGAPPPS

SEQ ID NO: 65 - protein, 44 aa, synthetic construct
  SITE 1: note = X at position 1 is gamma-Glu
  MOD_RES 1: note = N-terminal modified with HOOC-(CH2)18-CO-
  MOD_RES 44: note = Amidation
  DISULFID 12..28: note = Intrachain disulfide bond
XEKEKEKGRSSCFGGKIDRIGHYSGLGCPSFRHGGPSSGAPPPS

SEQ ID NO: 66 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain disulfide bond
XEKEKEKGEKERSSCFGGKIDRIGHYSGLGCPSFRHGGPSSGAPPPS

SEQ ID NO: 67 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain disulfide bond
XEKEKEKGEKGRSSCFGGKIDRIGHYSGLGCPSFRHGGPSSGAPPPS

SEQ ID NO: 68 - protein, 45 aa, synthetic construct
  SITE 1: note = X at position 1 is gamma-Glu
  MOD_RES 1: note = N-terminal modified with HOOC-(CH2)18-CO-
  MOD_RES 45: note = Amidation
  DISULFID 13..29: note = Intrachain disulfide bond
XEKEKEKGRRSSCFGGKIDRIGHYSGLGCPSFRHKGPSSGAPPPS

SEQ ID NO: 69 - protein, 44 aa, synthetic construct
  SITE 1: note = X at position 1 is gamma-Glu
  MOD_RES 1: note = N-terminal modified with HOOC-(CH2)18-CO-
  DISULFID 12..28: note = Intrachain disulfide bond
  MOD_RES 44: note = Amidation
XEKEKEKGRSSCFGGKIDRIGHYSGLGCPSFRHKGPSSGAPPPS

SEQ ID NO: 70 - protein, 46 aa, synthetic construct
  SITE 1: note = X at position 1 is gamma-Glu
  MOD_RES 1: note = N-terminal modified with HOOC-(CH2)18-CO-
  DISULFID 14..30: note = Intrachain disulfide bond
  MOD_RES 46: note = Amidation
XAPPPSGEKERSSCFGGKIDRIGHYSGLGCPSFRHGGPSSGAPPPS

SEQ ID NO: 71 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain disulfide bond
XEKEKEKGEKERSSCFGGRIDRIGHYSGLGCPSFRHGGPSSGAPPPS

SEQ ID NO: 72 - protein, 48 aa, synthetic construct
  SITE 1: note = X at position 1 is gamma-Glu
  MOD_RES 1: note = N-terminal modified with HOOC-(CH2)18-CO-
  MOD_RES 48: note = Amidation
  DISULFID 15..31: note = Intrachain disulfide bond
XEKEKEKGEKGRSSCFGGRIDRIGHYSGLGCPSFRHKGGPSSGAPPPS

SEQ ID NO: 73 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain disulfide bond
XEKEKEKGEKGRSSCFGGRIDRIGHYSGLGCPSLRHGGPSSGAPPPS

SEQ ID NO: 74 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain disulfide bond
XEKEKEKGEKGRSSCFGGKIDRIGKYSGLGCPSFRHGGPSSGAPPPS

SEQ ID NO: 75 - protein, 46 aa, synthetic construct
  SITE 1: note = X at position 1 is gamma-Glu
  MOD_RES 1: note = N-terminal modified with HOOC-(CH2)18-CO-
  MOD_RES 46: note = Amidation
  DISULFID 15..31: note = Intrachain disulfide bond
XEKEKEKGEKGRSSCFGGRIDRIGKYSGLGCPSFRHGPSSGAPPPS

SEQ ID NO: 76 - protein, 47 aa, synthetic construct
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain disulfide bond
XEKEKEKGEKXRSSCFGGRIDRIGHYSGLGCPSFRHGGPSSGAPPPS

SEQ ID NO: 77 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain disulfide bond
XEKEKEKGEKPRSSCFGGRIDRIGHYSGLGCPSFRHGGPSSGAPPPS

SEQ ID NO: 78 - protein, 47 aa, synthetic construct; identical to SEQ ID NO: 76
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond

SEQ ID NO: 79 - protein, 47 aa, synthetic construct; identical to SEQ ID NO: 77
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain thioacetal bond

SEQ ID NO: 80 - protein, 47 aa, synthetic construct
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond
XEKEKEKGKEXRSSCFGGRIDRIGHYSGLGCPSFRHGGPSSGAPPPS

SEQ ID NO: 81 - protein, 47 aa, synthetic construct
  SITE 1: note = X at position 1 is gamma-Glu
  MOD_RES 47: note = Amidation
  DISULFID 15..31: note = Intrachain thioacetal bond
  MOD_RES 1: note = N-terminal modified with HOOC-(CH2)18-CO-
XEKEKEKGEKGRSSCFGKRIDRIGHYSGLGCPSFRHGGPSSGAPPPS

SEQ ID NO: 82 - protein, 46 aa, synthetic construct
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 46: note = Amidation
  DISULFID 15..31: note = Intrachain thioacetal bond
XEKEKEKGEKXRSSCFGGRIDRIGHYSGLGCPSFRHGPSSGAPPPS

SEQ ID NO: 83 - protein, 39 aa, synthetic construct
  MOD_RES 1: note = N-terminal modified with HOOC-(CH2)18-CO-(gamma-Glu)-EKEKEKG-PEG24-
  SITE 2: note = X at position 2 is 4-Pal
  MOD_RES 39: note = Amidation
  DISULFID 7..23: note = Intrachain thioacetal bond
SXRRSSCFGGRIDRIGHQSGLGCPSFRHGGPSSGAPPPS

SEQ ID NO: 84 - protein, 39 aa, synthetic construct
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond
EKXRSSCFGGRIDRIGHYSGLGCPSFRHGGPSSGAPPPS

SEQ ID NO: 85 - protein, 47 aa, synthetic construct
  SITE 1: note = X at position 1 is gamma-Glu
  SITE 11: note = X at position 11 is beta-Alanine
  MOD_RES 47: note = Amidation
  DISULFID 15..31: note = Intrachain thioacetal bond
  MOD_RES 1: note = N-terminal modified with HOOC-(CH2)18-CO-
XEKEKEKGEKXRSSCFGGRIDRIGHYSGLGCPSLRHGGPSSGAPPPS

SEQ ID NO: 86 - protein, 47 aa, synthetic construct
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond
XEKEKEKGSKXRSSCFGKRIDRIGHYSGLGCPSFRHGGPSSGAPPPS

SEQ ID NO: 87 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain thioacetal bond
XEKEKEKGSKXRSSCFGGRIDRIGHYSGLGCPSFRHGGPSSGAPPPS

SEQ ID NO: 88 - protein, 46 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 46: note = Amidation
  DISULFID 15..31: note = Intrachain thioacetal bond
XEKEKEKGSXRRSSCFGGRIDRIGHQSGLGCPSFRHGGSSGAPPPS

SEQ ID NO: 89 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond
XEKEKEKGSXRRSSCFGGRIDRIGHQSGLGCPSFRHGGKSSGAPPPS

SEQ ID NO: 90 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  SITE 42: note = X at position 42 is Aib
  MOD_RES 47: note = Amidation
  DISULFID 15..31: note = Intrachain thioacetal bond
XEKEKEKGSXRRSSCFGGRIDRIGHQSGLGCPSFRHGGPPSXKPPPK

SEQ ID NO: 91 - protein, 47 aa, synthetic construct; identical to SEQ ID NO: 45
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond

SEQ ID NO: 92 - protein, 47 aa, synthetic construct; identical to SEQ ID NO: 76
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond

SEQ ID NO: 93 - protein, 47 aa, synthetic construct; identical to SEQ ID NO: 90
  SITE 1: note = X at position 1 is gamma-Glu
  MOD_RES 1: note = N-terminal modified with HOOC-(CH2)18-CO-
  SITE 10: note = X at position 10 is 4-Pal
  SITE 42: note = X at position 42 is Aib
  MOD_RES 47: note = Amidation
  DISULFID 15..31: note = Intrachain thioacetal bond

SEQ ID NO: 94 - protein, 47 aa, synthetic construct; identical to SEQ ID NO: 89
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond

SEQ ID NO: 95 - protein, 47 aa, synthetic construct
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond
XEKEKEKGEKXRSSCFGGRIDRIGHYSGLGCPSFRHGGKSSGAPPPS

SEQ ID NO: 96 - protein, 47 aa, synthetic construct; identical to SEQ ID NO: 95
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond

SEQ ID NO: 97 - protein, 39 aa, synthetic construct
  MOD_RES 1: note = N-terminal modified with HOOC-(CH2)18-CO-(gamma-Glu)-EKEKEKG-PEG24-
  SITE 2: note = X at position 2 is 4-Pal
  MOD_RES 39: note = Amidation
  DISULFID 7..23: note = Intrachain thioacetal bond
SXRRSSCFGGRIDRIGHQSGLGCPSFRHGGKSSGAPPPS

SEQ ID NO: 98 - protein, 47 aa, synthetic construct; identical to SEQ ID NO: 95
  SITE 1: note = X at position 1 is gamma-Glu
  MOD_RES 1: note = N-terminal modified with HOOC-(CH2)18-CO-
  SITE 11: note = X at position 11 is beta-Alanine
  DISULFID 15..31: note = Intrachain thioacetal bond

SEQ ID NO: 99 - protein, 39 aa, synthetic construct
  MOD_RES 1: note = N-terminal modified with HOOC-(CH2)18-CO-(gamma-Glu)-EKEKEKG-PEG12-
  SITE 3: note = X at position 3 is beta-Alanine
  MOD_RES 39: note = Amidation
  DISULFID 7..23: note = Intrachain thioacetal bond
EKXRSSCFGGRIDRIGHYSGLGCPSFRHGGKSSGAPPPS

SEQ ID NO: 100 - protein, 39 aa, synthetic construct; identical to SEQ ID NO: 99
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond

SEQ ID NO: 101 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond
XEKEKEKGSXRRSSCFGKRIDRIGHQSGLGCPSFRHGGKSSGAPPPS

SEQ ID NO: 102 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond
XEKEKEKGSXRRSSCFGRKIDRIGHQSGLGCPSFRHGGKSSGAPPPS

SEQ ID NO: 103 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond
XEKEKEKGSXRRSSCFGKRIDRIGHYSGLGCPSFRHGGKSSGAPPPS

SEQ ID NO: 104 - protein, 39 aa, synthetic construct; identical to SEQ ID NO: 97
  MOD_RES 1: note = N-terminal modified with HOOC-(CH2)18-CO-(gamma-Glu)-EKEKEKG-PEG12-
  SITE 2: note = X at position 2 is 4-Pal
  MOD_RES 39: note = Amidation
  DISULFID 7..23: note = Intrachain thioacetal bond

SEQ ID NO: 105 - protein, 39 aa, synthetic construct; identical to SEQ ID NO: 97
  MOD_RES 1: note = N-terminal modified with HOOC-(CH2)18-CO-(gamma-Glu)-EKEKEKG-(AEEA)4-
  SITE 2: note = X at position 2 is 4-Pal
  MOD_RES 39: note = Amidation
  DISULFID 7..23: note = Intrachain thioacetal bond

SEQ ID NO: 106 - protein, 39 aa, synthetic construct
  MOD_RES 1: note = N-terminal modified with HOOC-(CH2)18-CO-(gamma-Glu)-EKEKEKG-PEG24-
  SITE 2: note = X at position 2 is 4-Pal
  SITE 34: note = X at position 34 is Aib
  MOD_RES 39: note = Amidation
  DISULFID 7..23: note = Intrachain thioacetal bond
SXRRSSCFGGRIDRIGHQSGLGCPSFRHGGPPSXKPPPK

SEQ ID NO: 107 - protein, 39 aa, synthetic construct
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond
EKXRSSCFGKRIDRIGHQSGLGCPSFRHGGKSSGAPPPS

SEQ ID NO: 108 - protein, 47 aa, synthetic construct
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond
XEKEKEKGEKXRSSCFGKRIDRIGHQSGLGCPSFRHGGKSSGAPPPS

SEQ ID NO: 109 - protein, 39 aa, synthetic construct; identical to SEQ ID NO: 97
  MOD_RES 1: note = N-terminal modified with HOOC-(CH2)18-CO-(gamma-Glu)-EKEKEKG-(AEEA)6-
  SITE 2: note = X at position 2 is 4-Pal
  MOD_RES 39: note = Amidation
  DISULFID 7..23: note = Intrachain thioacetal bond

SEQ ID NO: 110 - protein, 39 aa, synthetic construct
  MOD_RES 1: note = N-terminal modified with HOOC-(CH2)18-CO-(gamma-Glu)-EKEKEKG-(AEEA)8-
  SITE 2: note = X at position 2 is 4-Pal
  MOD_RES 39: note = Amidation
  DISULFID 7..23: note = Intrachain thioacetal bond
SXRRSSCFGKRIDRIGHQSGLGCPSFRHGGKSSGAPPPS

SEQ ID NO: 111 - protein, 39 aa, synthetic construct; identical to SEQ ID NO: 107
  MOD_RES 1: note = N-terminal modified with HOOC-(CH2)18-CO-(gamma-Glu)-EKEKEKG-(AEEA)4-
  SITE 3: note = X at position 3 is beta-Alanine
  MOD_RES 39: note = Amidation
  DISULFID 7..23: note = Intrachain thioacetal bond

SEQ ID NO: 112 - protein, 39 aa, synthetic construct; identical to SEQ ID NO: 107
  MOD_RES 1: note = N-terminal modified with HOOC-(CH2)18-CO-(gamma-Glu)-EKEKEKG-(AEEA)6-
  SITE 3: note = X at position 3 is beta-Alanine
  MOD_RES 39: note = Amidation
  DISULFID 7..23: note = Intrachain thioacetal bond

SEQ ID NO: 113 - protein, 39 aa, synthetic construct; identical to SEQ ID NO: 107
  MOD_RES 1: note = N-terminal modified with HOOC-(CH2)18-CO-(gamma-Glu)-EKEKEKG-(AEEA)8-
  SITE 3: note = X at position 3 is beta-Alanine
  MOD_RES 39: note = Amidation
  DISULFID 7..23: note = Intrachain thioacetal bond

SEQ ID NO: 114 - protein, 39 aa, synthetic construct
  MOD_RES 1: note = N-terminal modified with HOOC-(CH2)18-CO-(gamma-Glu)-EKEKEKG-(AEEA)8-
  SITE 2: note = X at position 2 is 4-Pal
  MOD_RES 39: note = Amidation
  DISULFID 7..23: note = Intrachain thioacetal bond
SXRRSSCFGGRIDRIGHYSGLGCPSFRHGGKSSGAPPPS

SEQ ID NO: 115 - protein, 39 aa, synthetic construct; identical to SEQ ID NO: 114
  MOD_RES 1: note = N-terminal modified with HOOC-(CH2)18-CO-(gamma-Glu)-EKEKEKG-(AEEA)6-
  SITE 2: note = X at position 2 is 4-Pal
  MOD_RES 39: note = Amidation
  DISULFID 7..23: note = Intrachain thioacetal bond

SEQ ID NO: 116 - protein, 39 aa, synthetic construct; identical to SEQ ID NO: 99
  MOD_RES 1: note = N-terminal modified with HOOC-(CH2)18-CO-(gamma-Glu)-EKEKEKG-(AEEA)8-
  MOD_RES 39: note = Amidation
  SITE 3: note = X at position 3 is beta-Alanine
  DISULFID 7..23: note = Intrachain thioacetal bond

SEQ ID NO: 117 - protein, 39 aa, synthetic construct; identical to SEQ ID NO: 99
  MOD_RES 1: note = N-terminal modified with HOOC-(CH2)18-CO-(gamma-Glu)-EKEKEKG-(AEEA)6-
  SITE 3: note = X at position 3 is beta-Alanine
  MOD_RES 39: note = Amidation
  DISULFID 7..23: note = Intrachain thioacetal bond

SEQ ID NO: 118 - protein, 39 aa, synthetic construct; identical to SEQ ID NO: 114
  MOD_RES 1: note = N-terminal modified with HOOC-(CH2)18-CO-(gamma-Glu)-EKEKEKG-PEG24-
  SITE 2: note = X at position 2 is 4-Pal
  MOD_RES 39: note = Amidation
  DISULFID 7..23: note = Intrachain thioacetal bond

SEQ ID NO: 119 - protein, 39 aa, synthetic construct
  MOD_RES 1: note = N-terminal modified with HOOC-(CH2)18-CO-(gamma-Glu)-EKEKEKG-(AEEA)6-
  SITE 2: note = X at position 2 is 4-Pal
  MOD_RES 39: note = Amidation
  DISULFID 7..23: note = Intrachain thioacetal bond
SXRRSSCFGGRIDRIGHYSGLGCPSFRHGSPSSGAPPPS

SEQ ID NO: 120 - protein, 39 aa, synthetic construct
  MOD_RES 1: note = N-terminal modified with HOOC-(CH2)18-CO-(gamma-Glu)-EKEKEKG-(AEEA)6-
  SITE 2: note = X at position 2 is 4-Pal
  SITE 34: note = X at position 34 is Aib
  MOD_RES 39: note = Amidation
  DISULFID 7..23: note = Intrachain thioacetal bond
SXRRSSCFGGRIDRIGHYSGLGCPSFRHGGPPSXKPPPK

SEQ ID NO: 121 - protein, 39 aa, synthetic construct
  MOD_RES 1: note = N-terminal modified with HOOC-(CH2)18-CO-(gamma-Glu)-EKEKEKG-(AEEA)6-
  SITE 3: note = X at position 3 is beta-Alanine
  MOD_RES 39: note = Amidation
  DISULFID 7..23: note = Intrachain thioacetal bond
EKXRSSCFGGRIDRIGHYSGLGCPSFRHGSPSSGAPPPS

SEQ ID NO: 122 - protein, 39 aa, synthetic construct
  MOD_RES 1: note = N-terminal modified with HOOC-(CH2)18-CO-(gamma-Glu)-EKEKEKG-(AEEA)4-
  SITE 3: note = X at position 3 is beta-Alanine
  MOD_RES 39: note = Amidation
  DISULFID 7..23: note = Intrachain thioacetal bond
EKXRSSCFGKRIDRIGHYSGLGCPSFRHGGKSSGAPPPS

SEQ ID NO: 123 - protein, 39 aa, synthetic construct; identical to SEQ ID NO: 122
  MOD_RES 1: note = N-terminal modified with HOOC-(CH2)18-CO-(gamma-Glu)-EKEKEKG-(AEEA)6-
  SITE 3: note = X at position 3 is beta-Alanine
  MOD_RES 39: note = Amidation
  DISULFID 7..23: note = Intrachain thioacetal bond

SEQ ID NO: 124 - protein, 47 aa, synthetic construct
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond
XEKEKEKGEKXRSSCFGKRIDRIGHYSGLGCPSFRHGSPSSGAPPPS

SEQ ID NO: 125 - protein, 39 aa, synthetic construct; identical to SEQ ID NO: 122
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond

SEQ ID NO: 126 - protein, 39 aa, synthetic construct
  MOD_RES 1: note = N-terminal modified with HOOC-(CH2)18-CO-(gamma-Glu)-EKEKEKG-(AEEA)4-
  SITE 3: note = X at position 3 is beta-Alanine
  MOD_RES 39: note = Amidation
  DISULFID 7..23: note = Intrachain thioacetal bond
EKXRSSCFGKRIDRIGHYSGLGCPSFRHGSPSSGAPPPS

SEQ ID NO: 127 - protein, 39 aa, synthetic construct
  MOD_RES 1: note = N-terminal modified with HOOC-(CH2)18-CO-(gamma-Glu)-EKEKEKG-(AEEA)6-
  SITE 2: note = X at position 2 is 4-Pal
  MOD_RES 39: note = Amidation
  DISULFID 7..23: note = Intrachain thioacetal bond
SXRRSSCFGKRIDRIGHYSGLGCPSFRHGGKSSGAPPPS

SEQ ID NO: 128 - protein, 39 aa, synthetic construct; identical to SEQ ID NO: 126
  MOD_RES 1: note = N-terminal modified with HOOC-(CH2)18-CO-(gamma-Glu)-EKEKEKG-(AEEA)6-
  SITE 3: note = X at position 3 is beta-Alanine
  MOD_RES 39: note = Amidation
  DISULFID 7..23: note = Intrachain thioacetal bond

SEQ ID NO: 129 - protein, 39 aa, synthetic construct; identical to SEQ ID NO: 126
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond

SEQ ID NO: 130 - protein, 39 aa, synthetic construct
  MOD_RES 1: note = N-terminal modified with HOOC-(CH2)18-CO-(gamma-Glu)-EKEKEKG-(AEEA)8-
  SITE 2: note = X at position 2 is 4-Pal
  MOD_RES 39: note = Amidation
  DISULFID 7..23: note = Intrachain thioacetal bond
SXRRSSCFGKRIDRIGHYSGLGCPSFRHGSPSSGAPPPS

SEQ ID NO: 131 - protein, 39 aa, synthetic construct; identical to SEQ ID NO: 126
  MOD_RES 1: note = N-terminal modified with HOOC-(CH2)18-CO-(gamma-Glu)-EKEKEKG-(AEEA)8-
  SITE 3: note = X at position 3 is beta-Alanine
  MOD_RES 39: note = Amidation
  DISULFID 7..23: note = Intrachain thioacetal bond

SEQ ID NO: 132 - protein, 39 aa, synthetic construct; identical to SEQ ID NO: 110
  MOD_RES 1: note = N-terminal modified with HOOC-(CH2)18-CO-(gamma-Glu)-EKEKEKG-PEG24-
  SITE 2: note = X at position 2 is 4-Pal
  MOD_RES 39: note = Amidation
  DISULFID 7..23: note = Intrachain thioacetal bond

SEQ ID NO: 133 - protein, 39 aa, synthetic construct; identical to SEQ ID NO: 127
  MOD_RES 1: note = N-terminal modified with HOOC-(CH2)18-CO-(gamma-Glu)-EKEKEKG-PEG24-
  SITE 2: note = X at position 2 is 4-Pal
  MOD_RES 39: note = Amidation
  DISULFID 7..23: note = Intrachain thioacetal bond

SEQ ID NO: 134 - protein, 39 aa, synthetic construct
  MOD_RES 1: note = N-terminal modified with HOOC-(CH2)18-CO-(gamma-Glu)-EKEKEKG-(AEEA)6-
  SITE 2: note = X at position 2 is 4-Pal
  SITE 3: note = X at position 3 is beta-Alanine
  MOD_RES 39: note = Amidation
  DISULFID 7..23: note = Intrachain thioacetal bond
SXXRSSCFGGRIDRIGHYSGLGCPSFRHGSPSSGAPPPS

SEQ ID NO: 135 - protein, 47 aa, synthetic construct
  SITE 1: note = X at position 1 is gamma-Glu
  MOD_RES 1: note = N-terminal modified with HOOC-(CH2)18-CO-
  SITE 10: note = X at position 10 is 4-Pal
  MOD_RES 47: note = Amidation
  DISULFID 15..31: note = Intrachain thioacetal bond
XEKEKEKGSXRRSSCFGKRIDRIGHYSGLGCPSFRHGSPSSGAPPPS

SEQ ID NO: 136 - protein, 39 aa, synthetic construct; identical to SEQ ID NO: 130
  MOD_RES 1: note = N-terminal modified with HOOC-(CH2)18-CO-(gamma-Glu)-EKEKEKG-(AEEA)6-
  SITE 2: note = X at position 2 is 4-Pal
  MOD_RES 39: note = Amidation
  DISULFID 7..23: note = Intrachain thioacetal bond

SEQ ID NO: 137 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain thioacetal bond
XEKEKEKGEKPRSSCFGKRIDRIGHYSGLGCPSFRHGSPSSGAPPPS

SEQ ID NO: 138 - protein, 39 aa, synthetic construct; identical to SEQ ID NO: 130
  MOD_RES 1: note = N-terminal modified with HOOC-(CH2)18-CO-(gamma-Glu)-EKEKEKG-PEG24-
  SITE 2: note = X at position 2 is 4-Pal
  MOD_RES 39: note = Amidation
  DISULFID 7..23: note = Intrachain thioacetal bond

SEQ ID NO: 139 - protein, 41 aa, synthetic construct
  MOD_RES 1: note = N-terminal modified with HOOC-(CH2)18-CO-(gamma-Glu)-EKEKEKG-(AEEA)6-
  MOD_RES 41: note = Amidation
  DISULFID 7..23: note = Intrachain thioacetal bond
EKPRSSCFGKRIDRIGHYSGLGCPSFRHSGSPSSGAPPPSG

SEQ ID NO: 140 - protein, 42 aa, synthetic construct
  MOD_RES 1: note = N-terminal modified with HOOC-(CH2)18-CO-(gamma-Glu)-EKEKEKG-(AEEA)6-
  SITE 2: note = X at position 2 is 4-Pal
  MOD_RES 42: note = Amidation
  DISULFID 7..23: note = Intrachain thioacetal bond
SXRRSSCFGKRIDRIGHYSGLGCPSFRHGSGSPSSGAPPPSG

SEQ ID NO: 141 - protein, 39 aa, synthetic construct; identical to SEQ ID NO: 121
  MOD_RES 1: note = N-terminal modified with HOOC-(CH2)18-CO-(gamma-Glu)-EKEKEKG-(AEEA)8-
  SITE 3: note = X at position 3 is beta-Alanine
  MOD_RES 39: note = Amidation
  DISULFID 7..23: note = Intrachain thioacetal bond

SEQ ID NO: 142 - protein, 39 aa, synthetic construct
  MOD_RES 1: note = N-terminal modified with HOOC-(CH2)18-CO-(gamma-Glu)-EKEKEKG-(AEEA)6-
  SITE 3: note = X at position 3 is beta-Alanine
  MOD_RES 39: note = Amidation
  DISULFID 7..23: note = Intrachain thioacetal bond
EKXRSSCFGKRIDRIGHQSGLGCPSFRHGSPSSGAPPPS

SEQ ID NO: 143 - protein, 39 aa, synthetic construct; identical to SEQ ID NO: 142
  MOD_RES 1: note = N-terminal modified with HOOC-(CH2)18-CO-(gamma-Glu)-EKEKEKG-(AEEA)8-
  SITE 3: note = X at position 3 is beta-Alanine
  MOD_RES 39: note = Amidation
  DISULFID 7..23: note = Intrachain thioacetal bond

SEQ ID NO: 144 - protein, 39 aa, synthetic construct; identical to SEQ ID NO: 142
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond

SEQ ID NO: 145 - protein, 39 aa, synthetic construct; identical to SEQ ID NO: 121
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond

SEQ ID NO: 146 - protein, 47 aa, synthetic construct; identical to SEQ ID NO: 137
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain thioacetal bond

SEQ ID NO: 147 - protein, 39 aa, synthetic construct
  MOD_RES 1: note = N-terminal modified with HOOC-(CH2)18-CO-(gamma-Glu)-EKEKEKG-(AEEA)6-
  MOD_RES 39: note = Amidation
  DISULFID 7..23: note = Intrachain thioacetal bond
EKPRSSCFGKRIDRIGHYSGLGCPSFRHGSPSSGAPPPS

SEQ ID NO: 148 - protein, 39 aa, synthetic construct; identical to SEQ ID NO: 147
  MOD_RES 1: note = N-terminal modified with HOOC-(CH2)18-CO-(gamma-Glu)-EKEKEKG-(AEEA)8-
  MOD_RES 39: note = Amidation
  DISULFID 7..23: note = Intrachain thioacetal bond

SEQ ID NO: 149 - protein, 41 aa, synthetic construct; identical to SEQ ID NO: 139
  MOD_RES 1: note = N-terminal modified with HOOC-(CH2)18-CO-(gamma-Glu)-EKEKEKG-PEG24-
  MOD_RES 41: note = Amidation
  DISULFID 7..23: note = Intrachain thioacetal bond

SEQ ID NO: 150 - protein, 39 aa, synthetic construct; identical to SEQ ID NO: 147
  MOD_RES 1: note = N-terminal modified with HOOC-(CH2)18-CO-(gamma-Glu)-EKEKEKG-PEG24-
  MOD_RES 39: note = Amidation
  DISULFID 7..23: note = Intrachain thioacetal bond

SEQ ID NO: 151 - protein, 41 aa, synthetic construct; identical to SEQ ID NO: 139
  MOD_RES 1: note = N-terminal modified with HOOC-(CH2)18-CO-(gamma-Glu)-EKEKEKG-(AEEA)8-
  MOD_RES 41: note = Amidation
  DISULFID 7..23: note = Intrachain thioacetal bond

SEQ ID NO: 152 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain thioacetal bond
XEKEKEKGEKPRSSCFGKRIDRIGHQSGLGCPSFRHGSPSSGAPPPS

SEQ ID NO: 153 - protein, 47 aa, synthetic construct; identical to SEQ ID NO: 124
  SITE 1: note = X at position 1 is gamma-Glu
  MOD_RES 1: note = N-terminal modified with HOOC-(CH2)18-CO-
  SITE 11: note = X at position 11 is beta-Alanine
  MOD_RES 47: note = Amidation
  DISULFID 15..31: note = Intrachain thioacetal bond

SEQ ID NO: 154 - protein, 31 aa, synthetic construct
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 31: note = Amidation
  DISULFID 7..23: note = Intrachain thioacetal bond
EKXRSSCFGKRIDRIGHYSGLGCPSFRHGGP

SEQ ID NO: 155 - protein, 31 aa, synthetic construct
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 31: note = Amidation
  DISULFID 7..23: note = Intrachain thioacetal bond
EKXRSSCFGKRIDRIGHQSGLGCPSFRHGGP

SEQ ID NO: 156 - protein, 50 aa, synthetic construct
  SITE 1: note = X at position 1 is gamma-Glu
  MOD_RES 1: note = N-terminal modified with HOOC-(CH2)18-CO-
  MOD_RES 50: note = Amidation
  DISULFID 15..31: note = Intrachain thioacetal bond
XEKEKEKGEKPRSSCFGKRIDRIGHQSGLGCPSFRHGSGSPSSGAPPPSG

SEQ ID NO: 157 - protein, 41 aa, synthetic construct
  MOD_RES 1: note = N-terminal modified with HOOC-(CH2)18-CO-(gamma-Glu)-EKEKEKG-PEG24-
  MOD_RES 41: note = Amidation
  DISULFID 7..23: note = Intrachain thioacetal bond
EKPRSSCFGKRIDRIGHQSGLGCPSFRHSGSPSSGAPPPSG

SEQ ID NO: 158 - protein, 39 aa, synthetic construct
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond
EKXRSSCFGGKIDRIGHYSGLGCPSFRHGSPSSGAPPPS

SEQ ID NO: 159 - protein, 39 aa, synthetic construct
  MOD_RES 1: note = N-terminal modified with HOOC-(CH2)18-CO-(gamma-Glu)-EKEKEKG-PEG24-
  SITE 3: note = X at position 3 is beta-Alanine
  MOD_RES 39: note = Amidation
  DISULFID 7..23: note = Intrachain thioacetal bond
EKXRSSCFGGKIDRIGHQSGLGCPSFRHGSPSSGAPPPS

SEQ ID NO: 160 - protein, 41 aa, synthetic construct
  MOD_RES 1: note = N-terminal modified with HOOC-(CH2)18-CO-(gamma-Glu)-EKEKEKG-PEG24-
  MOD_RES 41: note = Amidation
  DISULFID 7..23: note = Intrachain thioacetal bond
EKPRSSCFGKGIDRIGHQSGLGCPSFRHSGSPSSGAPPPSG

SEQ ID NO: 161 - protein, 47 aa, synthetic construct
  SITE 1: note = X at position 1 is gamma-Glu
  MOD_RES 1: note = N-terminal modified with HOOC-(CH2)18-CO-
  SITE 25: note = X at position 25 is Orn
  MOD_RES 47: note = Amidation
  DISULFID 15..31: note = Intrachain thioacetal bond
XEKEKEKGEKPRSSCFGKRIDRIGXQSGLGCPSFRHGSPSSGAPPPS

SEQ ID NO: 162 - protein, 47 aa, synthetic construct
  SITE 1: note = X at position 1 is gamma-Glu
  MOD_RES 1: note = N-terminal modified with HOOC-(CH2)18-CO-
  SITE 25: note = X at position 25 is Orn
  MOD_RES 47: note = Amidation
  DISULFID 15..31: note = Intrachain thioacetal bond
XEKEKEKGEKPRSSCFGKRIDRIGXYSGLGCPSFRHGSPSSGAPPPS

SEQ ID NO: 163 - protein, 47 aa, synthetic construct
  SITE 1: note = X at position 1 is gamma-Glu
  MOD_RES 1: note = N-terminal modified with HOOC-(CH2)18-CO-
  SITE 18: note = X at position 18 is Dap
  MOD_RES 47: note = Amidation
  DISULFID 15..31: note = Intrachain thioacetal bond
XEKEKEKGEKPRSSCFGXRIDRIGHYSGLGCPSFRHGSPSSGAPPPS

SEQ ID NO: 164 - protein, 47 aa, synthetic construct
  SITE 1: note = X at position 1 is gamma-Glu
  MOD_RES 1: note = N-terminal modified with HOOC-(CH2)18-CO-
  SITE 18: note = X at position 18 is Dap
  MOD_RES 47: note = Amidation
  DISULFID 15..31: note = Intrachain thioacetal bond
XEKEKEKGEKPRSSCFGXRIDRIGHQSGLGCPSFRHGSPSSGAPPPS

SEQ ID NO: 165 - protein, 47 aa, synthetic construct
  SITE 1: note = X at position 1 is gamma-Glu
  MOD_RES 1: note = N-terminal modified with HOOC-(CH2)18-CO-
  SITE 19: note = X at position 19 is Dap
  MOD_RES 47: note = Amidation
  DISULFID 15..31: note = Intrachain thioacetal bond
XEKEKEKGEKPRSSCFGGXIDRIGHYSGLGCPSFRHGSPSSGAPPPS

SEQ ID NO: 166 - protein, 47 aa, synthetic construct
  SITE 1: note = X at position 1 is gamma-Glu
  MOD_RES 1: note = N-terminal modified with HOOC-(CH2)18-CO-
  SITE 19: note = X at position 19 is Dap
  MOD_RES 47: note = Amidation
  DISULFID 15..31: note = Intrachain thioacetal bond
XEKEKEKGEKPRSSCFGGXIDRIGHQSGLGCPSFRHGSPSSGAPPPS

SEQ ID NO: 167 - protein, 47 aa, synthetic construct
  SITE 1: note = X at position 1 is gamma-Glu
  MOD_RES 1: note = N-terminal modified with HOOC-(CH2)18-CO-
  MOD_RES 47: note = Amidation
  DISULFID 15..31: note = Intrachain thioacetal bond
XEKEKEKGEKPRSSCFGKRIDRIGRYSGLGCPSFRHGSPSSGAPPPS

SEQ ID NO: 168 - protein, 46 aa, synthetic construct
  MOD_RES 1: note = N-terminal modified with Bifurcated Fatty Acid (BFA)
  MOD_RES 46: note = Amidation
  DISULFID 14..30: note = Intrachain disulfide bond
EKEKEKGEKGRSSCFGGKIDRIGHYSGLGCPSFRHGGPSSGAPPPS

SEQ ID NO: 169 - protein, 39 aa, synthetic construct
  MOD_RES 1: note = N-terminal modified with (BFAEN2-beta-Ala)-EKEKEKG-PEG24-; wherein BFAEN2 is Bifurcated Fatty Acid Enantiomer 2
  MOD_RES 39: note = Amidation
  DISULFID 7..23: note = Intrachain thioacetal bond
  SITE 3: note = X at position 3 is beta-Alanine
EKXRSSCFGGKIDRIGHYSGLGCPSFRHGGKSSGAPPPS

SEQ ID NO: 170 - protein, 39 aa, synthetic construct
  MOD_RES 1: note = N-terminal modified with (BFAEN2-beta-Ala)-EKEKEKG-PEG24-; wherein BFAEN2 is Bifurcated Fatty Acid Enantiomer 2
  SITE 2: note = X at position 2 is 4-Pal
  MOD_RES 39: note = Amidation
EXKRSSCFGKKIDRIGHYSGLGCPSFRHGGKSSGAPPPS

SEQ ID NO: 171 - protein, 39 aa, synthetic construct; identical to SEQ ID NO: 142
  MOD_RES 1: note = N-terminal modified with (BFAEN2-beta-Ala)-EKEKEKG-PEG24-; wherein BFAEN2 is Bifurcated Fatty Acid Enantiomer 2
  SITE 3: note = X at position 3 is beta-Alanine
  MOD_RES 39: note = Amidation
  DISULFID 7..23: note = Intrachain thioacetal bond

SEQ ID NO: 172 - protein, 39 aa, synthetic construct; identical to SEQ ID NO: 158
  MOD_RES 1: note = N-terminal modified with (BFAEN2-beta-Ala)-KEKEKG-PEG24-; wherein BFAEN2 is Bifurcated Fatty Acid Enantiomer 2
  SITE 3: note = X at position 3 is beta-Alanine
  MOD_RES 39: note = Amidation
  DISULFID 7..23: note = Intrachain thioacetal bond

SEQ ID NO: 173 - protein, 28 aa, Rattus norvegicus
  MOD_RES 28: note = Amidation
  DISULFID 7..23: note = Intrachain disulfide bond
SLRRSSCFGGRIDRIGAQSGLGCNSFRY

(19) United States
(12) Patent Application Publication
(10) Pub. No.: US 2024/0174727 A1
(43) Pub. Date: May 30, 2024

(54) LONG-ACTING NATRIURETIC PEPTIDES AND USES THEREOF

(71) Applicant: Eli Lilly and Company, Indianapolis, IN (US)

(72) Inventors: Jorge ALSINA-FERNANDEZ, Indianapolis, IN (US); Hana Elisabeth BAKER, Westfield, IN (US); Guillermo S. CORTEZ, Indianapolis, IN (US); Michael Lawrence ELMUCCIO, Carmel, IN (US); Wen LIU, Carmel, IN (US); Daniel Christopher LOPES, Indianapolis, IN (US); Avinash MUPPIDI, Carmel, IN (US); Francisco Alcides VALENZUELA, Indianapolis, IN (US); Yan WANG, Carmel, IN (US); Lin ZHANG, Carmel, IN (US)

(21) Appl. No.: 18/489,919

(22) Filed: Oct. 19, 2023

Related U.S. Application Data
(60) Provisional application No. 63/418,048, filed on Oct. 21, 2022.

(51) Int. Cl. C07K 14/58; A61K 47/54; A61P 9/04

(52) U.S. Cl. CPC C07K 14/58; A61K 47/542; A61P 9/04; A61K 38/00

(57) ABSTRACT

The present invention relates to Atrial Natriuretic Peptide (ANP) polypeptides and methods of treatment with ANP polypeptides.

### CROSS REFERENCE TO RELATED APPLICATIONS

This application claims priority to U.S. Provisional Application 63/418,048 filed Oct. 21, 2022, the content of which is incorporated herein by reference in its entirety.

### REFERENCE TO SEQUENCE LISTING SUBMITTED ELECTRONICALLY

The disclosure is being filed along with a Sequence Listing in ST.26 XML format. The Sequence Listing is provided as a file titled “30262_US_seqlisting.xml” created 3 Oct. 2023 and is 410.8 KB in size. The Sequence Listing information in the ST.26 XML format is incorporated herein by reference in its entirety.

### FIELD OF THE INVENTION

This disclosure relates generally to biology and medicine, and more particularly it relates to peptides that are natriuretic peptide analogs, especially long-acting atrial natriuretic peptide (ANP) polypeptides, that bind to natriuretic peptide receptors, such as the NPR-A, thereby functioning as NPR-A agonists and exhibit improved stability. The disclosure further relates to compositions including the same and their use in treating cardiovascular conditions, diseases or disorders.

### BACKGROUND

There is an unmet medical need for new and improved treatments for Heart Failure (HF). Currently available therapies are intended to slow down disease progression and improve symptoms, and rely on hemodynamic changes to reduce the workload of the failing heart. These therapies include agents intended to: (a) reduce heart rate, such as beta blockers and Hyperpolarization-activated cyclic nucleotide-gated (HCN) channel blockers such as ivabradine; (b) reduce blood pressure, such as angiotensin-converting enzyme (ACE) inhibitors, angiotensin II receptor blockers (ARB), mineralocorticoid receptor antagonists (MRA), and ARB and Neprilysin (NEP) inhibitor combination (sacubitril/valsartan (ENTRESTO®)); and/or (c) treat or prevent volume overload, such as diuretics and MRA. These treatments, however, do not directly treat the heart, and have practical limitations, such as requiring dose titration and monitoring for hypotension. In addition, even with these existing treatment options available, all HF patients, even those who are mildly symptomatic are at high risk of dying. See, e.g., Ahmed A, A propensity matched study of New York Heart Association class and natural history end points in heart failure, AM. J. CARDIOL. 2007; 99(4):549-553. Thus, new and improved HF treatments are needed.

Natriuretic peptides (NPs) are a class of endogenous hormones which confer cardiovascular protection through regulation of body fluid homeostasis. They include four structurally related peptide hormones: Atrial Natriuretic Peptide (ANP), Brain Natriuretic Peptide (BNP), C-type Natriuretic Peptide (CNP) and Dendroaspis Natriuretic Peptide (DNP). Three subtypes of natriuretic peptide receptors (NPR) have been described and include NPR-A, NPR-B and NPR-C.

Wild-type human ANP is a 28 amino acid peptide having a 17 amino acid loop formed by an intramolecular disulfide linkage between two cysteine residues present at positions 7 and 23. It is a cardiac hormone that is part of the body's natural defense against hypoxia and pathological cardiac wall stress. ANP is released in response to myocardial wall stress and elicits natriuretic, diuretic, and vasodilatory effects. ANP acts through the NPR-A to activate the pGC-cGMP pathway and increase intracellular cGMP levels. NPR-A agonists have direct anti-hypertrophic and anti-fibrotic effects in the heart, improve lung function, and can have beneficial effects on glucose metabolism and energy metabolism. ANP treatment can translate into improvements in cardiac filling pressures, promote beneficial cardiac remodeling and improve diastolic function, and exert cardioprotective effects in the heart, vasculature, lungs and kidneys.

However, wild-type ANP has a rapid blood circulation clearance, which may be attributed to its binding to natriuretic peptide receptor C (NPR-C) with subsequent internalization and lysosomal proteolysis, proteolytic cleavage by endopeptidases and renal secretion. Human ANP has an in vivo half-life of only several minutes. Urodilatin, a naturally occurring amino terminal extended form of ANP is more resistant to enzymatic degradation, yet also has an in vivo half-life of only about 6 min. Polypeptides with such short half-life require administration by continuous intravenous infusion, typically in a hospital or other medical care facility, which often results in inconvenience for individuals receiving the polypeptide and in short-term efficacy, typically in a hospital or other medical care facility. Short-term intravenous infusion of recombinant ANP (carperitide) has been approved in Japan and demonstrated some acute benefits. However, short-term infusions for about 48 h showed no long-term outcome benefits.

Several peptide half-life extension technologies exist, for example, peptide conjugation to a fatty acid moiety, to recombinant human serum albumin (rHSA) or bovine serum albumin (BSA), to a pharmaceutically acceptable polymer, such as polymeric sequence of amino acids (XTEN), to unsulfated heparin-like carbohydrate polymer (HEP) or hydroxyl ethyl starch (HES), to a llama heavy-chain antibody fragments (VHH), pegylation, and Fc conjugation, (see e.g. Sleep, D. Epert Opin Drug Del (2015) 12, 793-812; Podust V N et. al. J Control. Release, 2015; ePUB; Hey, T. et. al. in: R. Kontermann (Ed.), Therapeutic Proteins: Strategies to Modulate their Plasma Half-Lives, Wiley-VCH Verlag Gmbh & Co. KGaA, Weinheim, Germany, 2012, pp 117-140; DeAngelis, P L, Drug Dev Delivery (2013) January, Dec. 31, 2012.

Efforts have been made to prepare ANP analogs and derivatives that mimic the biological activity of native ANP and/or have improved stability. For example, EP465097; U.S. Pat. Nos. 4,607,023; 5,212,286; 5,434,133; 6,525,022; 8,058,242; 9,193,777; 10,947,289; 11,312,758; WO 1988/03537; WO 1998/45329; WO 2004/011498; and WO 2018/175534 describe various ANP analogs and derivatives with greater stability. U.S. Pat. No. 5,204,328 describes ANP analogs containing N-alkylated amino acids to protect the peptide from enzymatic degradation. U.S. Pat. No. 6,525,022 describe ANP analogs that have equal binding affinity for NPR-A but decreased affinity for NPR-C. WO 1998/45329 describes ANP derivatives in which a lipophilic substituent is linked to the peptide. WO 2004/011498 describes ANP derivatives comprising a reactive entity coupled to the peptide that renders the peptide capable of forming a peptide-blood component conjugate. U.S. Pat. No. 9,193,777 describes ANP analogs that contain a 12 amino acid C-terminus extension based upon a familial ANP gene frameshift mutation. U.S. Pat. No. 10,947,289 describes glyco-modified ANP derivatives in which a sugar substance is linked to the peptide. WO 2008/154226 describes ANP fusion proteins linked to an antibody Fc fragment.

Nevertheless, a need remains for alternative treatment options. There is a need for therapies that improve long-term outcomes, including increased survival and reduced hospitalization rates. There is also a need for therapies that improve cardiac function, with the potential to modify or reverse the disease. There is also a need for therapies which improve quality of life (QoL) in patients with advanced disease. There is also a need for therapeutic agents available for use with sufficiently extended duration of action to allow for dosing as infrequently as once a day, thrice-weekly, twice-weekly or once a week. The present invention seeks to meet one or more of these critical unmet needs.

### SUMMARY OF INVENTION

Provided herein are ANP polypeptides that bind to and agonize NPR-A and have natriuretic, diuretic and vasorelaxant activity. Moreover, the ANP polypeptides described herein have extended duration of action at NPR-A allowing for dosing as infrequently as once-a-day, thrice-weekly, twice-weekly or once-a-week. The ANP polypeptides described herein also exhibit desirable developability profiles making them suitable for use in therapeutic applications. In this manner, the ANP polypeptides described herein can be useful in chronic treatment to lower blood pressure, reduce pathological wall stress and improve adverse cardiac remodeling, as well as have beneficial effects on lung congestion.

Thus, the present disclosure also provides methods of using ANP polypeptides to treat or prevent cardiovascular disease (CVD) and related conditions, including in particular Heart Failure (HF). Preferred ANP polypeptides and methods of the present invention reduce the risk of CV-related death or HF-related hospitalization, reduce the risk of myocardial infarction (MI) or stroke, reduce the probability of a need for left ventricular assist device (LVAD) or cardiac transplant, improve cardiac function and structure, and/or improve the symptoms and physical limitations associated with HF, leading to improvements in QoL.

In one embodiment, provided herein is a polypeptide of Formula I comprising:

(SEQ ID NO: 3)
X₁X₂X₃RSSCFX₉X₁₀X₁₁IX₁₃RIGX₁₇X₁₈SGLGCPSX₂₆RX₂₈X₂₉,

wherein:
X₁ is absent, S or E,
X₂ is absent, L, K, 4-Pal, H or E,
X₃ is absent, R, β-Ala, P, K, E or G,
X₉ is G, 4-Pal or H,
X₁₀ is G, K, R or Dap,
X₁₁ is R, K, G or Dap,
X₁₃ is D or G,
X₁₇ is A, H, Dap, K, R or Om,
X₁₈ is Q, Y or 4-Pal,
X₂₆ is F or L,
X₂₈ is Y, H or 4-Pal, and
X₂₉ is either absent or selected from

GGP,
(SEQ ID NO: 4)
SGAPPPE,
(SEQ ID NO: 5)
KITAKEDE,
(SEQ ID NO: 6)
GPSSGAPPPE,
(SEQ ID NO: 7)
GPSSGAPPPS,
(SEQ ID NO: 8)
GGSSGAPPPS,
(SEQ ID NO: 9)
GGPSSGAPPPS,
(SEQ ID NO: 10)
KGPSSGAPPPS,
(SEQ ID NO: 11)
GGKSSGAPPPS,
(SEQ ID NO: 12)
GGPPS-Aib-KPPPK,
(SEQ ID NO: 13)
GSPSSGAPPPS,
(SEQ ID NO: 14)
RITAREDKQGYA,
(SEQ ID NO: 15)
RITAREDKQGEA,
(SEQ ID NO: 16)
GSPSSGAPPPS-PEG24-G,
(SEQ ID NO: 17)
SGSPSSGAPPPSG,
(SEQ ID NO: 18)
GGESSGEPPPSEE,
(SEQ ID NO: 19)
GSGSPSSGAPPPSG,
and
(SEQ ID NO: 20)
SGSPSSGAPPPSEEEG

and the C-terminal amino acid is optionally amidated,
or a pharmaceutically acceptable salt thereof.

In some embodiments, the polypeptide contains a disulfide linkage between the cysteines present at positions 7 and 23 (C7 and C23). In some embodiments, the polypeptide contains a thioacetal linkage between the cysteines present at positions 7 and 23 (C7 and C23).

In another embodiment, a polypeptide of Formula I, or a pharmaceutically acceptable salt thereof, is conjugated to a fatty acid. For instance, in some embodiments, the polypeptide of Formula I, or a pharmaceutically acceptable salt thereof, further comprises a fatty acid conjugated to the amino acid present at the N terminus of the polypeptide and comprises a basic structure from an amino-terminus (N-terminus) to a carboxy-terminus (C-terminus) of Formula II:

(SEQ ID NO: 21)
fatty acid-Z1-Z2-Z3-X₁X₂X₃RSSCFX₉X₁₀X₁₁IDRI
GX₁₇X₁₈SGLGCX₂₄SX₂₆RX₂₈X₂₉,

wherein the fatty acid is a C₁₆-C₂₆ fatty acid and is conjugated to the amino acid present at the N terminus of the polypeptide through a structure Z1-Z2-Z3, wherein
Z1 comprises an amino acid selected from γGlu, E and β-Ala,
Z2 is either absent or comprises a four to ten amino acid sequence comprising amino acids independently selected from E, K, G, P, A and S, and
Z3 is either absent or comprises a polyethylene glycol (PEG) or a (2-[2-(2-amino-ethoxy)-ethoxy]-acetyl) moiety.

In some embodiments, Z1 is an amino acid selected from γGu, E and β-Ala.

In some embodiments, Z2 is selected from APPSG, (EK)_{b}G, (EP)_{b}G, K(EK)_{c}G, and (EK)_{c}E, wherein b is 2, 3 or 4 and c is 1, 2, 3 or 4. For example, in some embodiments, Z2 is EKEKEKG (SEQ ID NO:22), EPEPEPG (SEQ ID NO:23), APPSG (SEQ ID NO:24), KEKEKG (SEQ ID NO:25) or EKEKEKE (SEQ ID NO:26).

In some embodiments, Z3 is selected from (polyethylene glycol)ₘ wherein m is a whole number selected from 10 to 30 and ((2-[2-(2-amino-ethoxy)-ethoxy]-acetyl))ₙ wherein n is selected from 2 to 10. For example, in some embodiments, Z3 is (polyethylene glycol)₁₂ or (polyethylene glycol)₂₄ or ((2-[2-(2-amino-ethoxy)-ethoxy]-acetyl))₄ or (2-[2-(2-amino-ethoxy)-ethoxy]-acetyl))₆ or (2-[2-(2-amino-ethoxy)-ethoxy]-acetyl))₈.

In another embodiment, a pharmaceutical composition is provided that includes a polypeptide, or a pharmaceutically acceptable salt thereof, as described herein and a pharmaceutically acceptable carrier, diluent or excipient.

In another embodiment, provided herein is a method for using a polypeptide or a pharmaceutically acceptable salt thereof described herein to treat or prevent a cardiovascular disease (CVD) and related conditions. Such methods can include at least a step of administering to an individual in need thereof an effective amount of a polypeptide described herein, or a pharmaceutically acceptable salt thereof. In some instances, the CVD is heart failure (HF), in particular it is Heart Failure with preserved Ejection Factor (HfpEF).

In another embodiment, a polypeptide, or a pharmaceutically acceptable salt thereof, as described herein is provided for use in therapy.

In another embodiment, a polypeptide, or a pharmaceutically acceptable salt thereof, as described herein is provided for use in treating or preventing a CVD. In some instances, the CVD is HF, in particular it is HfpEF.

In another embodiment, a polypeptide, or a pharmaceutically acceptable salt thereof, as described herein is provided for use in manufacturing a medicament for treating or preventing a CVD. In some instances, the CVD is HF, in particular it is HfpEF.

### DETAILED DESCRIPTION

Unless defined otherwise, all technical and scientific terms used herein have the same meaning as commonly understood by one of skill in the art to which the disclosure pertains. Although any methods and materials similar to or equivalent to those described herein can be used in the practice or testing of the ANP polypeptides, pharmaceutical compositions, and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and in reference to one or more of the ANP receptors, “activity,” “activate,” “activating” and the like means a capacity of a compound, such as ANP polypeptides described herein, to bind to and induce a response at the receptor(s), as measured using assays known in the art, such as the in vitro assays described below.

As used herein, “ANP polypeptide” means an ANP analog having structural similarities with, but some differences from, naturally occurring ANP, especially rat ANP (SEQ ID NO:1) or human ANP (SEQ ID NO:2). The ANP polypeptides described herein include amino acid sequences resulting in the polypeptides having affinity for and activity at the NPR-A receptor. The term “ANP polypeptide” also includes acylated or otherwise derivatized ANP analog.

As used herein, “conservative substitution” means a variant of a reference peptide or polypeptide that is identical to the reference molecule, except for having one or more conservative amino acid substitutions in its amino acid sequence. In general, a conservatively modified variant includes an amino acid sequence that is at least about 70%, 75%, 80%, 85%, 90%, 91%, 92%, 93%, 94%, 95%, 96%, 97%, 98% or 99% identical to a reference amino acid sequence. More specifically, a conservative substitution refers to substitution of an amino acid with an amino acid having similar characteristics (e.g., charge, side-chain size, hydrophobicity/hydrophilicity, backbone conformation and rigidity, etc.) and having minimal impact on the biological activity of the resulting substituted peptide or polypeptide. Conservative substitutions of functionally similar amino acids are well known in the art and thus need not be exhaustively described herein.

As used herein, a “C₁₆-C₂₆ fatty acid” means a carboxylic acid having between 16 and 26 carbon atoms. The C₁₆-C₂₆ fatty acid suitable for use herein can be a linear fatty acid or a branched fatty acid. The linear C₁₆-C₂₆ fatty acid suitable for use herein can be a saturated monoacid or a saturated diacid. As used herein, “saturated” means the fatty acid contains no carbon-carbon double or triple bonds.

As used herein, “effective amount” means an amount, concentration or dose of one or more ANP polypeptides described herein, or a pharmaceutically acceptable salt thereof which, upon single or multiple dose administration to an individual in need thereof, provides a desired effect in such an individual under diagnosis or treatment. An effective amount is also one in which any toxic or detrimental effects of the polypeptide are outweighed by the therapeutically beneficial effects. An effective amount can be determined by one of skill in the art through the use of known techniques and by observing results obtained under analogous circumstances. In determining the effective amount for an individual, a number of factors are considered including, but not limited to, the species of mammal; its size, age and general health; the specific disease or disorder involved; the degree of or involvement of or the severity of the disease or disorder; the response of the individual patient; the particular ANP polypeptide administered; the mode of administration; the bioavailability characteristics of the preparation administered; the dose regimen selected; the use of concomitant medication; and other relevant circumstances.

As used herein, “extended duration of action” means that binding affinity and activity for an ANP polypeptide continues for a period of time greater than native human ANP polypeptide, allowing for dosing at least as infrequently as once daily, thrice-weekly, twice-weekly, once-weekly, or less than once weekly such as biweekly (once in two weeks) or even monthly. The time action profile of the ANP polypeptide may be measured using known pharmacokinetic test methods such as those utilized in the examples below.

As used herein, “half-life” or “t½” means a time it takes for one-half of a quantity of a compound, such as native ANP or an ANP polypeptide herein, to be removed from a fluid or other physiological space such as serum or plasma of an individual by biological processes. Alternatively, t½ also can mean a time it takes for a quantity of such a compound to lose one-half of its pharmacological, physiological or radiological activity.

As used herein, “half-maximal effective concentration” or “EC₅₀” means a concentration of polypeptide that results in 50% activation/stimulation of an assay endpoint, such as a dose-response curve (e.g., cGMP signaling pathway).

As used herein, “in combination with” means administering at least one of the ANP polypeptides herein either simultaneously, sequentially or in a single combined formulation with one or more additional therapeutic agents.

As used herein, “individual in need thereof” means a mammal, such as a human, with a condition, disease, disorder or symptom requiring treatment or therapy, including for example, those listed herein.

As used herein, “long-acting” means that binding affinity and activity of an ANP polypeptide herein continues for a period of time greater than native, human ANP (SEQ ID NO:2), allowing for dosing at least as infrequently as once daily or even thrice-weekly, twice-weekly, or once-weekly. The time action profile of the ANP polypeptides may be measured using known pharmacokinetic test methods such as those described in the Examples below.

As used herein, the term “pharmaceutically acceptable salt” refers to derivatives of the polypeptides herein, where a polypeptide herein is modified by making acid or base salts thereof. Pharmaceutically acceptable salts, and processes for preparing the same, are well known in the art (see, e.g., Remington: The Science and Practice of Pharmacy, L. V. Allen, Ed., 22nd Edition, Pharmaceutical Press, 2012). By way of example, pharmaceutically acceptable salts include, but are not limited to, mineral or organic acid salts of basic residues such as amines, or alkali or organic salts of acidic residues such as carboxylic acids. Pharmaceutically acceptable salts include the conventional non-toxic salts or the quaternary ammonium salts of a polypeptide herein formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically acceptable salts are those forms of a polypeptide herein, suitable for use in contact with the tissues of human beings and animals without excessive toxicity, irritation, allergic response, or other problem or complication, commensurate with a reasonable benefit/risk ratio. Pharmaceutically acceptable salt forms of a polypeptide herein can be synthesized to contain a basic or acidic moiety by conventional chemical methods. Generally, such salts are, for example, prepared by reacting the free acid or base forms of the polypeptide with a stoichiometric amount of the appropriate base or acid in water or in an organic solvent, or in a mixture of the two; generally, nonaqueous media like ether, ethyl acetate, ethanol, isopropanol, or acetonitrile are preferred (see, e.g., Stahl et al., “Handbook of Pharmaceutical Salts: Properties, Selection and Use” (Wiley-VCH 2nd ed. 2011)).

The term, “pharmaceutical composition,” as used herein, refers to a composition having an effective amount of one or more peptides herein in combination with other chemical components, such as binders, carriers, diluents, lubricants, pharmaceutical flow agents, and/or other excipients, especially a pharmaceutically acceptable carrier.

As used herein, “polypeptide” or “peptide” means a polymer of amino acid residues comprising two (2) or more amino acids and/or amino acid derivatives which, in general, are linked via peptide bonds. The term applies to polymers comprising naturally occurring amino acids and polymers comprising one or more non-naturally occurring amino acids. Embodiments may include modifications or amino acid derivatives, including post-translational modifications such as, phosphorylation, hydroxylation, sulfonation, palmitoylation, glycosylation and disulfide formation.

As used herein, “treat,” “treating,” “to treat” and the like mean managing and caring for an individual having a condition, disease, disorder or symptom for which an ANP polypeptide administration is indicated for the purpose of attenuating, restraining, slowing, stopping or reversing the progression or severity of the condition, disease, disorder or symptom. Treating includes administering an ANP polypeptide herein or composition containing an ANP polypeptide herein to the individual to prevent the onset of symptoms or complications, alleviating the symptoms or complications, or eliminating the condition, disease, disorder or symptom. Treating includes administering an ANP polypeptide or composition containing an ANP polypeptide herein to the individual to result in such as, for example, increased angiogenesis, increased vascular compliance, increased glomerular filtration rate, decreased blood pressure, decreased (or prevented) inflammation and/or reduced (or prevented) fibrosis in the heart, kidney, liver or lung).

In one embodiment, provided herein is a polypeptide of Formula I:

(SEQ ID NO: 3)
X₁X₂X₃RSSCFX₉X₁₀X₁₁IX₁₃RIGX₁₇X₁₈SGLGCPSX₂₆RX₂₈X₂₉,

wherein:
X₁ is absent, S or E,
X₂ is absent, L, K, 4-Pal, H or E,
X₃ is absent, R, β-Ala, P, K, E or G,
X₉ is G, 4-Pal or H,
X₁₀ is G, K, R or Dap,
X₁₁ is R, K, G or Dap,
X₁₃ is D or G,
X₁₇ is A, H, Dap, K, R or Om,
X₁₈ is Q, Y or 4-Pal,
X₂₆ is F or L,
X₂₈ is Y, H or 4-Pal, and
X₂₉ is either absent or selected from

GGP,
(SEQ ID NO: 4)
SGAPPPE,
(SEQ ID NO: 5)
KITAKEDE,
(SEQ ID NO: 6)
GPSSGAPPPE,
(SEQ ID NO: 7)
GPSSGAPPPS,
(SEQ ID NO: 8)
GGSSGAPPPS,
(SEQ ID NO: 9)
GGPSSGAPPPS,
(SEQ ID NO: 10)
KGPSSGAPPPS,
(SEQ ID NO: 11)
GGKSSGAPPPS,
(SEQ ID NO: 12)
GGPPS-Aib-KPPPK,
(SEQ ID NO: 13)
GSPSSGAPPPS,
(SEQ ID NO: 14)
RITAREDKQGYA,
(SEQ ID NO: 15)
RITAREDKQGEA,
(SEQ ID NO: 16)
GSPSSGAPPPS-PEG24-G,
(SEQ ID NO: 17)
SGSPSSGAPPPSG,
(SEQ ID NO: 18)
GGESSGEPPPSEE,
(SEQ ID NO: 19)
GSGSPSSGAPPPSG,
and
(SEQ ID NO: 20)
SGSPSSGAPPPSEEEG

and the C-terminal amino acid is optionally amidated,
or a pharmaceutically acceptable salt thereof.

The structural features described herein result in the polypeptides having sufficient activity at NPR-A, and also result in the polypeptides having many other beneficial attributes relevant to their developability as therapeutic treatments, including for improving solubility of the analogs in aqueous solutions, improving chemical and physical formulation stability, extending the pharmacokinetic profile, and minimizing potential for immunogenicity.

In some embodiments, X₁ is selected from S and E. In some embodiments, X₂ is selected from K and 4-Pal. In some embodiments, X₃ is selected from R, β-Ala, P and K. In some embodiments, X₉ is G, 4-Pal or H. In some embodiments, X₁₀ is selected from G, K, R and Dap. In some embodiments, X₁₁ is selected from R and K. In some embodiments, X₁₃ is selected from D and G. In some embodiments, X₁₇ is H, K, R, Dap or Om. In some embodiments, X₁₈ is selected from Q and Y. In some embodiments, X₂₈ is F or L. In some embodiments, X₂₈ is H or 4-Pal. In some embodiments, X₂₉ is absent or selected from

(SEQ ID NO: 9)
GGPSSGAPPPS,
(SEQ ID NO: 11)
GGKSSGAPPPS
and
(SEQ ID NO: 13)
GSPSSGAPPPS

In some embodiments, X₁ is selected from S and E; X₂ is selected from K and 4-Pal; X₃ is selected from R, β-Ala, P and K; X₉ is G, 4-Pal or H; X₁₀ is selected from G, K, R and Dap; X₁₁ is selected from R and K; X₁₃ is selected from D and G; X₁₇ is H, K, R, Dap or Om; X₁₈ is selected from Q and Y; X₂₆ is F or L; X₂₈ is H or 4-Pal; and X₂₉ is absent or selected from GGPSSGAPPPS (SEQ ID NO:9), GGKSSGAPPPS (SEQ ID NO:11) and GSPSSGAPPPS (SEQ ID NO:13).

In some embodiments, X₁ is selected from S and E. In some embodiments, X₂ is selected from K and 4-Pal. In some embodiments, X₃ is selected from R, β-Ala and K. In some embodiments, X₉ is G. In some embodiments, X₁₀ is selected from G and K. In some embodiments, X₁₁ is selected from R and K. In some embodiments, X₁₃ is selected from D and G. In some embodiments, X₁₇ is H. In some embodiments, X₁₈ is selected from Q and Y. In some embodiments, X₂₆ is F. In some embodiments, X₂₈ is H. In some embodiments, X₂₉ is absent or selected from GGPSSGAPPPS (SEQ ID NO:9), GGKSSGAPPPS (SEQ ID NO:11) and GSPSSGAPPPS (SEQ ID NO:13).

In some embodiments, X₁ is selected from S and E, X₂ is selected from K and 4-Pal, X₃ is selected from R, β-Ala and K, X₉ is G, X₁₀ is selected from G and K, X₁₁ is selected from R and K, X₁₃ is selected from D and G, X₁₇ is H, X₁₈ is selected from Q and Y, X₂₆ is F, X₂₈ is H, and X₂₉ is absent or selected from GGPSSGAPPPS (SEQ ID NO:9), GGKSSGAPPPS (SEQ ID NO:11) and GSPSSGAPPPS (SEQ ID NO:13).

In some embodiments, the polypeptide contains a disulfide linkage between the cysteines present at positions 7 and 23 (C7 and C23) of SEQ ID NO:3. In some embodiments, the polypeptide contains a thioacetal linkage between the cysteines present at positions 7 and 23 (C7 and C23).

In some embodiments, a polypeptide described herein is conjugated to a fatty acid.

In another embodiment, a polypeptide of Formula I, or a pharmaceutically acceptable salt thereof, is conjugated to a fatty acid. For instance, in some embodiments, it further comprises a fatty acid conjugated to the amino acid present at the N terminus of the polypeptide, and comprises a basic structure from an amino-terminus (N-terminus) to a carboxy-terminus (C-terminus) of Formula II:

(SEQ ID NO: 21)
fatty acid-Z1-Z2-Z3-X₁X₂X₃RSSCFX₉X₁₀
X₁₁IDRIGX₁₇X₁₈SGLGCX₂₄SX₂₆RX₂₈X₂₉,

wherein the fatty acid is a C₁₆-C₂₆ fatty acid and is conjugated to the amino acid present at the N terminus of the polypeptide through a structure Z1-Z2-Z3,
wherein Z1 comprises an amino acid selected from γGlu, E and β-Ala,
Z2 is either absent or comprises a four to ten amino acid sequence comprising amino acids independently selected from E, K, G, P, A and S, and
Z3 is either absent or comprises a polyethylene glycol or a (2-[2-(2-amino-ethoxy)-ethoxy]-acetyl) moiety.

The polypeptides of Formula II described herein include a fatty acid moiety conjugated, for example by way of a linker comprising a structure of Z1 or Z1-Z2 or Z1-Z3 or Z1-Z2-Z3, to the amino acid present at the N terminus of SEQ ID NO:3. Such a conjugation is sometimes referred to as acylation. In embodiments, where X1 is absent, the fatty acid, for example by way of a linker comprising a structure of Z1 or Z1-Z2 or Z1-Z3 or Z1-Z2-Z3, is conjugated to the amino acid present at position X2 of SEQ ID NO:3. In embodiments, where both X1 and X2 are absent, the fatty acid is conjugated to the amino acid present at position X3 of SEQ ID NO:3 (for example by way of a linker comprising a structure of Z1 or Z1-Z2 or Z1-Z3 or Z1-Z2-Z3). In embodiments, where both X1, X2 and X3 are absent, the fatty acid is conjugated to the amino acid present at position X4 of SEQ ID NO:3 (for example by way of a linker comprising a structure of Z1 or Z1-Z2 or Z1-Z3 or Z1-Z2-Z3). The fatty acid, and in certain embodiments the linker, act as albumin binders, and provide a potential to generate long-acting polypeptides.

The polypeptides described herein utilize a C₁₆-C₂₆ fatty acid that can be chemically conjugated to the functional group of an amino acid either by a direct bond or by a linker. The length and composition of the fatty acid impacts half-life of the polypeptides, their potency in in vivo animal models, and their solubility and stability. Conjugation to a C₁₆-C₂₆ fatty acid results in ANP polypeptides that exhibit desirable half-life, desirable potency in in vivo animal models, and desirable solubility and stability characteristics.

In some embodiments, the fatty acid is a C₁₆-C₂₂ saturated fatty monoacid or diacid. Examples of saturated C₁₆-C₂₂ fatty acids for use herein include, but are not limited to, palmitic acid (hexadecanoic acid) (C₁₆ monoacid), hexadecanedioic acid (C₁₆ diacid), margaric acid (heptadecanoic acid) (C₁₇ monoacid), heptadecanedioic acid (C₁₇ diacid), stearic acid (C₁₈ monoacid), octadecanedioic acid (C₁₈ diacid), nonadecylic acid (nonadecanoic acid)(C₁₉ monoacid), nonadecanedioic acid (C₁₉ diacid), arachadic acid (eicosanoic acid)(C₂₀ monoacid), eicosanedioic acid (C₂₀ diacid), heneicosylic acid (heneicosanoic acid)(C₂₁ monoacid), heneicosanedioic acid (C₂₁ diacid), behenic acid (docosanoic acid)(C₂₂ monoacid), docosanedioic acid (C₂₂ diacid), including branched and substituted derivatives thereof.

In certain instances, the C₁₆-C₂₂ fatty acid can be a saturated C₁₆ monoacid, a saturated C₁₆ diacid, a saturated C₁₈ monoacid, a saturated C₁₈ diacid, a saturated C₂₀ monoacid, a saturated C₂₀ diacid, and branched and substituted derivatives thereof.

In some embodiments, the linker can have a structure of Z1-Z2-Z3, wherein Z1 comprises an amino acid selected from γGlu, E and β-Ala; Z2 is either absent or comprises a four to ten amino acid sequence comprising amino acids independently selected from E, K, G, P, A and S; and Z3 is either absent or comprises a polyethylene glycol or a (2-[2-(2-amino-ethoxy)-ethoxy]-acetyl) moiety as shown below.

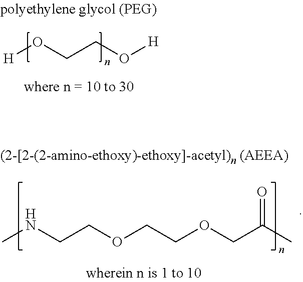

Accordingly, in some embodiments, the fatty acid is attached to Z1, and Z1 is attached to the peptide of Formula I either directly or via Z2 or via Z3 or via Z2-Z3.

In some instances, Z1 is an amino acid selected from γGlu, E and β-Ala, or a dipeptide such as γGlu-γGlu or E-γGlu, or a tripeptide such as γGlu-γGlu-γGlu. In some embodiments, Z1 is γGlu or β-Ala. In some embodiments, Z1 is γGlu.

In some embodiments, the fatty acid is attached to Z1, Z1 is attached to Z2 and Z2 is attached to a peptide of Formula I either directly or via Z3. In some embodiments, Z2 is selected from APPSG, (EK)_{b}G, (EP)_{b}G, K(EK)_{c}G, and (EK)_{c}E, wherein b is 2, 3 or 4 and c is 1, 2, 3 or 4. For example, Z2 may be (EK)₃G i.e. EKEKEKG, (EP)₃G i.e. EPEPEPG, K(EK)₂G i.e. KEKEKG or (EK)₃E i.e. EKEKEKE. In some embodiments, Z2 is EKEKEKG.

In some embodiments, the fatty acid is attached to Z1, Z1 is attached to Z2, Z2 is attached to Z3, and Z3 is attached to a peptide of Formula I. In some embodiments, Z3 is selected from (polyethylene glycol)ₘ wherein m is a whole number selected from 10 to 30 and ((2-[2-(2-amino-ethoxy)-ethoxy]-acetyl))ₙ wherein n is selected from 2 to 10. For example, in some embodiments, Z3 is (polyethylene glycol)₁₂ or (polyethylene glycol)₂₄ or ((2-[2-(2-amino-ethoxy)-ethoxy]-acetyl))₄ or -(2-[2-(2-amino-ethoxy)-ethoxy]-acetyl))₆ or (2-[2-(2-amino-ethoxy)-ethoxy]-acetyl))₈.

In some embodiments, the fatty acid is a branched C₂₅ triacid having the following structure (also referred to herein as Bifurcated Fatty Acid or “BFA”):

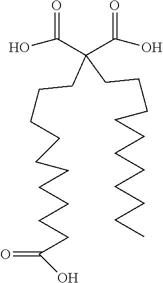 [SMILES: CCCCCCCCCCCC(CCCCCCCCCCC(=O)O)(C(=O)O)C(=O)O]

The BFA exists in two enantiomeric forms.

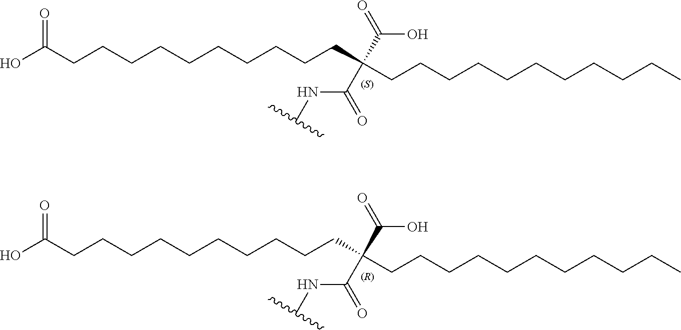

It was surprisingly discovered that a purified enantiomer (EN2) of the BFA provides tighter binding to albumin as compared to the other enantiomer (EN1) or the racemic mixture, and results in a more desirable PK profile in rats. The isolation of purified EN2 (Preparation 8B) from the racemic mixture (Preparation 8) is described below. It was further discovered that for conserving the stability of the enantiomerically pure BFA during the coupling step to the peptide, it is essential to attach it to a Z1, wherein the Z1 comprises β-Ala or γGlu or E.

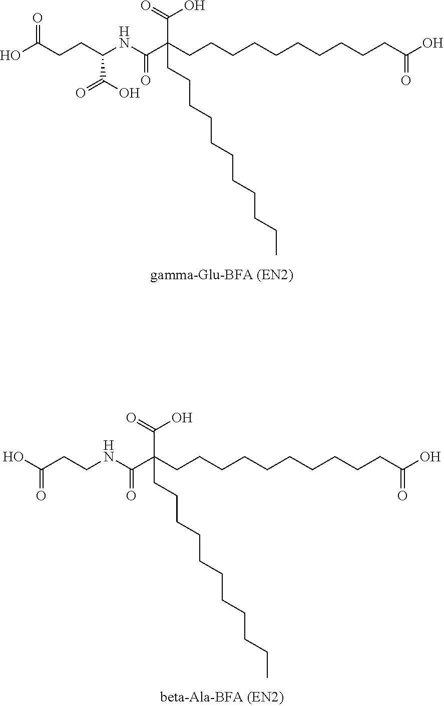 [SMILES: CCCCCCCCCCCC(CCCCCCCCCCC(=O)O)(C(=O)O)C(=O)N[C@@H](CCC(=O)O)C(=O)O ; CCCCCCCCCCCC(CCCCCCCCCCC(=O)O)(C(=O)O)C(=O)NCCC(=O)O]

Thus, in one aspect, the present invention includes a purified enantiomer EN2 of the BFA, attached to β-Ala or to γGlu or to E. Accordingly in one embodiment, included herein is a structure in which a purified enantiomer EN2 of the BFA is attached to β-Ala. In another embodiment, included herein is a structure in which a purified enantiomer EN2 of the BFA is attached to γGlu. In another embodiment, included herein is a structure in which a purified enantiomer EN2 of the BFA is attached to E.

In some embodiments, the polypeptides of Formula II comprise a purified enantiomer EN2 of the BFA attached to β-Ala. In some embodiments, the polypeptides of Formula II comprise a purified enantiomer EN2 of the BFA attached to γGlu. In some embodiments, the polypeptides of Formula II comprise a purified enantiomer EN2 of the BFA attached to E, E-γGlu, γGlu-γGlu or γGlu-γGlu-γGlu. In some embodiments, Z2 is selected from EKEKEKG, KEKEKG and EKEKEKE. In some embodiments, Z3 is selected from (polyethylene glycol)₁₂ and (polyethylene glycol)₂₄.

The amino acid sequences of ANP polypeptides described herein incorporate naturally occurring amino acids, typically depicted herein using standard one letter codes (e.g., L=leucine), as well as certain other unnatural amino acids, such as 3-(4-Pyridyl)-L-alanine (4Pal), L-Omithine (Orn), L-2,3-diaminopropionic acid (Dap) and β-Ala. The structures of the non-natural amino acids appear below:

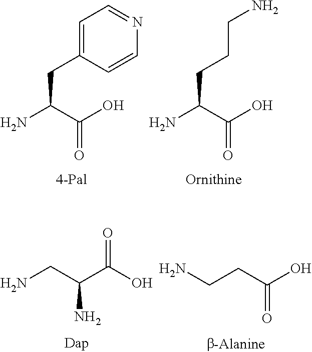

As noted above, the ANP polypeptides described herein have structural similarities to, but many structural differences, from any of the native human natriuretic peptides. For example, when compared to native human ANP (SEQ ID NO:2), the ANP polypeptides described herein include modifications at one or more of positions 1, 2, 3, 9, 10, 11, 12, 13, 17, 18, 24, 26, 28 and 29. In some instances, ANP polypeptides described herein include modifications at each of the positions 1, 2, 3, 9, 10, 11, 12, 13, 17, 18, 24, 26, 28 and 29. In addition, in some embodiments, the ANP polypeptides contain a thioacetal (S-CH2-S) linkage between cysteines present at positions 7 and 23.

In some embodiments, the ANP polypeptides described herein include the following amino acid modifications: S or E at position 1; K or 4-Pal at position 2; R, β-Ala, P or K at position 3; G, 4-Pal or H at position 9; G, K, R or Dap at position 10; R or K at position 11; D or G at position 13; H, K, R, Dap or Om at position 17; Q or Y at position 18; F or L at position 26; H or 4-Pal at position 28; and attachments at positions 29-39 with an amino acid sequence selected from GGPSSGAPPPS (SEQ ID NO:9), GGKSSGAPPPS (SEQ ID NO:11) and GSPSSGAPPPS (SEQ ID NO:13); and conjugation to the amino acid at position 1 with a C₁₆ to C₂₂ fatty acid, optionally through the use of a linker comprising the structure Z1-Z2-Z3.

In certain instances, the ANP polypeptides described herein include the following amino acid modifications: S or E at position 1; K or 4-Pal at position 2; R, β-Ala or K at position 3; G at position 9; G or K at position 10; R or K at position 11; I at position 12; D or G at position 13; H at position 17; Q or Y at position 18; P at position 24; F at position 26; H at position 28; and attachments at positions 29-39 with an amino acid sequence selected from GGPSSGAPPPS (SEQ ID NO:9), GGKSSGAPPPS (SEQ ID NO:11) and GSPSSGAPPPS (SEQ ID NO:13); and conjugation to the amino acid at position 1 with a C₁₆ to C₂₂ fatty acid, optionally through the use of a linker comprising the structure Z1-Z2-Z3.

In some embodiments, the ANP polypeptides described herein comprise a sequence selected from any one of SEQ ID NO:28 to 167. In some embodiments, the ANP polypeptides described herein comprise a sequence selected from the group consisting of any one of SEQ ID NO:28 to 167.

In some embodiments, the ANP polypeptides described herein comprise a sequence selected from any one of SEQ ID NO:168 to 172. In some embodiments, the ANP polypeptides described herein comprise a sequence selected from the group consisting of any one of SEQ ID NO:168 to 172.

In some embodiments, the ANP polypeptides described herein comprise a sequence selected from SEQ ID NO:28, 45, 50, 51, 78, 83, 84, 97, 98, 144, 158 and 159. In some embodiments, the ANP polypeptides described herein comprise a sequence selected from the group consisting of SEQ ID NO:28, 45, 50, 51, 78, 83, 84, 97, 98, 144, 158 and 159. For instance, in one embodiment, the ANP polypeptide described herein comprises SEQ ID NO:28. In another embodiment, the ANP polypeptide described herein comprises SEQ ID NO:45. In another embodiment, the ANP polypeptide described herein comprises SEQ ID NO:50. In another embodiment, the ANP polypeptide described herein comprises SEQ ID NO:51. In another embodiment, the ANP polypeptide described herein comprises SEQ ID NO:78. In another embodiment, the ANP polypeptide described herein comprises SEQ ID NO:83. In another embodiment, the ANP polypeptide described herein comprises SEQ ID NO:84. In another embodiment, the ANP polypeptide described herein comprises SEQ ID NO:97. In another embodiment, the ANP polypeptide described herein comprises SEQ ID NO:98. In another embodiment, the ANP polypeptide described herein comprises SEQ ID NO:144. In another embodiment, the ANP polypeptide described herein comprises SEQ ID NO:158. In another embodiment, the ANP polypeptide described herein comprises SEQ ID NO:159.

In certain instances, the ANP polypeptides described herein are amidated. In some embodiments, the ANP polypeptide is an agonist of NPR-A. In addition to the changes described herein, the ANP polypeptides described herein may include one or more additional amino acid modifications, provided, however, that the polypeptides remain capable of binding to and activating NPR-A receptor.

The affinity of the ANP polypeptides described herein for the NPR-A receptor may be measured using techniques known in the art for measuring receptor binding levels, including, for example, those described in the examples below, and is commonly expressed as an inhibitory constant (Ki) value. The activity of the ANP polypeptides described herein at the NPR-A receptor also may be measured using techniques known in the art, including, for example, the in vitro activity assays described below, and is commonly expressed as an EC₅₀ value, which is the concentration of polypeptide causing half-maximal stimulation in a dose response curve.

In further embodiments, provided herein are pharmaceutically acceptable salt forms of the ANP polypeptides. For instance, pharmaceutically acceptable salts for use herein include, but are not limited to, sodium, trifluoroacetate, hydrochloride and/or acetate salts.

In further embodiments, provided herein are pharmaceutical compositions comprising a ANP polypeptide or a pharmaceutically acceptable salt thereof, and at least one of a pharmaceutically acceptable carrier, diluent or excipient.

The ANP polypeptides described herein may be used for treating a variety of conditions, disorders, diseases or symptoms. In particular, methods are provided for treating a cardiovascular condition, disorder or disease or in an individual, where such methods include at least a step of administering to an individual in need of such treatment an effective amount of an ANP polypeptide described herein, or a pharmaceutically acceptable salt thereof, or a pharmaceutical composition comprising them. Exemplary cardiovascular conditions, diseases and disorders include, but are not limited to, acute heart failure, chronic heart failure, Heart Failure with preserved Ejection Factor (HFpEF), Heart Failure with reduced Ejection Factor (HFrEF), atherosclerosis, coronary artery disease, diabetes, stroke, hypercholesterolemia, hypertension, ischemia, vasoconstriction and ventricular hypertrophy, other heart related disorders or conditions such as stroke, hypertension, congestive heart failure, diabetic heart disease, cardio myopathy, diastolic dysfunction vasoconstriction and ventricular hypertrophy. In some embodiments, the heart disease is a condition that is or is related to cardiac senescence and/or diastolic dysfunction due to aging. In some embodiments, the ANP polypeptides described herein are used for treating HFpEF.

Another use of the ANP polypeptides herein is for treating pulmonary conditions, diseases and/or disorders. Exemplary pulmonary conditions, diseases and disorders include, but are not limited to, pulmonary hypertension and chronic obstructive pulmonary disease (COPD).

Another use of the ANP polypeptides herein is for treating renal conditions, diseases and/or disorders. Exemplary renal conditions, diseases and disorders include, but are not limited to, chronic kidney disease and diabetes nephropathy.

Such methods can include selecting an individual having a cardiovascular condition, disease or disorder or who is predisposed to the same. Alternatively, the methods can include selecting an individual having a pulmonary condition, disease or disorder or who is predisposed to the same. Alternatively, the methods can include selecting an individual having a renal condition, disease or disorder or who is predisposed to the same. In certain instances, the methods can include selecting an individual who is diabetic, hypertensive with kidney function impairment and/or obese.

Accordingly, in some embodiments, provided herein is a method for treating a CVD comprising administering to a patient in need thereof, an effective amount of an ANP polypeptide described herein or a pharmaceutically acceptable salt thereof. In some embodiments, the CVD is heart failure. In an embodiment, the CVD is HFpEF.

In some embodiments, provided herein is an ANP polypeptide or a pharmaceutically acceptable salt thereof, for use in therapy.

In some embodiments, provided herein is a use of an ANP polypeptide or a pharmaceutically acceptable salt thereof, in treating a CVD. In some embodiments, the CVD is heart failure. In an embodiment, it is HFpEF.

In some embodiments, provided herein is a use of an ANP polypeptide or a pharmaceutically acceptable salt thereof, in the manufacture of a medicament for treating a CVD. In some embodiments, the CVD is heart failure. In an embodiment, it is HFpEF.

Treatment of heart failure or HFpEF according to the present invention may be reflected in one or more of a variety of measures relevant to heart failure, including, for example: reductions in left ventricular end-diastolic pressure (LVEDP), reductions in the risk of CV death and/or heart failure hospitalization, reductions in the risk of total mortality, reductions in the risk of myocardial infarction (MI), reductions in the risk of stroke, reductions in the risk of need for left ventricular assist device (LVAD) implantation and/or cardiac transplant, improvement in symptoms and physical limitations of heart failure and/or improvement in quality of life (QoL). Certain benefits of treatment according to embodiments of the present invention may be achieved after treatment for at least 1 month. Certain benefits of treatment according to embodiments of the present invention may be achieved after treatment for at least 6 months. Certain benefits of treatment according to embodiments of the present invention may be achieved after treatment for at least 1 year.

In certain embodiments, administration of ANP polypeptides according to the present invention results in significant reductions in LVEDP after 1 year of treatment. In certain embodiments, administration of ANP polypeptides according to the present invention results in a significant reduction in global longitudinal strain (GLS). In certain embodiments, administration of ANP polypeptides according to the present invention results in at least a 3.5% reduction in GLS. In certain embodiments, administration of ANP polypeptides of the present invention results in at least a 15% reduction in risk of CV death and/or HF hospitalization. In certain embodiments, administration of ANP polypeptides of the present invention results in a significant reduction in the risk of one or more of total mortality, MI, stroke, LVAD implantation or cardiac transplant. In certain embodiments, administration of ANP polypeptides of the present invention results in a significant improvement in symptoms and physical limitations of heart failure and/or QoL.

In addition, as noted above, administration of ANP polypeptides according to certain embodiments of the disclosure is capable of providing improvements in heart failure-related measures, such as those described above, without increasing safety risks. Thus, in some embodiments, administration of ANP polypeptides according to the present invention results in no increases in safety risks such as increased hypotension; worsened renal function; electrolyte imbalances; liver dysfunction; incidence of tumors or persistent hypospermia.

The term “therapeutically effective amount” refers to the amount or dose of ANP polypeptide which provides the desired effect in the patient. In the case of ANP polypeptides with extended pharmacokinetic profiles, such a dose may be the amount given upon single or multiple dose administration. Determining an effective amount can be readily accomplished by persons of skill in the art through the use of known techniques and by observing results obtained under analogous circumstances.

With regard to a route of administration, the ANP polypeptides or pharmaceutical composition including the same can be administered in accord with known methods such as, for example, orally; by injection (i.e., intra-arterially, intravenously, intraperitoneally, intracerebrally, intracerebroventricularly, intramuscularly, intraocularly, intraportally or intralesionally), by sustained release systems, or by implantation devices. Administration of ANP polypeptides according to the present invention is typically parenteral, e.g., intravenous (IV), subcutaneous (SC or SQ) or intraperitoneal (IP). Thus, in certain embodiments of the present invention, ANP polypeptides are administered intravenously. In other embodiments of the present invention. ANP polypeptides are administered intraperitoneally. In other embodiments, ANP polypeptides are administered subcutaneously. In certain instances, the ANP polypeptides or pharmaceutical composition including the same can be administered SQ by bolus injection or continuously.

The present invention also encompasses novel intermediates and processes useful for the production of ANP polypeptides of the present invention. The intermediates and ANP polypeptides of the present invention may be prepared by a variety of procedures known in the art, including processes using chemical synthesis such as those described in the Examples below or biological expression.

With respect to chemical synthesis, one can use standard manual or automated solid-phase synthesis procedures. For example, automated peptide synthesizers are commercially available from, for example, CEM (Charlotte, North Carolina), CSBio (Menlo Park, California) and Gyros Protein Technologies Inc. (Tucson, AZ). Reagents for solid-phase synthesis are readily available from commercial sources. Solid-phase synthesizers can be used according to the manufacturer's instructions for blocking interfering groups, protecting amino acids during reaction, coupling, deprotecting and capping of unreacted amino acids.

With respect to biological expression, one can use standard recombinant techniques to construct a polynucleotide having a nucleic acid sequence that encodes an amino acid sequence for all or part of an ANP polypeptide, incorporate that polynucleotide into recombinant expression vectors, and introduce the vectors into host cells, such as bacteria, yeast and mammalian cells, to produce the ANP polypeptide. See, e.g., Green & Sambrook, “Molecular Cloning: A Laboratory Manual” (Cold Spring Harbor Laboratory Press, 4^{th} ed. 2012). The polypeptides may readily be produced in mammalian cells such as CHO, NSO, HEK293, BHK, or COS cells; in bacterial cells such as E. coli. Bacillus subtilis, or Pseudomonas fluorescens; in insect cells, or in fungal or yeast cells, which are cultured using techniques known in the art. The vectors containing the polynucleotide sequences of interest can be transferred into the host cell by well-known methods, which vary depending on the type of cellular host. Various methods of protein purification may be employed and such methods are known in the art.

As noted above, all HF patients, even those who are mildly symptomatic are at high risk of dying. Thus, when used herein, references to a “patient in need” of a treatment for heart failure (HF) may refer to a broad range of individuals having HF, including those with a broad range disease severity as described below. The New York Heart Association (NYHA) has provided a classification scheme for the degree or severity of HF, as summarized below.

| NYHA | |
|---|---|
| Class | Symptoms |
| I | No limitation of physical activity. Ordinary physical activity does not cause |
| | undue fatigue, palpitation, dyspnea (shortness of breath). |
| II | Slight limitation of physical activity. Comfortable at rest. Ordinary physical |
| | activity results in fatigue, palpitation, dyspnea (shortness of breath). |
| III | Marked limitation of physical activity. Comfortable at rest. Less than ordinary |
| | activity causes fatigue, palpitation, or dyspnea. |
| IV | Unable to carry on any physical activity without discomfort. Symptoms of |
| | heart failure at rest. If any physical activity is undertaken, discomfort |
| | increases. |

In certain embodiments, the patient in need is in heart failure NYHA Class II-IV. In certain embodiments, the patient in need is in heart failure NYHA Class II. In certain embodiments, the patient in need is in heart failure NYHA Class III. In certain embodiments, the patient in need is in heart failure NYHA Class IV. In certain embodiments, the patient in need is in heart failure NYHA Class II-III.

As noted above, existing therapeutic treatment options for heart failure, including current standard of care, improve symptoms and slow down disease progression through hemodynamic mechanisms—e.g., reducing blood pressure, heart rate and/or plasma volume—to reduce the workload of the failing heart. The ANP polypeptides of the present invention, by contrast, achieve their effects through a different mechanism of action, namely, selective NPR-A binding and the activity resulting therefrom to provide biomarker (cGMP. NT-proBNP), hemodynamic (LVEDP), structural (LA Volume), and symptomatic (lung congestion, dyspnea) improvements, thus improving outcomes and QoL for HFpEF patients. Due to these different mechanisms of action, ANP polypeptides of the present invention can be administered on top of existing SoC without titration or monitoring. Thus, in certain embodiments, ANP polypeptides of the present invention may be administered in combination with one or more additional treatments for heart failure. In certain embodiments, the one or more additional treatments for heart failure are selected from administration of therapeutic agents such as anticoagulants, beta blockers, ACE inhibitors, ARBs, ARNIs, MRAs, diuretics, digitalis, digoxin, hydralazine/isosorbide dinitrate, ivabradine, ARB and NEP inhibitor combination (sacubitril/valsartan (ENTRESTO®)), statins and/or anti-glycemic agents, as well as other therapeutic agents to control comorbidities, including, but not limited to, high cholesterol, high blood pressure, atrial fibrillation and diabetes. In certain embodiments, ANP polypeptides of the present invention may be administered in combination with SGLT2 inhibitors or sGC activators.

The additional therapeutic agent can be administered simultaneously, separately or sequentially with the ANP polypeptide or pharmaceutical composition including the same. Moreover, the additional therapeutic agent can be administered with a frequency same as the ANP polypeptide or pharmaceutical composition including the same (i.e., every other day, twice a week, or weekly). Alternatively, the additional therapeutic agent can be administered with a frequency distinct from the ANP polypeptide or pharmaceutical composition including the same. In other instances, the additional therapeutic agent can be administered SQ. In other instances, the additional therapeutic agent can be administered IV. In still other instances, the additional therapeutic agent can be administered orally.

It is further contemplated that the methods may be combined with diet and exercise and/or may be combined with additional therapeutic agents other than those discussed above.

The ANP polypeptides herein can be formulated as pharmaceutical compositions, which can be administered by parenteral routes (e.g., intravenous, intraperitoneal, intramuscular, subcutaneous or transdermal). Such pharmaceutical compositions and techniques for preparing the same are well known in the art. See, e.g., Remington, “The Science and Practice of Pharmacy” (D. B. Troy ed., 21st Ed., Lippincott, Williams & Wilkins, 2006). In particular instances, the ANP polypeptides are administered SQ or IV. Alternatively, however, the ANP polypeptides can be formulated in forms for other pharmaceutically acceptable routes such as, for example, tablets or other solids for oral administration; time release capsules, and any other form currently used, including creams, lotions, inhalants and the like.

As noted above, and to improve their in vivo compatibility and effectiveness, the ANP polypeptides herein may be reacted with any number of inorganic and organic acids/bases to form pharmaceutically acceptable acid/base addition salts. Pharmaceutically acceptable salts and common techniques for preparing them are well known in the art (see, e.g., Stahl et al., “Handbook of Pharmaceutical Salts: Properties, Selection and Use” (2nd Revised Ed. Wiley-VCH, 2011)). Pharmaceutically acceptable salts for use herein include sodium, trifluoroacetate, hydrochloride and acetate salts.

The ANP polypeptides herein may be administered by a physician or self-administered using an injection. It is understood the gauge size and amount of injection volume can be readily determined by one of skill in the art. However, the amount of injection volume can be ≤about 2 mL or even ≤about 1 mL, and the needle gauge can be ≥about 27 G or even ≥about 29 G.

The ANP polypeptides herein can also be provided as part of a kit. In some instances, the kit includes a device for administering at least one ANP polypeptide (and optionally at least one additional therapeutic agent) to an individual. In certain instances, the kit includes a syringe and needle for administering the at least one ANP polypeptide (and optionally at least one additional therapeutic agent). In particular instances, the ANP polypeptide (and optionally at least one additional therapeutic agent) is pre-formulated in aqueous solution within the syringe.

The invention is further illustrated by the following examples, which are not to be construed as limiting.

### EXAMPLES

### Preparations

Abbreviations: acetonitrile (ACN); aqueous (aq); octadecylsilane (C18); dichloromethane (DCM); N,N-dimethylformamide (DMF); dimethylsulfoxide (DMSO); ethyl acetate (EtOAc); hexafluorophosphate azabenzotriazole tetramethyl uranium (HATU); high performance liquid chromatography (HPLC); isopropanol (IPA); liquid chromatography mass spectrometry (LCMS); methanol (MeOH); minute(s) (min); mass spectrometry (MS); methyl tert-butyl ether (MTBE); mass-to-charge ratio (m/z); polyethylene glycol (PEG); reverse-phase high performance liquid chromatography (RP-HPLC); reverse-phase liquid chromatography mass spectrometry (RP-LCMS); room temperature (rt); saturated (satd); strong cation exchange (SCX); tris(2-carboxyethyl)phosphine (TCEP); trifluoroacetic acid (TFA); tetrahydrofuran (THF); tris(hydroxymethyl)aminomethane (Tris).

### Preparation 1

### Tert-butyl 11-bromoundecanoate

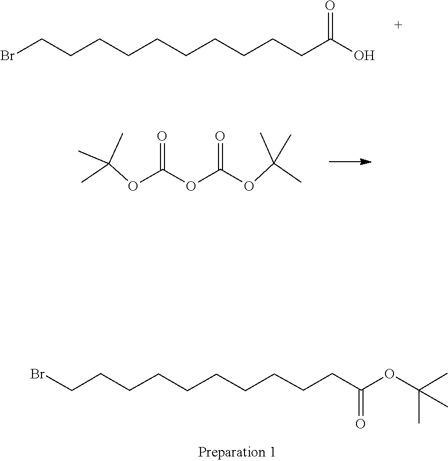 [SMILES: O=C(O)CCCCCCCCCCBr ; CC(C)(C)OC(=O)OC(=O)OC(C)(C)C ; CC(C)(C)OC(=O)CCCCCCCCCCBr]

Under a nitrogen atmosphere, a pressure vessel was charged with di-tert-butyl dicarbonate (8.65 g, 39.2 mmol) and a mixture of 11-bromoundecanoic acid (8.00 g, 30.2 mmol), dichloromagnesium hexahydrate (613 mg, 3.01 mmol) in tert-butanol (60 mmol). The vessel was sealed, and then heated to 40° C. for 24 hours. The solution was diluted with dichloromethane (100 mL) and washed with saturated ammonium chloride (3×50 mL). The organic phase was then dried over sodium sulfate, concentrated in vacuo to dryness, and purified by flash column chromatography (120 g silica column, gradient from 100% Hexane to 100% EtOAc in Hexane over 20 minutes). The desired product was isolated as a colorless oil (6.05 g); mz=265, 267 (M-tBu).

### Preparation 2

### O1-benzyl O3-tert-butyl 2-undecylpropanedioate

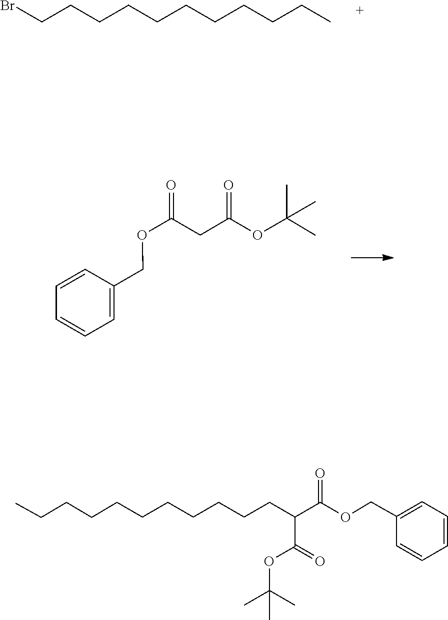

Under a nitrogen atmosphere, sodium hydride was added in mineral oil (60 mass %, 400 mg, 10.0 mmol) in small portions to an ice-cold solution of O1-benzyl O3-tert-butyl propanedioate (2.50 g, 9.99 mmol) in N,N-dimethylformamide (15 mL). After stirring for 1 hour, 1-bromoundecane (2.35 g, 9.99 mmol) was added in 2 mL of DMF and mixed at room temperature for 15 hours. The mixture was diluted with 60 mL of ether, and washed the organic layer with 1% aqueous citric acid (50 mL), brine and water. The organic layer was dried over sodium sulfate and the volatiles removed in vacuo and purified by flash column chromatography (80 g silica column, gradient from 100% Hexane to 40% EtOAc over 25 minutes). The desired product was isolated as an oil (3.50 g); mz=403 (M-1).

### Preparation 3

### O11-benzyl O1,O11-ditert-butyl docosane-1,11,11-tricarboxylate

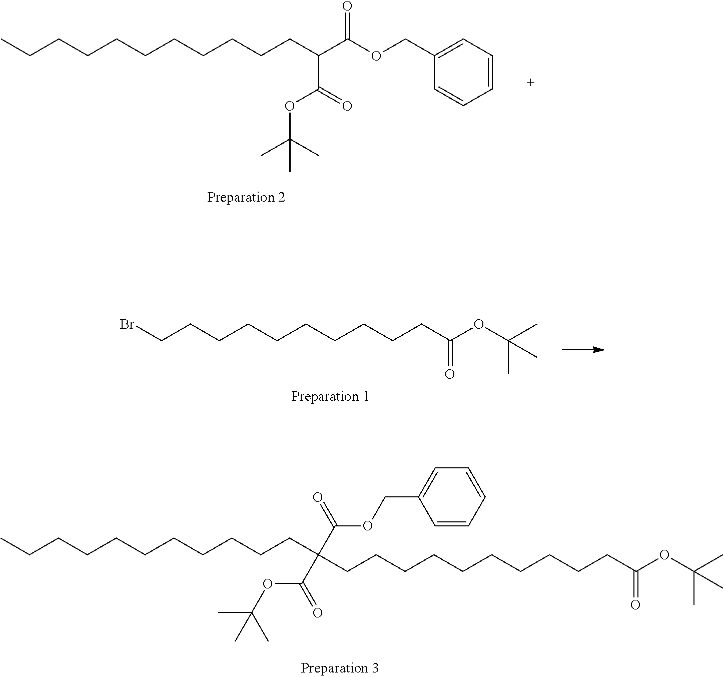 [SMILES: CCCCCCCCCCCC(C(=O)OCc1ccccc1)C(=O)OC(C)(C)C ; CC(C)(C)OC(=O)CCCCCCCCCCBr ; CCCCCCCCCCCC(CCCCCCCCCCC(=O)OC(C)(C)C)(C(=O)OCc1ccccc1)C(=O)OC(C)(C)C]

Under a nitrogen atmosphere, was added sodium hydride in mineral oil (60 mass %, 960 mg, 24.0 mmol) in small portions of an ice-cold mixture of O1-benzyl O3-tert-butyl 2-undecylpropanedioate (8.50 g, 20.0 mmol) in N,N-dimethylformamide (40 mL). Stirred at room temperature for 40 minutes. Added tert-butyl 11-bromoundecanoate (7.50 g, 22.2 mmol) in 10 mL of DMF. Allowed to mix at room temperature for 20 hours. Diluted the mixture with 150 mL of ether and washed the organic phase with 1% aqueous citric acid (50 mL), brine and water. Dried the organic layer over sodium sulfate and removed the volatiles in vacuo. Purified by flash column chromatography (220 g silica column, gradient from 100% Hexane to 100% DCM over 15 minutes, kept for another 10 minutes). The desired product was isolated as an oil (13.00 g); mz=533 (M-2×tBU).

### Preparation 4

### 13-tert-butoxy-2-tert-butoxycarbonyl-13-oxo-2-undecyl-tridecanoic Acid

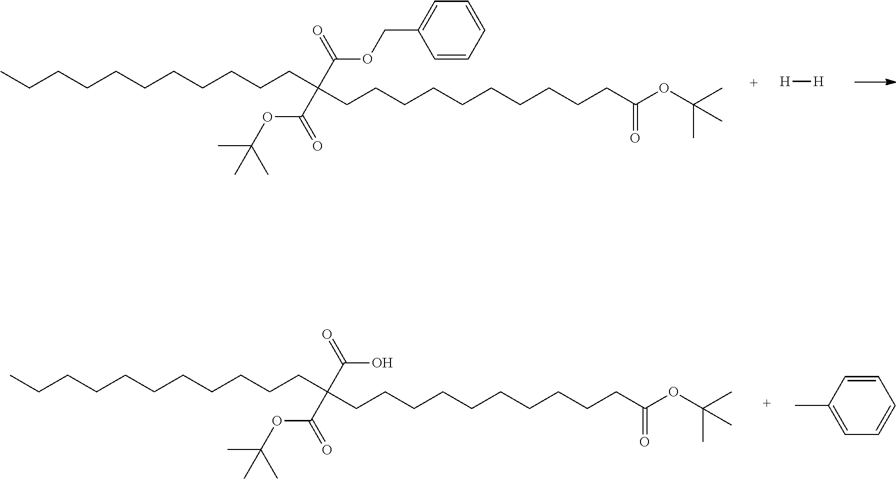

Charged a 2250 mL Parr shaker with 10% Pd/C (1.25 g), and purged with nitrogen. Added tetrahydrofran (125 mL) and then a solution of O11-benzyl O1,O11-ditert-butyl docosane-1,11,11-tricarboxylate (13.00 g, 19.15 mmol) in 125 mL of tetrahydrofuran. Sealed the bottle, purged with nitrogen, and pressurized with hydrogen gas at 10 psi. Shaken at room temperature for 2 hours. Depressurized the system with nitrogen gas, then filtered through Celite. Removed the solvent from the mixture under reduced pressure to isolate the racemic product as a white solid (11.0 g); mz=443 (M-2×t-Butyl).

### Preparation 5

### Benzyl 11-bromoundecanoate

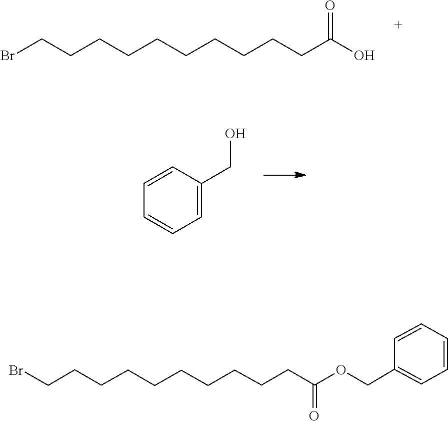 [SMILES: O=C(O)CCCCCCCCCCBr ; OCc1ccccc1 ; O=C(CCCCCCCCCCBr)OCc1ccccc1]

Dissolved 11-bromoundecanoic acid (10.00 g, 37.71 mmol), benzylalcohol (4.5 g, 42 mmol) and 4-dimethylaminopyridine (0.4 g, 3 mmol) in dichloromethane (150 mL). To the solution, added dicyclohexylcarbodiimide (9.40 g, 45.6 mmol, 100 mass %) in one portion. Stirred at room temperature for 8 hours. Removed the white solids by filtration and washed the solid with dichloromethane (3×10 mL). Removed the organic components under reduced pressure. Purified by flash column chromatography (220 g silica column, gradient from 100% Hexane to 100% dichloromethane over 20 minutes, continued for another 5 minutes). Combined the product containing fractions to isolate product as an oil (11.60 g); ¹H NMR (400 MHz, CDCl₃): 7.39-7.36 (m, 5H), 5.14 (s, 2H), 3.43 (t, J=6.9 Hz, 2H), 2.38 (t, J=7.6 Hz, 2H), 1.91-1.84 (m, 2H), 1.67 (quintet, J=7.3 Hz, 2H), 1.43 (dd, J=7.0, 14.4 Hz, 2H), 1.30 (s, 10H).

### Preparation 6

### O1-benzyl O3-tert-butyl 2-undecylpropanedioate

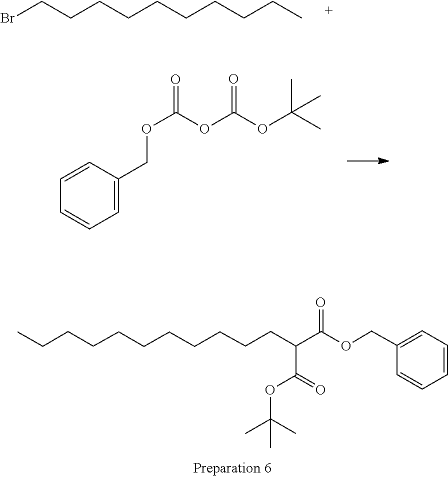

Under a nitrogen atmosphere, added sodium hydride in mineral oil (60 mass %, 400 mg, 10.0 mmol) in small portions to an ice-cold solution of O1-benzyl O3-tert-butyl propanedioate (2.50 g, 9.99 mmol) in N,N-dimethylformamide (15 mL), After stirring for 1 hour, added 1-bromoundecane (2.35 g, 9.99 mmol) in 2 mL of DMF. Mixed at room temperature for 15 hours. Diluted the mixture with 60 mL of ether and washed the organic layer with 1% aqueous citric acid (50 mL), brine and water. Dried the organic layer over sodium sulfate and removed the volatiles in vacuo. Purified by flash column chromatography (80 g silica column, gradient from 100% Hexane to 40% EtOAc over 25 minutes). The desired product was isolated as an oil (3.50 g); mz=403 (M-1).

### Preparation 7

### O1,O11-dibenzyl O11-tert-butyl docosane-1,11,11-tricarboxylate

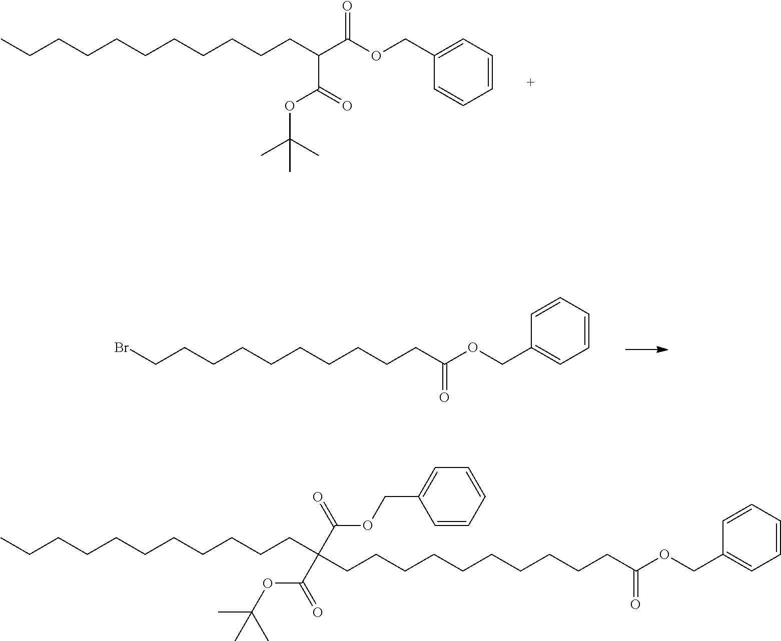 [SMILES: CCCCCCCCCCCC(C(=O)OCc1ccccc1)C(=O)OC(C)(C)C ; O=C(CCCCCCCCCCBr)OCc1ccccc1 ; CCCCCCCCCCCC(CCCCCCCCCCC(=O)OCc1ccccc1)(C(=O)OCc1ccccc1)C(=O)OC(C)(C)C]

Under a nitrogen atmosphere, added sodium hydride in mineral oil (60 mass %, 700 mg, 17.5 mmol) in small portions to an ice-cold solution of O1-benzyl O3-tert-butyl 2-undecylpropanedioate (6.2 g, 14.6 mmol) in N,N-dimethylformamide (30 mL). After 40 minutes, added benzyl 11-bromoundecanoate (6.00 g, 16.0 mmol) in 8 mL of DMF. Mixed at room temperature for 15 hours. Diluted the mixture with 150 mL of ether. Washed the mixture with citric acid (1%, in water, 50 mL), brine & water. Dried the organic layer over sodium sulfate and removed the volatiles in vacuo. Purified by flash column chromatography (220 g silica column, gradient from 100% Hexane to 100% DCM over 20 minutes, kept for another 10 minutes). The desired product was isolated as an oil (8.00 g); mz=624 (M-tBu), 702 (M+Na).

### Preparation 8

### 13-Benzyloxy-2-benzyloxycarbonyl-13-oxo-2-undecyl-tridecanoic Acid

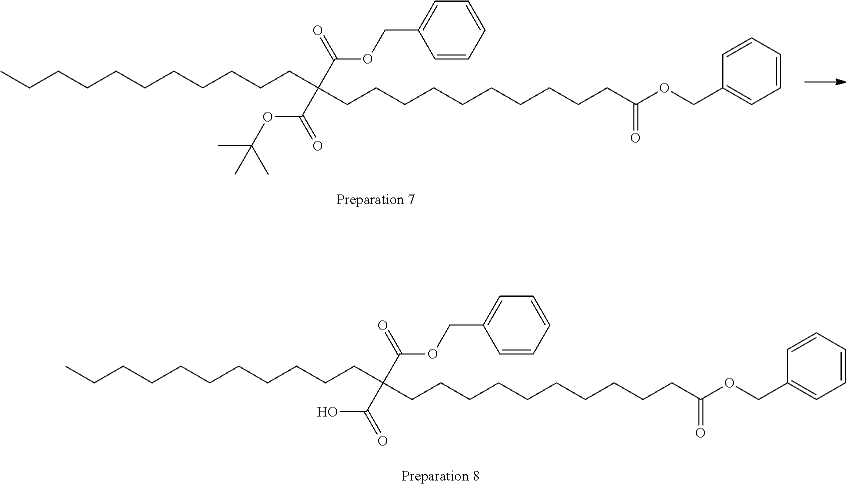 [SMILES: CCCCCCCCCCCC(CCCCCCCCCCC(=O)OCc1ccccc1)(C(=O)OCc1ccccc1)C(=O)OC(C)(C)C ; CCCCCCCCCCCC(CCCCCCCCCCC(=O)OCc1ccccc1)(C(=O)O)C(=O)OCc1ccccc1]

Treated O1,O11-dibenzyl O11-tert-butyl docosane-1,11,11-tricarboxylate (11.0 g, 15.4 mmol) with trifluoroacetic acid (40 mL) at room temperature for 3 hours. Removed the volatiles to a residue and purified by flash column chromatography (120 g silica column, gradient from 100% hexane to 100% EtOAc in Hexane over 20 minutes). The desired product was isolated as an oil (9.5 g); mz=623 (M+1); ¹H NMR (400 MHz, CDCl₃): 8.77-8.75 (m, 1H), 7.39-7.38 (m, 10H), 5.26 (s, 2H), 5.14 (s, 2H), 2.38 (t, J=7.5 Hz, 2H), 2.02-1.84 (m, 4H), 1.66 (quintet, J=7.4 Hz, 2H), 1.31-0.89 (m, 37H).

### Chiral Separation of Racemic Compound 13(benzyloxy)-2-((benzyloxy)carbonyl)-13-oxo-2-undecyltridecanoic Acid (Preparation 8) into Enantiomer 1 (Preparation 8A, EN1) and Enantiomer 2 (Preparation 8B, EN2)

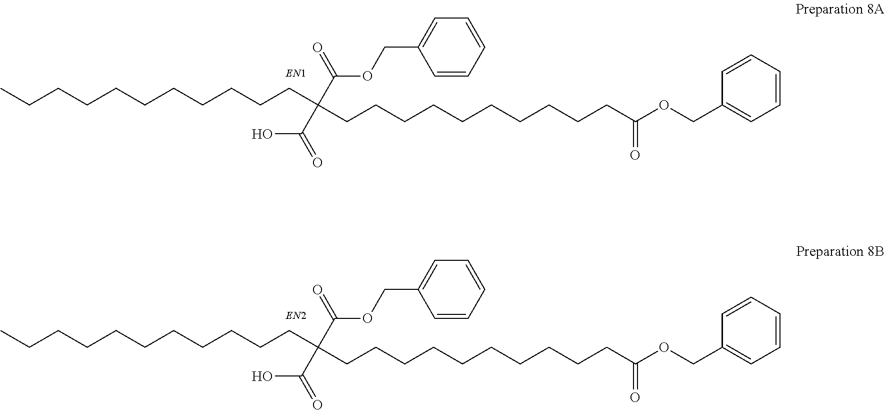 [SMILES: CCCCCCCCCCCC(CCCCCCCCCCC(=O)OCc1ccccc1)(C(=O)O)C(=O)OCc1ccccc1 ; CCCCCCCCCCCC(CCCCCCCCCCC(=O)OCc1ccccc1)(C(=O)O)C(=O)OCc1ccccc1]

### Preparative Method:

Column Used: Chiralpak AD-H, 21×150 mm
Mobile Phase: 20% EtOH: 80% CO₂
Flow Rate: 80 mL/min
BPR Set Point: 100 bar
BPR Temperature: 20° C.
Column Temperature: 40° C.
Detection: 225 nm

### Analytical Conditions:

Chiralpak AD-H, 4.6×150 mm, 25% EtOH/CO₂, 5 mL/min, 225 nm

From 1300 mg of racemic compound, using the conditions for preparative method, enantiomer 1 (EN1; 564 mg, 99% ee, retention time=2.64 min) and enantiomer 2 (EN2; 511.2 mg, 98% ee, retention time=3.21 min) were isolated.

### Preparation 9

### Beta-Ala Linker

### O1,O11-dibenzyl O11-(2,5-dioxopyrrolidin-1-yl) docosane-1,11,11-tricarboxylate

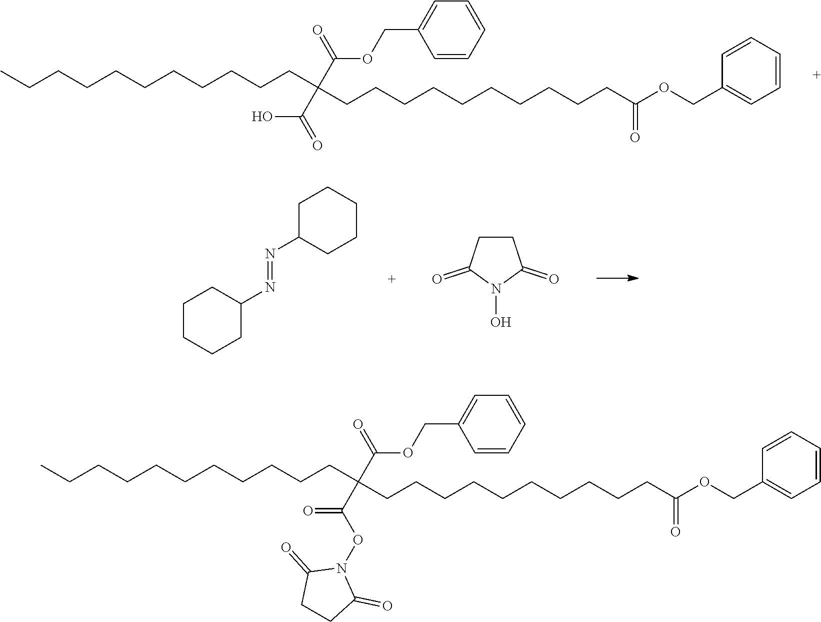

Added N-hydroxysuccinimide (0.500 g, 4.25 mmol) to 13-benzyloxy-2-benzyloxycarbonyl-13-oxo-2-undecyl-tridecanoic acid (2.45 g, 3.54 mmol) in dichloromethane (20 mL) and THF (5 mL). Stirred for five minutes, then added N,N′-dicyclohexylcarbodiimide (0.880 g, 4.22 mmol) in one portion. Stirred for 7 hours under nitrogen atmosphere at room temperature. Stored the reaction mixture in −20° C. fridge for two days. Removed the solid by filtration, and washed the solid with DCM (3×5 mL). Removed the solvent from the filtrate and purified by flash column chromatography (80 g silica column, gradient from 100% hexane to 50% EtOAc in hexane over 20 minutes, then increased to 100% EtOAc over 5 minutes). The desired product was isolated as an oil (2.10 g); ¹H NMR (400 MHz, CDCl₃): 7.43-7.34 (m, 10H), 5.25 (s, 2H), 5.14 (s, 2H), 4.15 (q, J=7.2 Hz, 1H), 2.84 (d, J=3.1 Hz, 4H), 2.37 (t, J=7.6 Hz, 2H), 2.02-1.97 (m, 4H), 1.69-1.59 (m, 3H), 1.34-1.25 (m, 38H), 0.90 (t, J=6.8 Hz, 3H).

### Preparation 10

### 3-[(13-Benzyloxy-2-benzyloxycarbonyl-13-oxo-2-undecyl-tridecanoyl)amino]propanoic Acid

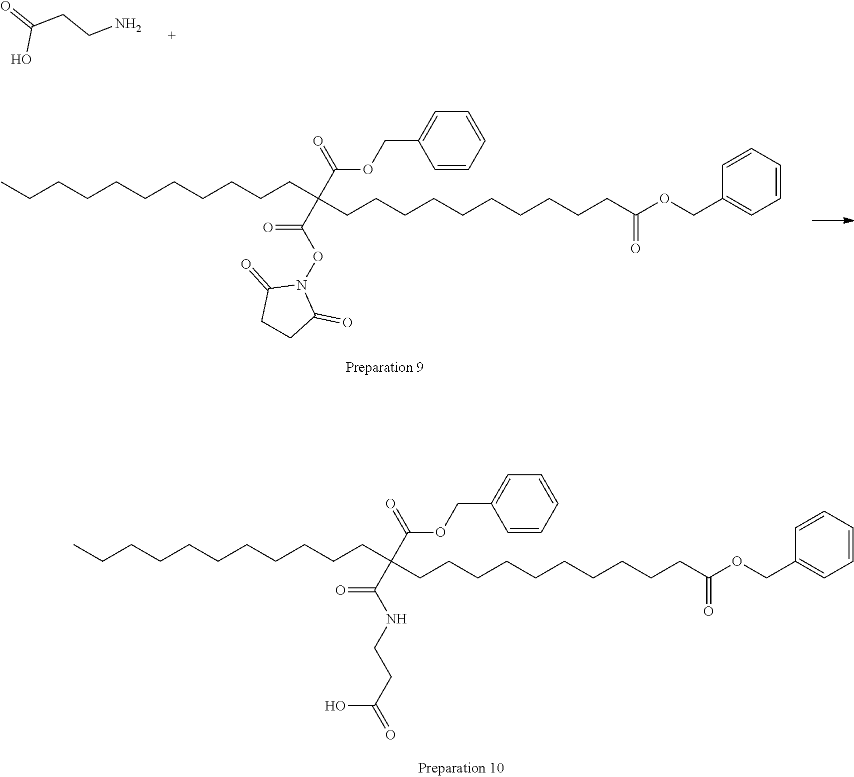 [SMILES: NCCC(=O)O ; CCCCCCCCCCCC(CCCCCCCCCCC(=O)OCc1ccccc1)(C(=O)OCc1ccccc1)C(=O)ON1C(=O)CCC1=O ; CCCCCCCCCCCC(CCCCCCCCCCC(=O)OCc1ccccc1)(C(=O)NCCC(=O)O)C(=O)OCc1ccccc1]

Added a suspension of beta-Alanine (250 mg, 2.80605 mmol) in 1 mL of DMF to a room temperature solution of O1,O11-dibenzyl O11-(2,5-dioxopyrrolidin-1-yl) docosane-1,11,11-tricarboxylate (1.50 g, 2.08 mmol) in tetrahydrofuran (20 mL), and followed by addition of triethylamine (0.80 mL, 5.7 mmol). Added water (3 mL), acetonitrile (6 mL, 100 mass %) and 4 mL DMF to solubilize the precipitate that forms. Mixed at room temperature for 15 hours. Diluted the mixture with chloroform/iso-propanol (3/1, 100 mL), and washed with 10% aqueous citric acid, water and brine (50 mL). Dried the organic over sodium sulfate, and concentrated in vacuo to dryness. Purified by flash column chromatography (80 g silica column, UV 254 nm, gradient from 100% hexane to 100% EtOAc over 15 minutes, kept for another 5 minutes). The desired product was isolated, 1.00 g, 66% yield) as an oil; mz=694 (M+).

### Preparation 11

### NHS Ester of Beta-Alanine BFA

### Dibenzyl 2-[[3-(2,5-dioxopyrrolidin-1-yl)oxy-3-oxo-propyl]carbamoyl]-2-undecyl-tridecanedioate

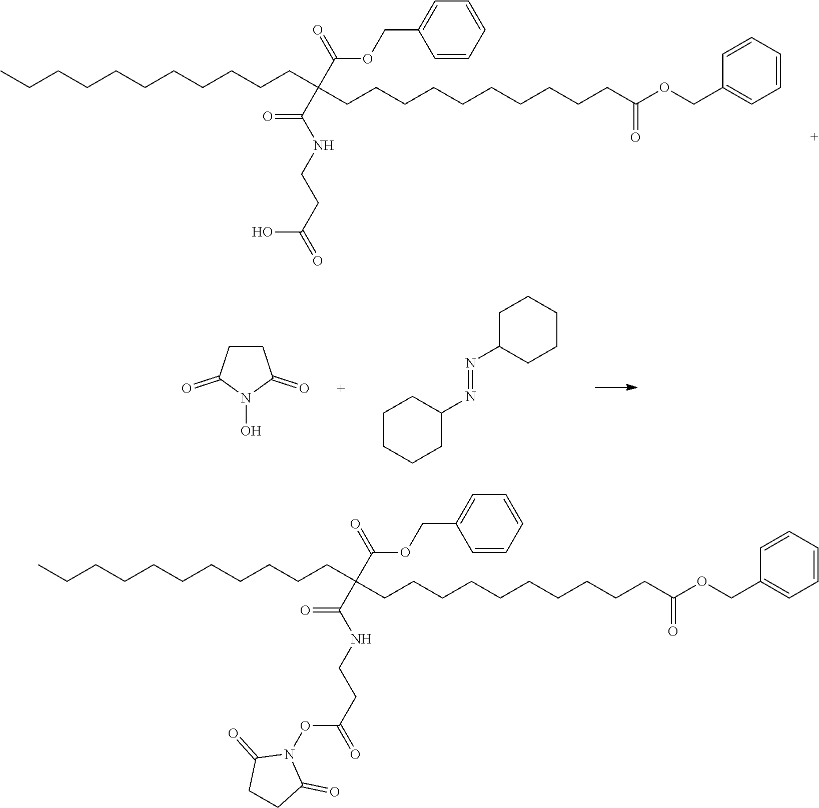 [SMILES: CCCCCCCCCCCC(CCCCCCCCCCC(=O)OCc1ccccc1)(C(=O)NCCC(=O)O)C(=O)OCc1ccccc1 ; O=C1CCC(=O)N1O ; C1CCC(/N=N/C2CCCCC2)CC1 ; CCCCCCCCCCCC(CCCCCCCCCCC(=O)OCc1ccccc1)(C(=O)NCCC(=O)ON1C(=O)CCC1=O)C(=O)OCc1ccccc1]

Added N-hydroxysuccinimide (193 mg, 1.64 mmol) to a solution of 3-[(13-benzyloxy-2-benzyloxycarbonyl-13-oxo-2-undecyl-tridecanoyl)amino]propanoic acid (1.00 g, 1.37 mmol) in dichloromethane (15 mL) and THF (2 mL). After 5 minutes, added N,N′-dicyclohexylcarbodiimide (342 mg, 1.64 mmol) in one portion. Stirred at room temperature for 15 hours. Removed the solid by filtration and washed the solid with DCM (3×5 mL). Concentrated under vacuo to dryness and purified by flash column chromatography (80 g silica column, gradient from 100% Hexane to 100% EtOAc in none over 20 minutes). The desired product was isolated as an oil (1.00 g); mz=793 (M+2); ¹H NMR (400 MHz, CDCl₃): 8.27 (t, J=6.0 Hz, 1H), 7.38-7.35 (m, 10H), 5.19 (s, 2H), 5.13 (s, 2H), 3.68 (q, J=6.2 Hz, 2H), 2.89-2.84 (m, 6H), 2.36 (t, J=7.6 Hz, 2H), 2.02-1.94 (m, 2H), 1.82-1.75 (m, 2H), 1.65 (quintet, J=7.4 Hz, 2H), 1.32-1.00 (m, 34H).

### Preparation 12

### Deprotection of Benzyl on Beta-Alanine-BFA

### 2-[[3-(2,5-Dioxopyrrolidin-1-yl)oxy-3-oxo-propyl]carbamoyl]-2-undecyl-tridecanedioic Acid

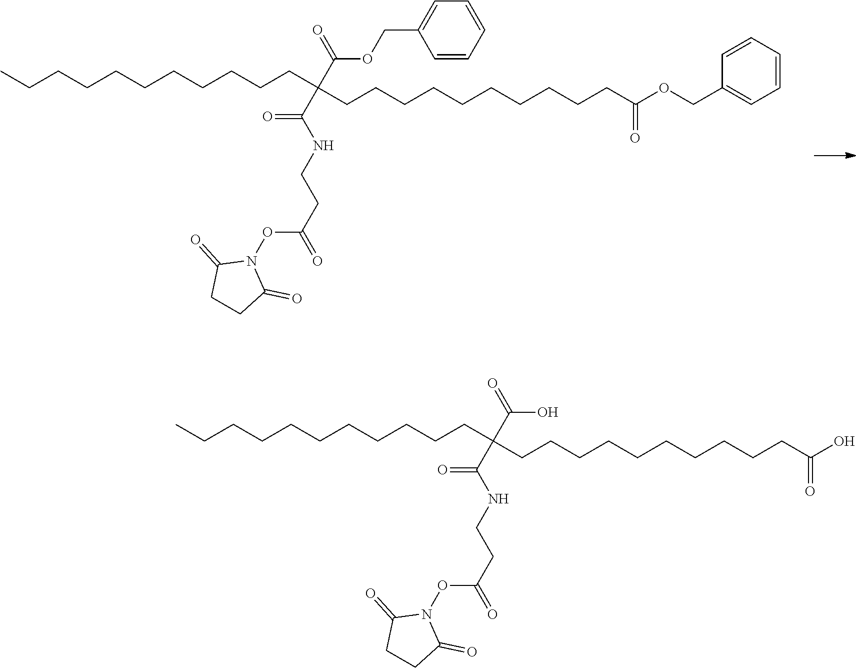 [SMILES: CCCCCCCCCCCC(CCCCCCCCCCC(=O)OCc1ccccc1)(C(=O)NCCC(=O)ON1C(=O)CCC1=O)C(=O)OCc1ccccc1 ; CCCCCCCCCCCC(CCCCCCCCCCC(=O)O)(C(=O)O)C(=O)NCCC(=O)ON1C(=O)CCC1=O]

Charged a 100 mL Parr shaker with 10% Pd/C (0.193 g), and purged with nitrogen. Added tetrahydrofuran (20 mL) and then a solution of dibenzyl 2-[[3-(2,5-dioxopyrrolidin-1-yl)oxy-3-oxo-propyl]carbamoyl]-2-undecyl-tridecanedioate (1.93 g, 2.68 mmol) in 20 mL of tetrahydrofuran. Sealed the bottle, purged with nitrogen, and pressurized with hydrogen gas at 10 psi. Shaken at room temperature for 2 hours. Depressurized the system with nitrogen gas, then filter through Celite. Removed the solvent from the mixture under reduced pressure to isolate the product as a solid (670 mg); mz=611 (M+).

### Preparation 13

### Alternative Chemistry for Amide Formation on Quaternary Acid

### Beta-Ala EN2

### 3-[[Ire-(2R)-13-benzyloxy-2-benzyloxycarbonyl-13-oxo-2-undecyl-tridecanoyl]amino]propanoic Acid

### Activated Ester

### O1,O11-dibenzyl O11-(triazolo[4,5-b]pyridin-3-yl) rel-(11S)-docosane-1,11,11-tricarboxylate

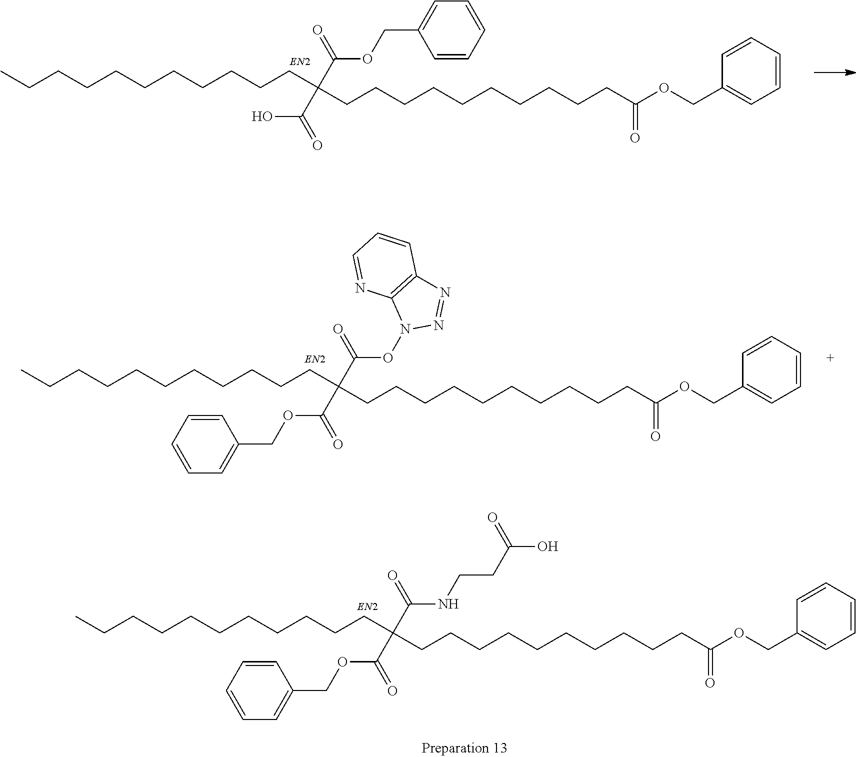 [SMILES: CCCCCCCCCCCC(CCCCCCCCCCC(=O)OCc1ccccc1)(C(=O)O)C(=O)OCc1ccccc1 ; CCCCCCCCCCCC(CCCCCCCCCCC(=O)OCc1ccccc1)(C(=O)OCc1ccccc1)C(=O)On1nnc2cccnc21 ; CCCCCCCCCCCC(CCCCCCCCCCC(=O)OCc1ccccc1)(C(=O)NCCC(=O)O)C(=O)OCc1ccccc1]

Under a nitrogen atmosphere, added [dimethylamino(triazolo[4,5-b]pyridin-3-yloxy)methylene]-dimethyl-ammonium; hexafluorophosphate (2.40 g, 6.12 mmol) to a solution of rel-(EN2)-13-benzyloxy-2-benzyloxycarbonyl-13-oxo-2-undecyl-tridecanoic acid (2.50 g, 4.01 mmol) in THF (10 mL, 100 mass %) and DMF (10 mL). Mixed at room temperature for 3 minutes, then cooled on an ice-bath prior to adding N,N-diisopropylethylamine (1.50 mL, 8.60 mmol). Stirred at room temperature for 3 hours. Diluted with DCM (60 mL), and washed with aqueous saturated solution of ammonium chloride (2×30 mL). Separated the organic layer, and dried over sodium sulfate. Purified the crude by normal phase flash chromatography (80 g silica gold column, 100% Hexane for 3 minutes, then gradient to 60% EtOAc in Hexane over 17 minutes, then switched to 100% EtOAc and keep for another 5 minutes). Isolated activated ester product and use directly onto next step.

### Coupling:

Mixed beta-Alanine (1.10 g, 12.3 mmol) and N,N-diisopropylethylamine (1.40 mL, 8.03 mmol) in 10 mL of acetonitrile and 9 mL of water. Dissolved the above activated ester in acetonitrile (10 mL) and then added into the beta alanine solution dropwise via syringe over 2 minutes. Stirred at room temperature for 1 hour. Diluted the reaction mixture with DCM (100 mL), and washed with saturated aqueous ammonium chloride (2×30 mL). Separated the organic layer, and dried over sodium sulfate. Concentrated under reduced pressure to a residue that was used as is in the next step (2.60 g).

### Preparation 14

### EN2-Beta-Ala-BFA by Activation/Deprotection

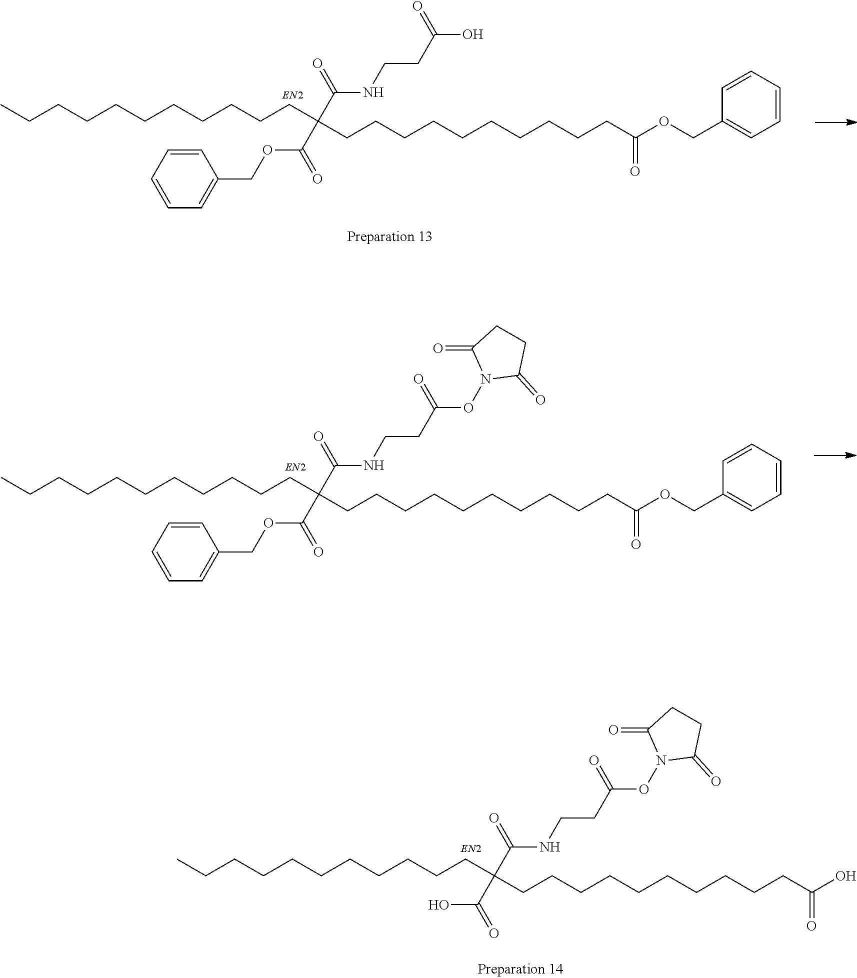 [SMILES: CCCCCCCCCCCC(CCCCCCCCCCC(=O)OCc1ccccc1)(C(=O)NCCC(=O)O)C(=O)OCc1ccccc1 ; CCCCCCCCCCCC(CCCCCCCCCCC(=O)OCc1ccccc1)(C(=O)NCCC(=O)ON1C(=O)CCC1=O)C(=O)OCc1ccccc1 ; CCCCCCCCCCCC(CCCCCCCCCCC(=O)O)(C(=O)O)C(=O)NCCC(=O)ON1C(=O)CCC1=O]

### Activation by NHS Ester

### Dibenzyl rei-(2R)-2-[[3-(2,5-dioxopyrrolidin-1-yl)oxy-3-oxo-propyl]carbamoyl]-2-undecyl-tridecanedioate

Added N-hydroxysuccinimide (240 mg, 2.04 mmol) to a mixture of 3-[[rel-(2R)-13-benzyloxy-2-benzyloxycarbonyl-13-oxo-2-undecyl-tridecanoyl]amino]propanoic acid (1.06 g, 1.53 mmol) in THF (5 mL) and dichloromethane (5 mL) at room temperature. Stirred for 3 minutes, then added N,N′-dicyclohexylcarbodiimide (420 mg, 2.01 mmol) in one portion followed by 1 mg of DMAP. Mixed at room temperature for 3 hours, and then stored in fridge for 12 hours. Removed the solid by filtration, and washed the solid with DCM (3×5 mL). Concentrated under reduced pressure, and purified by flash column chromatography (40 g silica/gold column, 100% Hexane for 5 minutes, gradient to 100% EtOAc over next 15 minutes). Purified a second time by flash chromatography (40 g column, 100% hexane 3 minutes, gradient to 100% MTBE over next 17 minutes). The desired product was isolated as a thick oil (1.0 g).

### Deprotection

Charged a 100 mL Parr shaker with 10% Pd/C (0.152 mg), and purged with nitrogen. Added tetrahydrofuran (10 mL) and then a solution of dibenzyl rel-(2R)-2-[[3-(2,5-dioxopyrrolidin-1-yl)oxy-3-oxo-propyl]carbamoyl]-2-undecyl-tridecanedioate (1.0 g, 1.20 mmol) in 10 mL of tetrahydrofuran. Sealed the bottle, purged with nitrogen, and pressurized with hydrogen gas at 10 psi. Shaken at room temperature for 2 hours. Depressurized the system with nitrogen gas, then filtered through Celite. Removed the solvent from the mixture under reduced pressure to isolate the product as a solid (750 mg); mz=611 (M+).

### Preparation 15

### Gamma-Glu-EN2-Synthesis

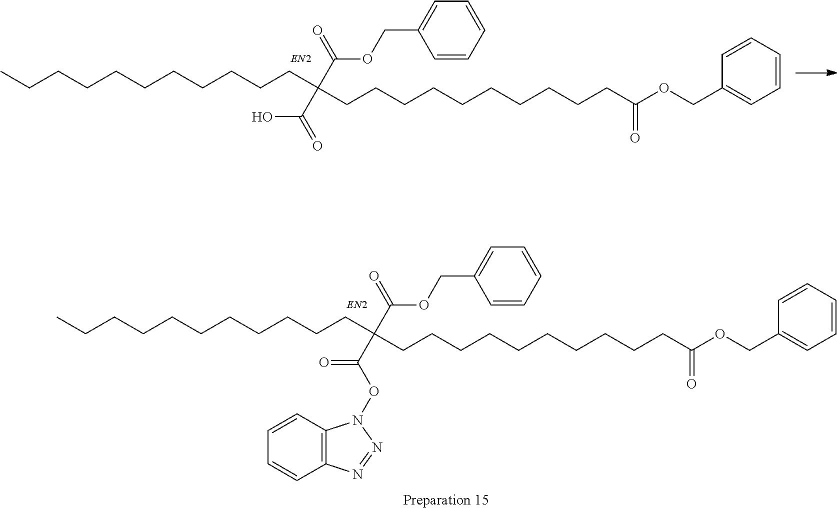 [SMILES: CCCCCCCCCCCC(CCCCCCCCCCC(=O)OCc1ccccc1)(C(=O)O)C(=O)OCc1ccccc1 ; CCCCCCCCCCCC(CCCCCCCCCCC(=O)OCc1ccccc1)(C(=O)OCc1ccccc1)C(=O)On1nnc2ccccc21]

### HBTU Ester

### O11-(benzotriazol-1-yl) O1,O11-dibenzyl rel-(11R)-docosane-1,11,11-tricarboxylate

Under a nitrogen atmosphere, mixed EN2-(2S)-13-benzyloxy-2-benzyloxycarbonyl-13-oxo-2-undecyl-tridecanoic acid (1.40 g, 2.25 mmol) and added [dimethylamino(triazolo[4,5-b]pyridin-3-yloxy)methylene]-dimethyl-ammonium; hexafluorophosphate (1.43 g, 3.65 mmol) in DMF (5 mL) and THF (5 mL). Cooled down to 10° C. on an ice-water bath, and then added N,N-diisopropylethylamine (0.85 mL, 4.9 mmol). Stirred at room temperature for 3 hours. Diluted the mixture with DCM (60 mL), and washed with saturated ammonium chloride (2×30 mL). Separated the organic layer, dried over sodium sulfate and concentrated in vacuo to dryness. Purified by normal phase flash chromatography (80 g silica gold column, 100% hexane for 3 minutes, then gradient to 60% EtOAc in hexane over 17 minutes, then switched to 100% EtOAc & kept for another 5 minutes). The desired product was isolated as an oil (1.15 g); ¹H NMR (400 MHz, CDCl₃): 8.68 (dd, J=1.3, 4.5 Hz, 1H), 8.40 (dd, J=1.4, 8.4 Hz, 1H), 7.52-7.50 (m, 2H), 7.43-7.36 (m, 10H), 5.38 (s, 2H), 5.14 (s, 2H), 2.38 (t, J=7.5 Hz, 2H), 2.18 (t, J=7.8 Hz, 4H), 1.68 (quintet, J=7.2 Hz, 2H), 1.36-1.30 (m, 35H), 0.91 (t, J=6.8 Hz, 3H).

### Preparation 16

### Gamma-Glu Coupling onto EN2-Activated Ester

### (4S)-4-[[(2S*)-13-benzyloxy-2-benzyloxycarbonyl-13-oxo-2-undecyl-tridecanoyl]amino]-5-tert-butoxy-5-oxo-pentanoic Acid

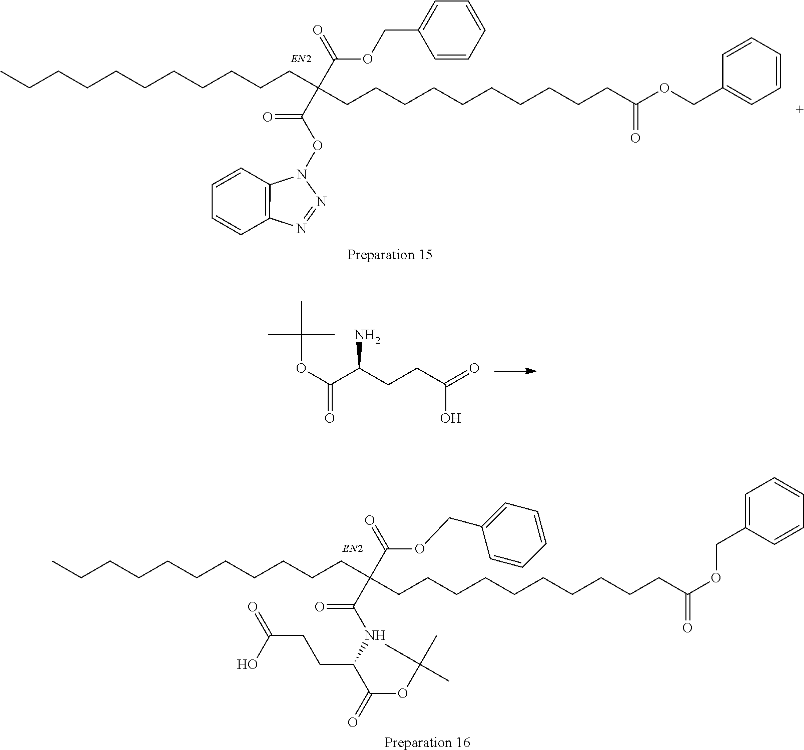 [SMILES: CCCCCCCCCCCC(CCCCCCCCCCC(=O)OCc1ccccc1)(C(=O)OCc1ccccc1)C(=O)On1nnc2ccccc21 ; CC(C)(C)OC(=O)[C@@H](N)CCC(=O)O ; CCCCCCCCCCCC(CCCCCCCCCCC(=O)OCc1ccccc1)(C(=O)OCc1ccccc1)C(=O)N1[C@@H](CCC(=O)O)C(=O)OC1(C)C ; "Preparation 16"]

Added N,N-diisopropylethylamine (0.55 mL, 3.2 mmol) to a solution of (4S)-4-amino-5-tert-butoxy-5-oxo-pentanoic acid (650 mg, 3.13 mmol) dissolved in acetonitrile (4 mL) and water (4 mL). Then added O11-(benzotriazol-1-yl) O1,O11-dibenzyl rel-(11R)-docosane-1,11,11-tricarboxylate (1.15 g, 1.55 mmol) in acetonitrile (3 mL). Stirred at room temperature for 12 hours. Diluted with 50 mL of DCM, and washed with 50 mL of aqueous ammonium chloride (2×). Separated the organic phase and dried over sodium sulfate. Concentrated in vacuo to dryness. Purified by normal phase flash chromatography (40 g silica gold column, 100% Hexane for 5 minutes, then gradient to 100% EtOAc in hexane over 15 minutes, kept for another 5 minutes). The desired product was isolated as an oil (900 mg); mz=806 (M-2); ¹H NMR (400 MHz, CDCl₃): 8.57 (d, J=7.5 Hz, 1H), 7.37-7.35 (m, 10H), 5.21 (s, 2H), 5.13 (s, 2H), 4.56 (td, J=7.7, 5.2 Hz, 1H), 4.14 (q, J=7.2 Hz, 1H), 2.44-2.35 (m, 4H), 2.27-2.20 (m, 1H), 2.02-1.93 (m, 3H), 1.85-1.77 (m, 2H), 1.65 (quintet, J=7.3 Hz, 2H), 1.32-1.18 (m, 34H), 0.90 (t, J=6.9 Hz, 3H).

### Preparation 17

### Two Step Procedure for Gamma-Glu-EN2-BFA

### (2S*)-2-[[(1S)-1-tert-butoxycarbonyl-4-(2,5-dioxopyrrolidin-1-yl)oxy-4-oxo-butyl]carbamoyl]-2-undecyl-tridecanedioic Acid

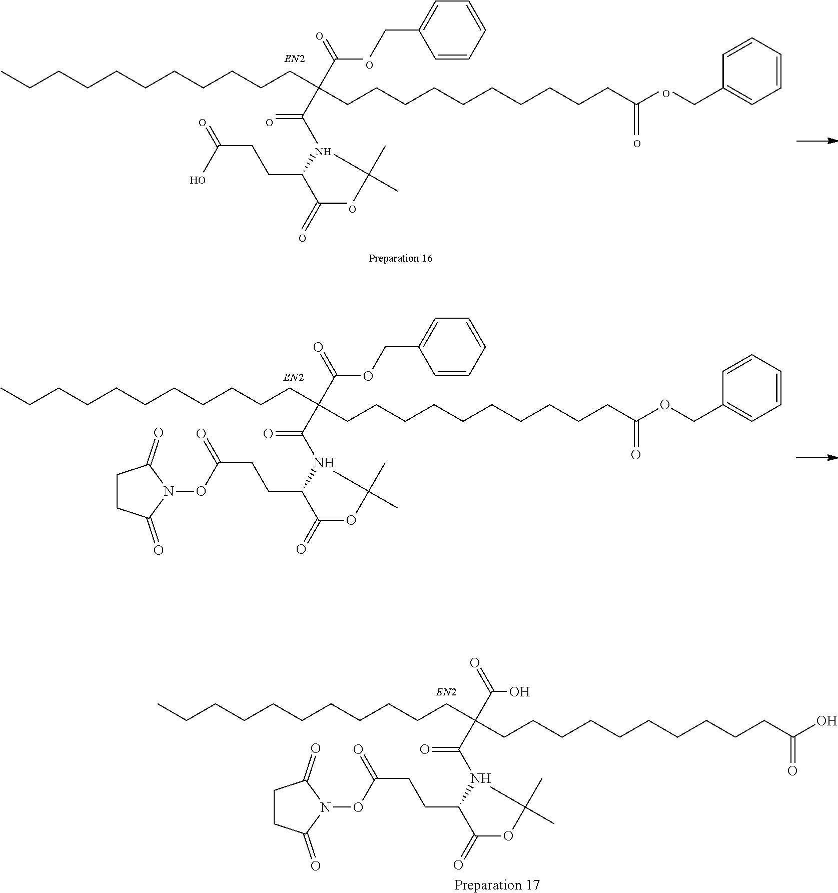 [SMILES: CCCCCCCCCCCC(CCCCCCCCCCC(=O)OCc1ccccc1)(C(=O)OCc1ccccc1)C(=O)N1[C@@H](CCC(=O)O)C(=O)OC1(C)C ; "Preparation 16" ; CCCCCCCCCCCC(CCCCCCCCCCC(=O)OCc1ccccc1)(C(=O)OCc1ccccc1)C(=O)N1[C@@H](CCC(=O)ON2C(=O)CCC2=O)C(=O)OC1(C)C ; CCCCCCCCCCCC(CCCCCCCCCCC(=O)O)(C(=O)O)C(=O)N1[C@@H](CCC(=O)ON2C(=O)CCC2=O)C(=O)OC1(C)C ; "Preparation 17"]

### First step: Activated ester

### Dibenzyl (2S*)-2-[[(1S)-1-tert-butoxycarbonyl-4-(2,5-dioxopyrrolidin-1-yl)oxy-4-oxo-butyl]carbamoyl]-2-undecyl-tridecanedioate

Under a nitrogen atmosphere, added N-hydroxysuccinimide (170 mg, 1.44 mmol) to a solution of (4S)-4-[[(2S*)-13-benzyloxy-2-benzyloxycarbonyl-13-oxo-2-undecyl-tridecanoyl]amino]-5-tert-butoxy-5-oxo-pentanoic acid (900 mg, 1.11 mmol,) in tetrahydrofuran (3 mL) and dichloromethane (6 mL). Mixed at room temperature for 3 minutes, then added N,N′-dicyclohexylcarbodiimide (300 mg, 1.43 mmol) as a solid and 1 mg of DMAP. Stirred at room temperature for 3 hours. Removed the white precipitate by filtration, and washed with DCM (3×5 mL). Concentrated in vacuo to dryness to provide crude activated ester. Purified by flash column chromatography (40 g silica column, 100% hexane for 5 minutes, gradient to 100% EtOAc over 15 minutes, kept for another 5 minutes). The activated ester was isolated (850 mg); mz=905 (M+).

### Second Step: Hydrogenation

Charged a 250 mL Parr shaker with 10% Pd/C (0.150 mg), and purged with nitrogen. Added tetrahydrofuran (10 mL) and then a solution of dibenzyl (2S*)-2-[[(1S)-1-tert-butoxycarbonyl-4-(2,5-dioxopyrrolidin-1-yl)oxy-4-oxo-butyl]carbamoyl]-2-undecyl-tridecanedioate (0.800 g, 0.883 mmol) in 15 mL of tetrahydrofuran. Sealed the bottle, purged with nitrogen, and pressurized with hydrogen gas at 20 psi. Shaken at room temperature for 2 hours. Depressurized the system with Nitrogen gas, then filtered through Celite. Removed the solvent from the mixture under reduced pressure to isolate the product as a solid (770 mg); mz=725 (M+1).

### Preparation of Example Polypeptides

### Example 1

Example 1 is a polypeptide represented by the following description (SEQ ID NO:45). HOOC—(CH₂)₁₈—CO-(γGlu)-EKEKEKGS-4Pal-RRSS[CFGGRIDRIGHQSGLGC]PSFRHGGPSSGAPPPS-NH₂

### (Disulfide Linkage)

Below is a depiction of the structure of Example 1 using the standard single letter code for L-Amino Acids except for the γ-Glutamic and 4-Pal residues, where the structures of the residues have been expanded.

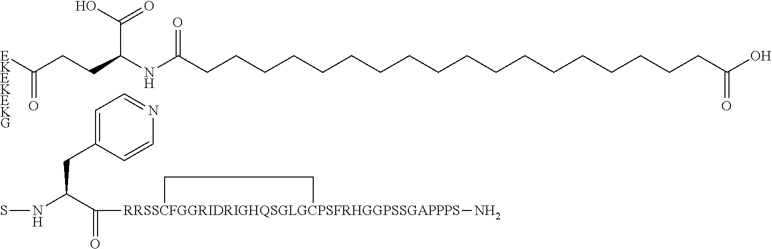

The primary peptide sequence of Example 1 was synthesized using standard 9-Fluorenyl-methyloxycarbonyl (Fmoc) tert-Butyl (t-Bu) solid phase peptide chemistry protocols on a Symphony-X, 24-channel multiplex peptide synthesizer (Gyros Protein Technologies, Inc.), at a 0.1 mmol scale.

The solid support used consists of low loading 4-(2′,4′-Dimethoxyphenyl-Fmoc-aminomethyl)-phenoxyacetamido-norleucyl-4-Methylbenzhydrylamine resin (Fmoc-Rink-MBHA Low Loading Resin, EMD Millipore), (100-200 mesh) with a 1% DVB cross-linked polystyrene core and a substitution range of 0.3-0.4 meq/g. Standard sidechain protecting groups were used for all Fmoc-L-Amino Acids used. The non-standard amino acids used in the synthesis of Example 1 were N-α-Fmoc-L-Glutamic Acid α-tButyl Ester (Fmoc-Glu-OtBu. Ark Pharm, Inc) and N-Fmoc-3-(4-Pyridyl)-L-Alanine (Fmoc-4Pal-OH, Combi-Blocks Inc.). Fmoc deprotection prior to each coupling step was accomplished by treatment with 20% piperidine (PIP: Sigma Aldrich) in dimethylformamide (DMF; Fisher Chemicals), 2×7 minutes with nitrogen mixing, followed by 8×DMF washing cycles. All amino acid couplings were performed for 1 hour using the Fmoc Amino Acid (0.3 M in DMF), N, N, N′,N′-Tetramethyl-O-(1H-benzotriazol-1-yl)uronium hexafluorophosphate (HBTU, Ambeed Inc.; 0.9 M in DMF) and N,N-Diisopropylethylamine (DIPEA, Sigma Aldrich; 1.2 M in DMF), at a 9-fold molar excess of AA/HBTU and a 12-fold molar excess of DIPEA over the theoretical resin loading level. After the primary sequence of the peptide was synthesized, the final Fmoc-deprotection, and the DMF washes were completed, attachment of the fatty acid (FA) moiety was accomplished by manual addition of 3-fold excess of 20-tert-butoxy-20-oxo-icosanoic acid (OtBu-C20-OH) solution which was pre-activated (2 min) with O-(7-Azabenzotriazol-1-yl)-N,N,N′,N′-tetramethyluronium hexafluorophosphate (HATU; Alfa Aesar) and DIPEA (1:1:3; FA:HATU:DIPEA) in 3 mL DMF. The solution was added via transfer pipet directly to the Symphony-X reaction vessel containing the peptidyl-resin. The reaction time for the FA coupling was 3 hours, after which point the resin was washed 3× with DMF and a Kaiser test was performed to ensure coupling completion. The FA coupling process is repeated as necessary if a positive Kaiser test in noted. After the FA acylation was completed, the peptidyl resin was transferred, as a DCM slurry, to disposable fritted plastic syringe fitted with Teflon stopcock and further washes with DCM were done, finally the resin was thoroughly dried in vacuo. The dry resin was then treated with 10 mL of cleavage cocktail consisting of trifluoroacetic acid (TFA), water, 3,6-dioxa-1,8-octanedithiol (DODT), triisopropylsilane (TIPS), (TFA:Water:DODT:TIPS; 92.5:2.5:2.5:2.5 v/v) for 2 h at RT. After the 2 hr incubation, the resin was filtered off, washed twice with 2 mL of neat TFA, and the combined filtrates/washes were collected in a 50 ml conical disposable tube, the solution was then treated with 35 mL of cold diethyl ether (−20° C.) to precipitate the crude peptide. The peptide/ether suspension was then centrifuged at 4000 rpm for 2 min to form a solid pellet, the supernatant was decanted, and the solid pellet was triturated with fresh ether and the process was repeated two additional times, finally drying the peptide pellet in vacuo.

### Disulfide Linkage Formation

The crude peptide was solubilized, in a suitable glass vessel, with 25% aqueous acetic acid to relatively low concentration (0.2-0.5 mg/ml crude peptide). The solution was then placed on magnetic stirrer with the requisite spin vane, mixed vigorously and titrated with a few drops of saturated Iodine in methanol solution until a faint yellow endpoint was achieved. After reaching the endpoint, the reaction was incubated at RT for 15 min, at which point the excess Iodine was quenched by the addition of a few drops of 0.1 M aqueous ascorbic acid.

### HPLC Purification

The crude oxidation solution was loaded directly onto a preparative HPLC system (Waters 2545 Binary Systems) equipped with a column heater and using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/Acetonitrile (ACN). The initial loading was done at 20% B, with 5 min isocratic wash after loading, then set to 25% B for equilibration. The sample was eluted using a linear 25-45% B gradient over 60 min, at a flow of 15 mL/min, with column heating set at 60° C. Fractions that were determined to contain the desired product (analysis by LC-MS) were pooled, frozen and lyophilized to give an amorphous solid product, as the TFA salt of Example 1. The purity assessed by RP-HPLC was found to be >95%, with the observed molecular weight of 5293.4 Dalton; matching the theoretical calculated molecular weight of 5293.9 Dalton.

### Example 2

Example 2 is a polypeptide represented by the following description (SEQ ID NO:60) HOOC—(CH₂)₁₈—CO-(γGlu)-EKEKEKGS-4Pal-RRSS[CFGGRIDRIGHQSGLGC]PSFRHGGPSSGAPPPS-NH2

### (Thioacetal Linkage)

Below is a depiction of the structure of Example 2 using the standard single letter code for L-Amino Acids except for the γ-Glutamic and 4Pal residues, and Cysteine residues where the structures of the residues have been expanded.

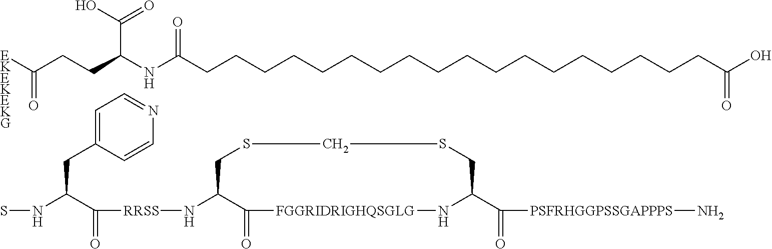

The primary peptide sequence of Example 2 is the same as Example 1. The disulfide linkage in Example 1 was replaced by a thioacetal linkage in Example 2. The synthesis of the acylated polypeptide of Example 2, formation of the disulfide linkage and purification were carried out as in Example 1.

### Thioacetal Linkage Formation of Example 2

After the purification of the disulfide bridged polypeptide form (same as Example 1), the pertinent pooled fractions containing the peptide were not lyophilized, but diluted with water and ACN instead to achieve about a 50/50 mixture of water/ACN (˜400 mL total volume) with a low concentration of peptide (˜0.2 mg/ml). The solution was then adjusted to pH 8 with triethylamine (TEA) ˜10 equivalents, and the peptide's disulfide bridge was reduced with the addition of 2-4 equivalents of Tris(2-carboxyethyl) phosphine hydrochloride (TCEP-HCl) reducing agent. After the disulfide bridge reduction, the thioacetal linkage was formed by the addition of 7-10 equivalents of diiodomethane (CH₂I₂). The thioacetal formation reaction was carried out by incubating the solution for 18 h at RT with magnetic stirring. Progress of the reaction was monitored using analytical LC-MS and by observing the change in mass of +12 Daltons from the starting reduced peptide molecular weight.

### HPLC Purification

The crude thioacetal reaction solution was diluted to 1000 ml with water and then loaded, via injection pump, directly onto a preparative HPLC system (Shimadzu LC-8A Binary Systems) using a Luna Phenyl-Hexyl RP-HPLC column (Phenomenex Inc.; 5 μm, 100 Å; 250×21.2 mm). The running buffers used were A: 0.1% TFA/H₂O and B: 0.1% TFA/ACN). The initial loading was done at 20% B, with 5 min isocratic wash after loading, then set to 25% B for equilibration. The sample was eluted using a linear 25-45% B gradient over 60 min, at a flow of 25 mL/min, with column heating set at 50° C. Fractions that were determined to contain the desired product (analysis by LC-MS) were pooled, frozen and lyophilized to give a white amorphous solid product, as the TFA salt of Example 2. The purity assessed by RP-HPLC 1 was found to be >95%, with the observed molecular weight of 5308.6 Dalton; matching the theoretical calculated molecular weight of 5307.9 Dalton.

### Example 3

Example 3 is a polypeptide represented by the following description (SEQ ID NO:107) HOOC—(CH₂)₁₈—CO-(γGlu)-EKEKEKG-PEG24-EK-βAla-RSS[CFGKRIDRIGHQSGLGC]PSFRHGGKSSGAPPPS-NH₂

### (Thioacetal Linkage)

Below is a depiction of the structure of Example 3 using the standard single letter code for L-Amino Acids except for the γ-Glutamic, Glycine at position 8, βAlanine, and Cysteine residues where the structures of the residues have been expanded.

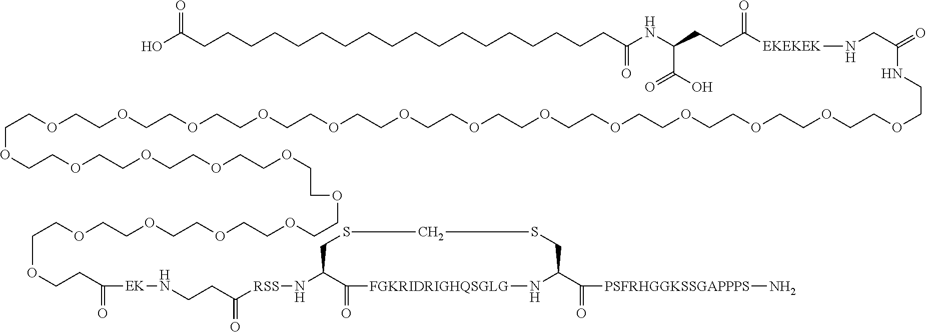

The primary peptide sequence of Example 3 was synthesized in a substantially similar manner as Examples 1 and 2 with a non-natural β-Alanine (βAla) residue which was incorporated using Fmoc-βAla-OH (ChemImpex International Inc.). An exception to the synthetic method was the coupling of Fmoc-N-amido-Peg24-OH which required a pause in the automated synthesis protocol. The PEG24 residue coupling was accomplished by the manual addition of 1.5-fold excess of Fmoc-N-amido-PEG24-OH (BroadPharm) solution which was pre-activated (2 min) with diisopropylcarbodiimide (DIC) and ethyl-cyano(hydroxyamino)acetate (Oxyma) (1:1.2:1; PEG24:DIC:Oxyma) in 3 mL DMF. The solution was added via transfer pipet directly to the Symphony-X reaction vessel containing the peptidyl-resin. The reaction time for the PEG24 coupling was 18 hours, after which point the resin was washed 3× with DMF and a Kaiser test was performed to ensure coupling completion. The PEG24 coupling process is repeated as necessary if a positive Kaiser test is noted. The automated methods were resumed to complete the synthesis of the rest of sequence and the FA was coupled as noted in Example 1. The cleavage, disulfide linkage formation, thioacetal linkage formation and purification were done as previously described in Examples 1 and 2. The purity assessed by RP-HPLC was found to be >95%, with the observed molecular weight of 6475.0 Dalton; matching the theoretical calculated molecular weight of 6475.4 Dalton.

### Example 4

Example 4 is a polypeptide represented by the following description (SEQ ID NO:144) HOOC—(CH₂)₁₈—CO-(γGlu)-EKEKEKG-PEG24-EK-βAla-RSS[CFGKRIDRIGHQSGLGC]PSFRHGSPSSGAPPPS-NH₂

### (Thioacetal Linkage)

Below is a depiction of the structure of Example 4 using the standard single letter code for L-Amino Acids except for the γ-Glutamic, Glycine at position 8, βAlanine, and Cysteine residues where the structures of the residues have been expanded.

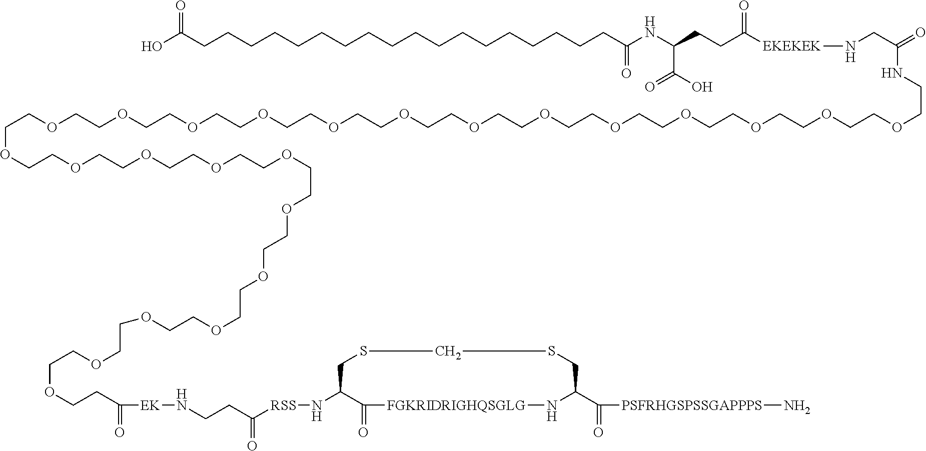

Example 4 was synthesized in a substantially similar manner as Example 3. The purity assessed by RP-HPLC for Example 4 was found to be >95%, with the observed molecular weight of 6474.6 Dalton; matching the theoretical calculated molecular weight of 6474.4 Dalton.

### Example 5

Example 5 is a polypeptide represented by the following description (SEQ ID NO:146) HOOC—(CH₂)₁₈—CO-(γGlu)-EKEKEKGEKPRSS[CFGKRIDRIGHYSGLGC]PSFRHGSPSSGAPPPS-NH₂

### (Thioacetal Linkage)

Below is a depiction of the structure of Example 5 using the standard single letter code for L-Amino Acids except for the γ-Glutamic and Cysteine residues where the structures of the residues have been expanded.

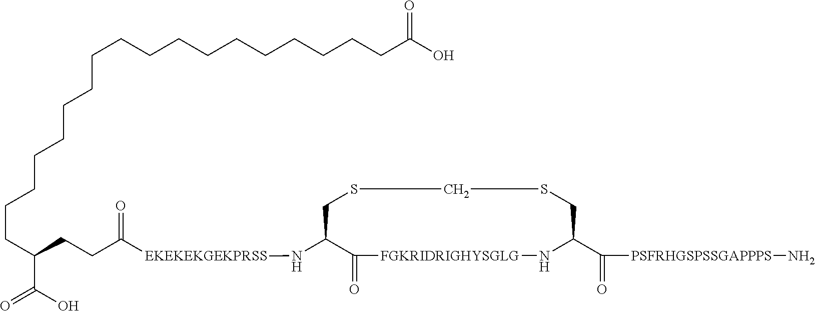

Example 5 was synthesized in a substantially similar manner as described for Example 2. The purity assessed by RP-HPLC for Example 5 was found to be >95%, with the observed molecular weight of 5406.8 Dalton; matching the theoretical calculated molecular weight of 5407.1 Dalton.

### Example 6

Example 6 is a polypeptide represented by the following description (SEQ ID NO:158) HOOC—(CH₂)₁₈—CO-(γGlu)-EKEKEKG-PEG24-EK-βAla-RSS[CFGGKIDRIGHYSGLGC]PSFRHGSPSSGAPPPS-NH₂

### (Thioacetal Linkage)

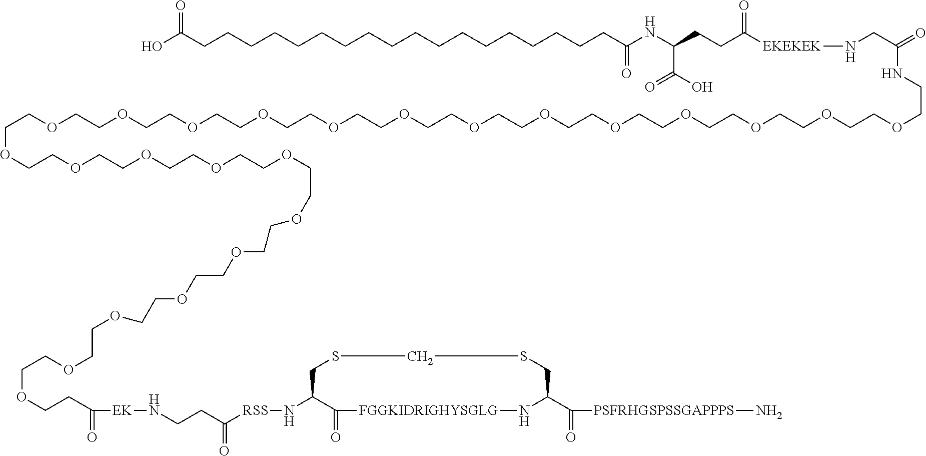

Example 6 was synthesized in a substantially similar manner as described for Example 3. The purity assessed by RP-HPLC for Example 6 was found to be >95%, with the observed molecular weight of 6409.6 Dalton; matching the theoretical calculated molecular weight of 6410.3 Dalton.

### Example 7

Example 7 is a polypeptide represented by the following description (SEQ ID NO:159) HOOC—(CH₂)₁₈—CO-(γGlu)-EKEKEKG-PEG24-EK-βAla-RSS[CFGGKIDRIGHQSGLGC]PSFRHGSPSSGAPPPS-NH₂

### (Thioacetal Linkage)

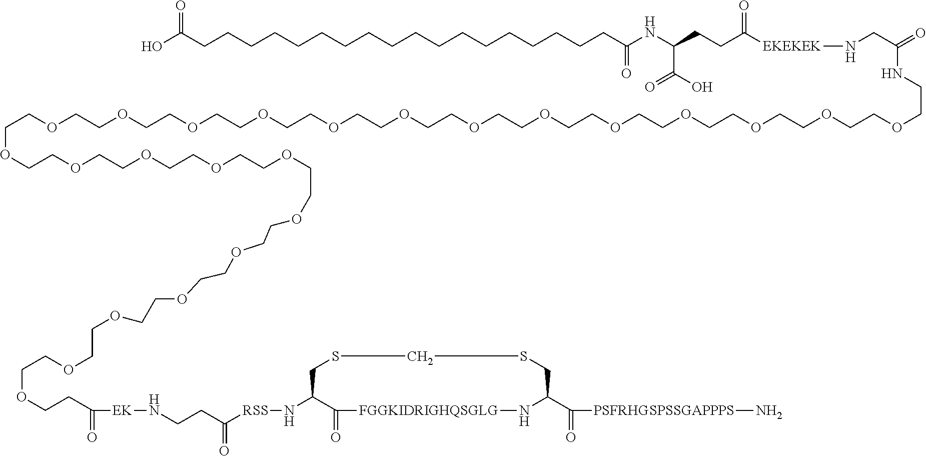

Example 7 was synthesized in a substantially similar manner as described for Example 3. The purity assessed by RP-HPLC for Example 7 was found to be >95%, with the observed molecular weight of 6375.2 Dalton, matching the theoretical calculated molecular weight of 6375.2 Dalton.

The polypeptides according to Examples 8 through Example 140 (SEQ ID NO:28-44, 46-59, 61-106, 108-143, 145, 147-157, 160-167) listed in Table 1 are prepared substantially using the procedures as described in Examples 1-3. For instance, Examples 8-16 and 18-55 (SEQ ID NO:28-36, 38-59 and 61-77) contain a disulfide linkage and are prepared substantially as described by the procedure of Example 1. Examples 17 and 56-140 (SEQ ID NO:37 and 78-106, 108-143, 145, 147-157, 160-167) contain a thioacetal linkage and are prepared substantially as described by the procedure of Example 2. Further, Examples 61-62, 75, 77, 78, 82, 84, 95, 102, 106, 109, 110, 115, 121, 124, 125, 129, 130, 132 and 133 (SEQ ID NO:83, 84, 97, 99, 100, 104, 106, 118, 125, 129, 132, 133, 138, 145, 149, 150, 154, 155, 157, 160) contain PEG24 or PEG12 which is introduced substantially as described by the procedure of Example 3, and Examples 83, 86-94, 96-100, 103-105, 107, 108, 111, 113, 116-120, 122, 123, 126 (SEQ ID NO:105, 109-117, 119-123, 126-128, 130-131, 134, 136, 139-143, 147-148, 151) contain (AEEA)₄, (AEEA)₆, or (AEEA)₈, which is introduced using standard amino acid coupling methods substantially as described in Example 2.

TABLE 1

| | | | C-C | | |
|---|---|---|---|---|---|
| | | SEQ | Disulfide | Calculated | Found |
| Example | | ID | or | MW | MW |
| Number | Polypeptide Name | NO | Thioacetal | (avg) | (avg) |
| human | SLRRSS[CFGGRMDRIGAQSGL | 1 | Disulfide | 3080.4 | 3080.5 |
| ANP | GC]NSFRY-OH | | | | |
| rat ANP | SLRRSS[CFGGRIDRIGAQSGLG | 2 | Disulfide | 3062.4 | 3062.5 |
| | CINSFRY-OH | | | | |
| rat ANP | SLRRSS[CFGGRIDRIGAQSGLG | 173 | Disulfide | 3061.4 | 3061.2 |
| amidated | C]NSFRY-NH₂ | | | | |
| 1 | HOOC-(CH₂)₁₈-CO-(γGlu)- | 45 | Disulfide | 5293.9 | 5293.4 |
| | EKEKEKGS-4Pal- | | | | |
| | RRSS[CFGGRIDRIGHQSGLGC] | | | | |
| | PSFRHGGPSSGAPPPS-NH₂ | | | | |
| 2 | HOOC-(CH₂)₁₈-CO-(γGlu)- | 60 | Thioacetal | 5307.9 | 5308.6 |
| | EKEKEKGS-4Pal- | | | | |
| | RRSS[CFGGRIDRIGHQSGLGC] | | | | |
| | PSFRHGGPSSGAPPPS-NH₂ | | | | |
| 3 | HOOC-(CH₂)₁₈-CO-(γGlu)- | 107 | Thioacetal | 6475.4 | 6475.0 |
| | EKEKEKG-PEG24-EK-βAla- | | | | |
| | RSS[CFGKRIDRIGHQSGLGC]PS | | | | |
| | FRHGGKSSGAPPPS-NH₂ | | | | |
| 4 | HOOC-(CH₂)₁₈-CO-(γGlu)- | 144 | Thioacetal | 6474.4 | 6474.6 |
| | EKEKEKG-PEG24-EK-βAla- | | | | |
| | RSS[CFGKRIDRIGHQSGLGC]PS | | | | |
| | FRHGSPSSGAPPPS-NH₂ | | | | |
| 5 | HOOC-(CH₂)₁₈-CO-(γGlu)- | 146 | Thioacetal | 5407.1 | 5406.8 |
| | EKEKEKGEKPRSS[CFGKRIDRI | | | | |
| | GHYSGLGCJPSFRHGSPSSGAPP | | | | |
| | PS-NH₂ | | | | |
| 6 | HOOC-(CH₂)₁₈-CO-(γGlu)- | 158 | Thioacetal | 6410.3 | 6409.6 |
| | EKEKEKG-PEG24-EK-βAla- | | | | |
| | RSS[CFGGKIDRIGHYSGLGC]PS | | | | |
| | FRHGSPSSGAPPPS-NH₂ | | | | |
| 7 | HOOC-(CH₂)₁₈-CO-(γGlu)- | 159 | Thioacetal | 6375.2 | 6375.2 |
| | EKEKEKG-PEG24-EK-βAla- | | | | |
| | RSS[CFGGKIDRIGHQSGLGC]PS | | | | |
| | FRHGSPSSGAPPPS-NH₂ | | | | |
| 8 | HOOC-(CH₂)₁₈-CO-(γGlu)- | 28 | Disulfide | 4343.9 | 4344.0 |
| | EKEKEKGSLRRSS[CFGGRIDRI | | | | |
| | GAQSGLGCINSFRY-NH₂ | | | | |
| 9 | HOOC-(CH₂)₁₈-CO-(γGlu)- | 29 | Disulfide | 4328.9 | 4329.0 |
| | EKEKEKGSLRRSS[CFGGRIDRI | | | | |
| | GAQSGLGC]NSFR-4Pal-NH₂ | | | | |
| 10 | HOOC-(CH₂)₁₈-CO-(γGlu)- | 30 | Disulfide | 4364.0 | 4363.8 |
| | EKEKEKGSLRRSS[CFGGRIDRI | | | | |
| | GA-4Pal-SGLGC]NSFRY-NH₂ | | | | |
| 11 | HOOC-(CH₂)₁₈-CO-(γGlu)- | 31 | Disulfide | 4379.0 | 4378.4 |
| | EKEKEKGS-4Pal- | | | | |
| | RRSS[CFGGRIDRIGAQSGLGC] | | | | |
| | NSFRY-NH₂ | | | | |
| 12 | HOOC-(CH₂)₁₈-CO-(γGlu)- | 32 | Disulfide | 4349.0 | 4348.4 |
| | EKEKEKGSLRRSS[CFGGRIDRI | | | | |
| | GA-4Pal-SGLGCJNSFR-4Pal-NH₂ | | | | |
| 13 | HOOC-(CH₂)₁₈-CO-(γGlu)- | 33 | Disulfide | 4435.1 | 4434.6 |
| | EKEKEKGSLRRSS[CF-4Pal- | | | | |
| | GRIDRIGAQSGLGC]NSFRY- | | | | |
| | NH₂ | | | | |
| 14 | HOOC-(CH₂)₁₈-CO-(γGlu)- | 34 | Disulfide | 4399.0 | 4398.8 |
| | EKEKEKGS-4Pal- | | | | |
| | RRSS[CFGGRIDRIGA-4Pal- | | | | |
| | SGLGC]NSFRY-NH₂ | | | | |
| 15 | HOOC-(CH₂)₁₈-CO-(γGlu)- | 35 | Disulfide | 4363.9 | 4363.8 |
| | EKEKEKGS-4Pal- | | | | |
| | RRSS[CFGGRIDRIGAQSGLGC] | | | | |
| | NSFR-4Pal-NH₂ | | | | |
| 16 | HOOC-(CH₂)₁₈-CO-(γGlu)- | 36 | Disulfide | 4502.1 | 4501.8 |
| | EKEKEKGSHRRSS[CFHGRIDRI | | | | |
| | HAQSGLGC]NSFRH-NH₂ | | | | |
| 17 | HOOC-(CH₂)₁₈-CO-(γGlu)- | 37 | Thioacetal | 4359.0 | 4359.3 |
| | EKEKEKGSLRRSS[CFGGRIDRI | | | | |
| | GAQSGLGCINSFRY-OH | | | | |
| 18 | HOOC-(CH₂)₁₈-CO-(γGlu)- | 38 | Disulfide | 4324.9 | 4324.6 |
| | EKEKEKGSHRRSS[CFGGRIDRI | | | | |
| | GAQSGLGC]PSFRH-NH₂ | | | | |
| 19 | HOOC-(CH₂)₁₈-CO-(γGlu)- | 39 | Disulfide | 5171.8 | 5171.9 |
| | EKEKEKGS-4Pal- | | | | |
| | KRSS[CFGGKIDRIGAQSGLGC] | | | | |
| | PSFRHGGPSSGAPPPS-NH₂ | | | | |
| 20 | HOOC-(CH₂)₁₈-CO-(γGlu)- | 40 | Disulfide | 4347.0 | 4346.4 |
| | EKEKEKGS-4Pal- | | | | |
| | RRSS[CFGGRIDRIGAQSGLGC] | | | | |
| | PSFR-4Pal-NH₂ | | | | |
| 21 | HOOC-(CH₂)₁₈-CO-(γGlu)- | 41 | Disulfide | 5227.9 | 5227.2 |
| | EKEKEKGS-4Pal- | | | | |
| | RRSS[CFGGRIDRIGAQSGLGC] | | | | |
| | PSFRHGGPSSGAPPPS-NH₂ | | | | |
| 22 | HOOC-(CH₂)₁₈-CO-(γGlu)- | 42 | Disulfide | 4335.9 | 4335.8 |
| | EKEKEKGS-4Pal- | | | | |
| | RRSS[CFGGRIDRIGAQSGLGC] | | | | |
| | PSFRH-NH₂ | | | | |
| 23 | HOOC-(CH₂)₁₈-CO-(γGlu)- | 43 | Disulfide | 5238.9 | 5238.8 |
| | EKEKEKGS-4Pal- | | | | |
| | RRSS[CFGGRIDRIGAQSGLGC] | | | | |
| | PSFR-4Pal-GGPSSGAPPPS-NH₂ | | | | |
| 24 | HOOC-(CH₂)₁₈-CO-(γGlu)- | 44 | Disulfide | 4413.0 | 4413.0 |
| | EKEKEKGS-4Pal- | | | | |
| | RRSS[CFGGRIDRIGHQSGLGC] | | | | |
| | PSFR-4Pal-NH₂ | | | | |
| 25 | HOOC-(CH₂)₁₈-CO-(γGlu)- | 46 | Disulfide | 5791.5 | 5790.6 |
| | EKEKEKGS-4Pal- | | | | |
| | RRSS[CFGGRIDRIGHQSGLGC] | | | | |
| | PSFRHRITAREDKQGYA-NH₂ | | | | |
| 26 | HOOC-(CH₂)₁₈-CO-(γGlu)- | 47 | Disulfide | 5757.4 | 5756.8 |
| | EKEKEKGS-4Pal- | | | | |
| | RRSS[CFGGRIDRIGHQSGLGC] | | | | |
| | PSFRHRITAREDKQGEA-NH₂ | | | | |
| 27 | HOOC-(CH₂)₁₈-CO-(γGlu)- | 48 | Disulfide | 5304.9 | 5305.3 |
| | EKEKEKGS-4Pal- | | | | |
| | RRSS[CFGGRIDRIGHQSGLGC] | | | | |
| | PSFR-4Pal-GGPSSGAPPPS-NH₂ | | | | |
| 28 | HOOC-(CH₂)₁₈-CO-(γGlu)- | 49 | Disulfide | 5336.0 | 5337.0 |
| | EKEKEKGE-4Pal- | | | | |
| | RRSS[CFGGRIDRIGHQSGLGC] | | | | |
| | PSFRH-GGPSSGAPPPS-NH₂ | | | | |
| 29 | HOOC-(CH₂)₁₈-CO-(γGlu)- | 50 | Disulfide | 5235.9 | 5236.5 |
| | EKEKEKGS-4Pal- | | | | |
| | RRSS[CFGGRIGRIGHQSGLGC] | | | | |
| | PSFRHGGPSSGAPPPS-NH₂ | | | | |
| 30 | HOOC-(CH₂)₁₈-CO-(γGlu)- | 51 | Disulfide | 5237.9 | 5237.4 |
| | EKEKEKGS-4Pal- | | | | |
| | KRSS[CFGGKIDRIGHQSGLGC] | | | | |
| | PSFRHGGPSSGAPPPS-NH₂ | | | | |
| 31 | HOOC-(CH₂)₁₈-CO-(γGlu)- | 52 | Disulfide | 5186.8 | 5186.0 |
| | EKEKEKGS-4Pal- | | | | |
| | KRSS[CFGGKIDRIG-Dap- | | | | |
| | QSGLGC]PSFRHGGPSSGAPPPS | | | | |
| | -NH₂ | | | | |
| 32 | HOOC-(CH₂)₁₈-CO-(γGlu)- | 53 | Disulfide | 5352.0 | 5351.2 |
| | EKEKEKGE-4Pal- | | | | |
| | KRSS[CFGGKIDRIGHQSGLGC] | | | | |
| | PSFRHGPSSGAPPPSE-NH₂ | | | | |
| 33 | HOOC-(CH₂)₁₈-CO-(γGlu)- | 54 | Disulfide | 5180.8 | 5180.0 |
| | EKEKEKGS-4Pal- | | | | |
| | KRSS[CFGGKIDRIGHQSGLGC] | | | | |
| | PSFRHGPSSGAPPPS-NH₂ | | | | |
| 34 | HOOC-(CH₂)₁₈-CO-(γGlu)- | 55 | Disulfide | 5261.0 | 5260.8 |
| | EKEKEKGS-4Pal- | | | | |
| | KRSS[CFGGKIDRIGHQSGLGC] | | | | |
| | PSFRHKITAKEDE-NH₂ | | | | |
| 35 | HOOC-(CH₂)₁₈-CO-(γGlu)- | 56 | Disulfide | 5265.9 | 5265.0 |
| | EKEKEKGS-4Pal- | | | | |
| | KRSS[CFGGRIDRIGHQSGLGC] | | | | |
| | PSFRHGGPSSGAPPPS-NH₂ | | | | |
| 36 | HOOC-(CH₂)₁₈-CO-(γGlu)- | 57 | Disulfide | 5265.9 | 5265.2 |
| | EKEKEKGS-4Pal- | | | | |
| | RRSS[CFGGKIDRIGHQSGLGC] | | | | |
| | PSFRHGGPSSGAPPPS-NH₂ | | | | |
| 37 | HOOC-(CH₂)₁₈-CO-(γGlu)- | 58 | Disulfide | 5236.9 | 5236.2 |
| | EKEKEKGS-4Pal- | | | | |
| | RRSS[CFGGRIDRIGHQSGLGC] | | | | |
| | PSFRH-GPSSGAPPPS-NH₂ | | | | |
| 38 | HOOC-(CH₂)₁₈-CO-(γGlu)- | 59 | Disulfide | 5329.0 | 5328.4 |
| | EKEKEKGS-4Pal- | | | | |
| | RRSS[CFGGRIDRIGHYSGLGC] | | | | |
| | PSFRHGGPSSGAPPPS-NH₂ | | | | |
| 39 | HOOC-(CH₂)₁₈-CO-(γGlu)- | 61 | Disulfide | 5124.7 | 5124.0 |
| | EKEKEKGS-4Pal- | | | | |
| | RRSS[CFGGRIDRIGHQSGLGC] | | | | |
| | PSFRHSGAPPPSE-NH₂ | | | | |
| 40 | HOOC-(CH₂)₁₈-CO-(γGlu)- | 62 | Disulfide | 5288.9 | 5288.5 |
| | EKEKEKGEKERSS[CFGGRIDRI | | | | |
| | GHQSGLGC]PSFRHGGPSSGAP | | | | |
| | PPS-NH₂ | | | | |
| 41 | HOOC-(CH₂)₁₈-CO-(γGlu)- | 63 | Disulfide | 4902.5 | 4902.1 |
| | EKEKEKGRSS[CFGGRIDRIGHQ | | | | |
| | SGLGC]PSFRHGGPSSGAPPPS- | | | | |
| | NH₂ | | | | |
| 42 | HOOC-(CH₂)₁₈-CO-(γGlu)- | 64 | Disulfide | 5194.8 | 5194.2 |
| | EKEKEKGEKGRSS[CFGGRIDRI | | | | |
| | GHYSGLGC]PSFRH- | | | | |
| | GPSSGAPPPS-NH₂ | | | | |
| 43 | HOOC-(CH₂)₁₈-CO-(γGlu)- | 65 | Disulfide | 4909.5 | 4909.2 |
| | EKEKEKGRSS[CFGGKIDRIGHY | | | | |
| | SGLGC]PSFRHGGPSSGAPPPS- | | | | |
| | NH₂ | | | | |
| 44 | HOOC-(CH₂)₁₈-CO-(γGlu)- | 66 | Disulfide | 5295.9 | 5295.5 |
| | EKEKEKGEKERSS[CFGGKIDRI | | | | |
| | GHYSGLGC]PSFRHGGPSSGAP | | | | |
| | PPS-NH₂ | | | | |
| 45 | HOOC-(CH₂)₁₈-CO-(γGlu)- | 67 | Disulfide | 5223.9 | 5223.5 |
| | EKEKEKGEKGRSS[CFGGKIDRI | | | | |
| | GHYSGLGC]PSFRHGGPSSGAP | | | | |
| | PPS-NH₂ | | | | |
| 46 | HOOC-(CH₂)₁₈-CO-(γGlu)- | 68 | Disulfide | 5136.8 | 5136.0 |
| | EKEKEKGRRSS[CFGGKIDRIGH | | | | |
| | YSGLGC]PSFRHKGPSSGAPPPS | | | | |
| | -NH₂ | | | | |
| 47 | HOOC-(CH₂)₁₈-CO-(γGlu)- | 69 | Disulfide | 4980.6 | 4980.0 |
| | EKEKEKGRSS[CFGGKIDRIGHY | | | | |
| | SGLGC]PSFRHKGPSSGAPPPS- | | | | |
| | NH₂ | | | | |
| 48 | HOOC-(CH₂)₁₈-CO-(γGlu)- | 70 | Disulfide | 4973.6 | 4973.2 |
| | (APPPS)G]- | | | | |
| | EKERSS[CFGGKIDRIGHYSGLG | | | | |
| | C]PSFRHGGPSSGAPPPS-NH₂ | | | | |
| 49 | HOOC-(CH₂)₁₈-CO-(γGlu)- | 71 | Disulfide | 5323.9 | 5323.5 |
| | EKEKEKGEKERSS[CFGGRIDRI | | | | |
| | GHYSGLGCJPSFRHGGPSSGAP | | | | |
| | PPS-NH₂ | | | | |
| 50 | HOOC-(CH₂)₁₈-CO-(γGlu)- | 72 | Disulfide | 5380.0 | 5379.0 |
| | EKEKEKGEKGRSS[CFGGRIDRI | | | | |
| | GHYSGLGC]PSFRHKGGPSSGA | | | | |
| | PPPS-NH₂ | | | | |
| 51 | HOOC-(CH₂)₁₈-CO-(γGlu)- | 73 | Disulfide | 5217.9 | 5217.6 |
| | EKEKEKGEKGRSS[CFGGRIDRI | | | | |
| | GHYSGLGCJPSLRHGGPSSGAP | | | | |
| | PPS-NH₂ | | | | |
| 52 | HOOC-(CH₂)₁₈-CO-(γGlu)- | 74 | Disulfide | 5214.9 | 5214.3 |
| | EKEKEKGEKGRSS[CFGGKIDRI | | | | |
| | GKYSGLGCJPSFRHGGPSSGAP | | | | |
| | PPS-NH₂ | | | | |
| 53 | HOOC-(CH₂)₁₈-CO-(γGlu)- | 75 | Disulfide | 5185.9 | 5185.2 |
| | EKEKEKGEKGRSS[CFGGRIDRI | | | | |
| | GKYSGLGC]PSFRHGPSSGAPPP | | | | |
| | S-NH₂ | | | | |
| 54 | HOOC-(CH₂)₁₈-CO-(γGlu)- | 76 | Disulfide | 5265.9 | 5265.6 |
| | EKEKEKGEK-βAla- | | | | |
| | RSS[CFGGRIDRIGHYSGLGC]PS | | | | |
| | FRHGGPSSGAPPPS-NH₂ | | | | |
| 55 | HOOC-(CH₂)₁₈-CO-(γGlu)- | 77 | Disulfide | 5291.9 | 5291.3 |
| | EKEKEKGEKPRSS[CFGGRIDRI | | | | |
| | GHYSGLGC]PSFRHGGPSSGAP | | | | |
| | PPS-NH₂ | | | | |
| 56 | HOOC-(CH₂)₁₈-CO-(γGlu)- | 78 | Thioacetal | 5279.9 | 5279.4 |
| | EKEKEKGEK-βAla- | | | | |
| | RSS[CFGGRIDRIGHYSGLGC]PS | | | | |
| | FRHGGPSSGAPPPS-NH₂ | | | | |
| 57 | HOOC-(CH₂)₁₈-CO-(γGlu)- | 79 | Thioacetal | 5306.0 | 5305.3 |
| | EKEKEKGEKPRSS[CFGGRIDRI | | | | |
| | GHYSGLGC]PSFRHGGPSSGAP | | | | |
| | PPS-NH₂ | | | | |
| 58 | HOOC-(CH₂)₁₈-CO-(γGlu)- | 80 | Thioacetal | 5279.9 | 5279.4 |
| | EKEKEKGKE-βAla- | | | | |
| | RSS[CFGGRIDRIGHYSGLGC]PS | | | | |
| | FRHGGPSSGAPPPS-NH₂ | | | | |
| 59 | HOOC-(CH₂)₁₈-CO-(γGlu)- | 81 | Thioacetal | 5337.0 | 5336.4 |
| | EKEKEKGEKGRSS[CFGKRIDRI | | | | |
| | GHYSGLGCJPSFRHGGPSSGAP | | | | |
| | PPS-NH₂ | | | | |
| 60 | HOOC-(CH₂)₁₈-CO-(γGlu)- | 82 | Thioacetal | 5222.9 | 5222.0 |
| | EKEKEKGEK-ßAla- | | | | |
| | RSS[CFGGRIDRIGHYSGLGC]PS | | | | |
| | FRHGPSSGAPPPS-NH₂ | | | | |
| 61 | HOOC-(CH₂)₁₈-CO-(γGlu)- | 83 | Thioacetal | 6436.3 | 6436.8 |
| | EKEKEKG-PEG24-S-4Pal- | | | | |
| | RRSS[CFGGRIDRIGHQSGLGC] | | | | |
| | PSFRHGGPSSGAPPPS-NH₂ | | | | |
| 62 | HOOC-(CH₂)₁₈-CO-(γGlu)- | 84 | Thioacetal | 6408.3 | 6409.0 |
| | EKEKEKG-PEG24-EK-βAla- | | | | |
| | RSS[CFGGRIDRIGHYSGLGC]PS | | | | |
| | FRHGGPSSGAPPPS-NH₂ | | | | |
| 63 | HOOC-(CH₂)₁₈-CO-(γGlu)- | 85 | Thioacetal | 5245.9 | 5245.8 |
| | EKEKEKGEK-ßAla- | | | | |
| | RSS[CFGGRIDRIGHYSGLGC]PS | | | | |
| | LRHGGPSSGAPPPS-NH₂ | | | | |
| 64 | HOOC-(CH₂)₁₈-CO-(γGlu)- | 86 | Thioacetal | 5309.0 | 5309.0 |
| | EKEKEKGSK-βAla- | | | | |
| | RSS[CFGKRIDRIGHYSGLGC]PS | | | | |
| | FRHGGPSSGAPPPS-NH₂ | | | | |
| 65 | HOOC-(CH₂)₁₈-CO-(γGlu)- | 87 | Thioacetal | 5237.9 | 5238.1 |
| | EKEKEKGSK-ßAla- | | | | |
| | RSS[CFGGRIDRIGHYSGLGC]PS | | | | |
| | FRHGGPSSGAPPPS-NH₂ | | | | |
| 66 | HOOC-(CH₂)₁₈-CO-(γGlu)- | 88 | Thioacetal | 5210.8 | 5210.1 |
| | EKEKEKGS-4Pal- | | | | |
| | RRSS[CFGGRIDRIGHQSGLGC] | | | | |
| | PSFRHGG--SSGAPPPS-NH₂ | | | | |
| 67 | HOOC-(CH₂)₁₈-CO-(γGlu)- | 89 | Thioacetal | 5339.0 | 5339.0 |
| | EKEKEKGS-4Pal- | | | | |
| | RRSS[CFGGRIDRIGHQSGLGC] | | | | |
| | PSFRHGGKSSGAPPPS-NH₂ | | | | |
| 68 | HOOC-(CH₂)₁₈-CO-(γGlu)- | 90 | Thioacetal | 5444.2 | 5444.0 |
| | EKEKEKGS-4Pal- | | | | |
| | RRSS[CFGGRIDRIGHQSGLGC] | | | | |
| | PSFRHGGPPS-Aib-KPPPK-NH₂ | | | | |
| 69 | HOOC-(CH₂)₁₈-CO-(γGlu)- | 91 | Thioacetal | 5279.9 | 5279.4 |
| | EKEKEKGS-4Pal- | | | | |
| | RRSS[CFGGRIDRIGHQSGLGC] | | | | |
| | PSFRHGGPSSGAPPPS-NH₂ | | | | |
| 70 | HOOC-(CH₂)₁₈-CO-(γGlu)- | 92 | Thioacetal | 5251.9 | 5251.2 |
| | EKEKEKGEK-ßAla- | | | | |
| | RSS[CFGGRIDRIGHYSGLGC]PS | | | | |
| | FRHGGPSSGAPPPS-NH₂ | | | | |
| 71 | HOOC-(CH₂)₁₈-CO-(γGlu)- | 93 | Thioacetal | 5416.2 | 5416.0 |
| | EKEKEKGS-4Pal- | | | | |
| | RRSS[CFGGRIDRIGHQSGLGC] | | | | |
| | PSFRHGGPPS-Aib-KPPPK-NH₂ | | | | |
| 72 | HOOC-(CH₂)₁₈-CO-(γGlu)- | 94 | Thioacetal | 5310.9 | 5311.2 |
| | EKEKEKGS-4Pal- | | | | |
| | RRSS[CFGGRIDRIGHQSGLGC] | | | | |
| | PSFRHGGKSSGAPPPS-NH₂ | | | | |
| 73 | HOOC-(CH₂)₁₄-CO-(γGlu)- | 95 | Thioacetal | 5254.9 | 5254.2 |
| | EKEKEKGEK-ßAla- | | | | |
| | RSS[CFGGRIDRIGHYSGLGC]PS | | | | |
| | FRHGGKSSGAPPPS-NH₂ | | | | |
| 74 | HOOC-(CH₂)₁₈-CO-(γGlu)- | 96 | Thioacetal | 5311.0 | 5310.6 |
| | EKEKEKGEK-ßAla- | | | | |
| | RSS[CFGGRIDRIGHYSGLGC]PS | | | | |
| | FRHGGKSSGAPPPS-NH₂ | | | | |
| 75 | HOOC-(CH₂)₁₈-CO-(γGlu)- | 97 | Thioacetal | 6467.3 | 6467.0 |
| | EKEKEKG-PEG24-S-4Pal- | | | | |
| | RRSS[CFGGRIDRIGHQSGLGC] | | | | |
| | PSFRHGGKSSGAPPPS-NH₂ | | | | |
| 76 | HOOC-(CH₂)₁₈-CO-(γGlu)- | 98 | Thioacetal | 5282.9 | 5282.4 |
| | EKEKEKGEK-βAla- | | | | |
| | RSS[CFGGRIDRIGHYSGLGC]PS | | | | |
| | FRHGGKSSGAPPPS-NH₂ | | | | |
| 77 | HOOC-(CH₂)₁₈-CO-(γGlu)- | 99 | Thioacetal | 5910.7 | 5910.4 |
| | EKEKEKG-PEG12-EK-βAla- | | | | |
| | RSS[CFGGRIDRIGHYSGLGC]PS | | | | |
| | FRHGGKSSGAPPPS-NH₂ | | | | |
| 78 | HOOC-(CH₂)₁₈-CO-(γGlu)- | 100 | Thioacetal | 6439.3 | 6439.0 |
| | EKEKEKG-PEG24-EK-βAla- | | | | |
| | RSS[CFGGRIDRIGHYSGLGC]PS | | | | |
| | FRHGGKSSGAPPPS-NH₂ | | | | |
| 79 | HOOC-(CH₂)₁₈-CO-(γGlu)- | 101 | Thioacetal | 5382.1 | 5381.6 |
| | EKEKEKGS-4Pal- | | | | |
| | RRSS[CFGKRIDRIGHQSGLGC] | | | | |
| | PSFRHGGKSSGAPPPS-NH₂ | | | | |
| 80 | HOOC-(CH₂)₁₈-CO-(γGlu)- | 102 | Thioacetal | 5382.1 | 5381.0 |
| | EKEKEKGS-4Pal- | | | | |
| | RRSS[CFGRKIDRIGHQSGLGC] | | | | |
| | PSFRHGGKSSGAPPPS-NH₂ | | | | |
| 81 | HOOC-(CH₂)₁₈-CO-(γGlu)- | 103 | Thioacetal | 5417.1 | 5416.2 |
| | EKEKEKGS-4Pal- | | | | |
| | RRSS[CFGKRIDRIGHYSGLGC] | | | | |
| | PSFRHGGKSSGAPPPS-NH₂ | | | | |
| 82 | HOOC-(CH₂)₁₈-CO-(γGlu)- | 104 | Thioacetal | 5938.7 | 5938.2 |
| | EKEKEKG-PEG12-S-4Pal- | | | | |
| | RRSS[CFGGRIDRIGHQSGLGC] | | | | |
| | PSFRHGGKSSGAPPPS-NH₂ | | | | |
| 83 | HOOC-(CH₂)₁₈-CO-(γGlu)- | 105 | Thioacetal | 5919.6 | 5919.2 |
| | EKEKEKG-(AEEA)4-S-4Pal- | | | | |
| | RRSS[CFGGRIDRIGHQSGLGC] | | | | |
| | PSFRHGGKSSGAPPPS-NH₂ | | | | |
| 84 | HOOC-(CH₂)₁₈-CO-(γGlu)- | 106 | Thioacetal | 6572.6 | 6572.0 |
| | EKEKEKG-PEG24-S-4Pal- | | | | |
| | RRSS[CFGGRIDRIGHQSGLGC] | | | | |
| | PSFRHGGPPS-Aib-KPPPK-NH₂ | | | | |
| 85 | HOOC-(CH₂)₁₈-CO-(γGlu)- | 108 | Thioacetal | 5319.0 | 5318.6 |
| | EKEKEKGEK-ßAla- | | | | |
| | RSS[CFGKRIDRIGHQSGLGC]PS | | | | |
| | FRHGGKSSGAPPPS-NH₂ | | | | |
| 86 | HOOC-(CH₂)₁₈-CO-(γGlu)- | 109 | Thioacetal | 6209.9 | 6209.0 |
| | EKEKEKG-(AEEA)6-S-4Pal- | | | | |
| | RRSS[CFGGRIDRIGHQSGLGC] | | | | |
| | PSFRHGGKSSGAPPPS-NH₂ | | | | |
| 87 | HOOC-(CH₂)₁₈-CO-(γGlu)- | 110 | Thioacetal | 6571.4 | 6570.6 |
| | EKEKEKG-(AEEA)8-S-4Pal- | | | | |
| | RRSS[CFGKRIDRIGHQSGLGC] | | | | |
| | PSFRHGGKSSGAPPPS-NH₂ | | | | |
| 88 | HOOC-(CH₂)₁₈-CO-(γGlu)- | 111 | Thioacetal | 5927.7 | 5927.4 |
| | EKEKEKG-(AEEA)4-EK-ßAla- | | | | |
| | RSS[CFGKRIDRIGHQSGLGC]PS | | | | |
| | FRHGGKSSGAPPPS-NH₂ | | | | |
| 89 | HOOC-(CH₂)₁₈-CO-(γGlu)- | 112 | Thioacetal | 6218.0 | 6217.6 |
| | EKEKEKG-(AEEA)6-EK-βAla- | | | | |
| | RSS[CFGKRIDRIGHQSGLGC]PS | | | | |
| | FRHGGKSSGAPPPS-NH₂ | | | | |
| 90 | HOOC-(CH₂)₁₈-CO-(γGlu)- | 113 | Thioacetal | 5679.4 | 5678.4 |
| | (AEEA)8-EK-ßAla- | | | | |
| | RSS[CFGKRIDRIGHQSGLGC]PS | | | | |
| | FRHGGKSSGAPPPS-NH₂ | | | | |
| 91 | HOOC-(CH₂)₁₈-CO-(γGlu)- | 114 | Thioacetal | 6535.3 | 6534.9 |
| | EKEKEKG-(AEEA)8-S-4Pal- | | | | |
| | RRSS[CFGGRIDRIGHYSGLGC] | | | | |
| | PSFRHGGKSSGAPPPS-NH₂ | | | | |
| 92 | HOOC-(CH₂)₁₈-CO-(γGlu)- | 115 | Thioacetal | 6245.0 | 6245.0 |
| | EKEKEKG-(AEEA)6-S-4Pal- | | | | |
| | RRSS[CFGGRIDRIGHYSGLGC] | | | | |
| | PSFRHGGKSSGAPPPS-NH₂ | | | | |
| 93 | HOOC-(CH₂)₁₈-CO-(γGlu)- | 116 | Thioacetal | 6472.2 | 6471.9 |
| | EKEKEKG-(AEEA)8-EK-ßAla- | | | | |
| | RSS[CFGGRIDRIGHYSGLGC]PS | | | | |
| | FRHGGKSSGAPPPS-NH₂ | | | | |
| 94 | HOOC-(CH₂)₁₈-CO-(γGlu)- | 117 | Thioacetal | 6181.9 | 6181.6 |
| | EKEKEKG-(AEEA)6-EK-βAla- | | | | |
| | RSS[CFGGRIDRIGHYSGLGC]PS | | | | |
| | FRHGGKSSGAPPPS-NH₂ | | | | |
| 95 | HOOC-(CH₂)₁₈-CO-(γGlu)- | 118 | Thioacetal | 6502.4 | 6502.3 |
| | EKEKEKG-PEG24-S-4Pal- | | | | |
| | RRSS[CFGGRIDRIGHYSGLGC] | | | | |
| | PSFRHGGKSSGAPPPS-NH₂ | | | | |
| 96 | HOOC-(CH₂)₁₈-CO-(γGlu)- | 119 | Thioacetal | 6244.0 | 6243.5 |
| | EKEKEKG-(AEEA)6-S-4Pal- | | | | |
| | RRSS[CFGGRIDRIGHYSGLGC] | | | | |
| | PSFRHGSPSSGAPPPS-NH₂ | | | | |
| 97 | HOOC-(CH₂)₁₈-CO-(γGlu)- | 120 | Thioacetal | 6350.2 | 6350.0 |
| | EKEKEKG-(AEEA)6-S-4Pal- | | | | |
| | RRSS[CFGGRIDRIGHYSGLGC] | | | | |
| | PSFRHGGPPS-Aib-KPPPK-NH₂ | | | | |
| 98 | HOOC-(CH₂)₁₈-CO-(γGlu)- | 121 | Thioacetal | 6180.9 | 6180.0 |
| | EKEKEKG-(AEEA)6-EK-βAla- | | | | |
| | RSS[CFGGRIDRIGHYSGLGC]PS | | | | |
| | FRHGSPSSGAPPPS-NH₂ | | | | |
| 99 | HOOC-(CH₂)₁₈-CO-(γGlu)- | 122 | Thioacetal | 5962.7 | 5962.4 |
| | EKEKEKG-(AEEA)4-EK-ßAla- | | | | |
| | RSS[CFGKRIDRIGHYSGLGC]PS | | | | |
| | FRHGGKSSGAPPPS-NH₂ | | | | |
| 100 | HOOC-(CH₂)₁₈-CO-(γGlu)- | 123 | Thioacetal | 6253.0 | 6252.3 |
| | EKEKEKG-(AEEA)6-EK-βAla- | | | | |
| | RSS[CFGKRIDRIGHYSGLGC]PS | | | | |
| | FRHGGKSSGAPPPS-NH₂ | | | | |
| 101 | HOOC-(CH₂)₁₈-CO-(γGlu)- | 124 | Thioacetal | 5353.0 | 5352.2 |
| | EKEKEKGEK-ßAla- | | | | |
| | RSS[CFGKRIDRIGHYSGLGC]PS | | | | |
| | FRHGSPSSGAPPPS-NH₂ | | | | |
| 102 | HOOC-(CH₂)₁₈-CO-(γGlu)- | 125 | Thioacetal | 6510.4 | 6510.0 |
| | EKEKEKG-PEG24-EK-βAla- | | | | |
| | RSS[CFGKRIDRIGHYSGLGC]PS | | | | |
| | FRHGGKSSGAPPPS-NH₂ | | | | |
| 103 | HOOC-(CH₂)₁₈-CO-(γGlu)- | 126 | Thioacetal | 5961.7 | 5961.2 |
| | EKEKEKG-(AEEA)4-EK-βAla- | | | | |
| | RSS[CFGKRIDRIGHYSGLGC]PS | | | | |
| | FRHGSPSSGAPPPS-NH₂ | | | | |
| 104 | HOOC-(CH₂)₁₈-CO-(γGlu)- | 127 | Thioacetal | 6316.1 | 6316.2 |
| | EKEKEKG-(AEEA)6-S-4Pal- | | | | |
| | RRSS[CFGKRIDRIGHYSGLGC] | | | | |
| | PSFRHGGKSSGAPPPS-NH₂ | | | | |
| 105 | HOOC-(CH₂)₁₈-CO-(γGlu)- | 128 | Thioacetal | 6252.0 | 6251.4 |
| | EKEKEKG-(AEEA)6-EK-βAla- | | | | |
| | RSS[CFGKRIDRIGHYSGLGC]PS | | | | |
| | FRHGSPSSGAPPPS-NH₂ | | | | |
| 106 | HOOC-(CH₂)₁₈-CO-(γGlu)- | 129 | Thioacetal | 6509.4 | 6508.8 |
| | EKEKEKG-PEG24-EK-βAla- | | | | |
| | RSS[CFGKRIDRIGHYSGLGC]PS | | | | |
| | FRHGSPSSGAPPPS-NH₂ | | | | |
| 107 | HOOC-(CH₂)₁₈-CO-(γGlu)- | 130 | Thioacetal | 6605.4 | 6605.0 |
| | EKEKEKG-(AEEA)8-S-4Pal- | | | | |
| | RRSS[CFGKRIDRIGHYSGLGC] | | | | |
| | PSFRHGSPSSGAPPPS-NH₂ | | | | |
| 108 | HOOC-(CH₂)₁₈-CO-(γGlu)- | 131 | Thioacetal | 6542.3 | 6542.4 |
| | EKEKEKG-(AEEA)8-EK-ßAla- | | | | |
| | RSS[CFGKRIDRIGHYSGLGC]PS | | | | |
| | FRHGSPSSGAPPPS-NH₂ | | | | |
| 109 | HOOC-(CH₂)₁₈-CO-(γGlu)- | 132 | Thioacetal | 6538.5 | 6538.5 |
| | EKEKEKG-PEG24-S-4Pal- | | | | |
| | RRSS[CFGKRIDRIGHQSGLGC] | | | | |
| | PSFRHGGKSSGAPPPS-NH₂ | | | | |
| 110 | HOOC-(CH₂)₁₈-CO-(γGlu)- | 133 | Thioacetal | 6573.5 | 6573.6 |
| | EKEKEKG-PEG24-S-4Pal- | | | | |
| | RRSS[CFGKRIDRIGHYSGLGC] | | | | |
| | PSFRHGGKSSGAPPPS-NH₂ | | | | |
| 111 | HOOC-(CH₂)₁₈-CO-(γGlu)- | 134 | Thioacetal | 6159.9 | 6158.4 |
| | EKEKEKG-(AEEA)6-S-4Pal- | | | | |
| | βAla- | | | | |
| | RSS[CFGGRIDRIGHYSGLGC]PS | | | | |
| | FRHGSPSSGAPPPS-NH₂ | | | | |
| 112 | HOOC-(CH₂)₁₈-CO-(γGlu)- | 135 | Thioacetal | 5416.1 | 5416.2 |
| | EKEKEKGS-4Pal- | | | | |
| | RRSS[CFGKRIDRIGHYSGLGC] | | | | |
| | PSFRHGSPSSGAPPPS-NH₂ | | | | |
| 113 | HOOC-(CH₂)₁₈-CO-(γGlu)- | 136 | Thioacetal | 6315.1 | 6314.0 |
| | EKEKEKG-(AEEA)6-S-4Pal- | | | | |
| | RRSS[CFGKRIDRIGHYSGLGC] | | | | |
| | PSFRHGSPSSGAPPPS-NH₂ | | | | |
| 114 | HOOC-(CH₂)₁₈-CO-(γGlu)- | 137 | Thioacetal | 5379.1 | 5378.1 |
| | EKEKEKGEKPRSS[CFGKRIDRI | | | | |
| | GHYSGLGCJPSFRHGSPSSGAPP | | | | |
| | PS-NH₂ | | | | |
| 115 | HOOC-(CH₂)₁₈-CO-(γGlu)- | 138 | Thioacetal | 6572.5 | 6571.8 |
| | EKEKEKG-PEG24-S-4Pal- | | | | |
| | RRSS[CFGKRIDRIGHYSGLGC] | | | | |
| | PSFRHGSPSSGAPPPS-NH₂ | | | | |
| 116 | HOOC-(CH₂)₁₈-CO-(γGlu)- | 139 | Thioacetal | 6422.2 | 6421.8 |
| | EKEKEKG-(AEEA)6- | | | | |
| | EKPRSS[CFGKRIDRIGHYSGLG | | | | |
| | CJPSFRHSGSPSSGAPPPSG-NH₂ | | | | |
| 117 | HOOC-(CH₂)₁₈-CO-(γGlu)- | 140 | Thioacetal | 6516.3 | 6516.0 |
| | EKEKEKG-(AEEA)6-S-4Pal- | | | | |
| | RRSS[CFGKRIDRIGHYSGLGC] | | | | |
| | PSFRHGSGSPSSGAPPPSG-NH₂ | | | | |
| 118 | HOOC-(CH₂)₁₈-CO-(γGlu)- | 141 | Thioacetal | 6471.2 | 6471.0 |
| | EKEKEKG-(AEEA)8-EK-βAla- | | | | |
| | RSS[CFGGRIDRIGHYSGLGC]PS | | | | |
| | FRHGSPSSGAPPPS-NH₂ | | | | |
| 119 | HOOC-(CH₂)₁₈-CO-(γGlu)- | 142 | Thioacetal | 6217.0 | 6216.3 |
| | EKEKEKG-(AEEA)6-EK-ßAla- | | | | |
| | RSS[CFGKRIDRIGHQSGLGC]PS | | | | |
| | FRHGSPSSGAPPPS-NH₂ | | | | |
| 120 | HOOC-(CH₂)₁₈-CO-(γGlu)- | 143 | Thioacetal | 6507.3 | 6507.0 |
| | EKEKEKG-(AEEA)8-EK-ßAla- | | | | |
| | RSS[CFGKRIDRIGHQSGLGC]PS | | | | |
| | FRHGSPSSGAPPPS-NH₂ | | | | |
| 121 | HOOC-(CH₂)₁₈-CO-(γGlu)- | 145 | Thioacetal | 6438.3 | 6438.4 |
| | EKEKEKG-PEG24-EK-βAla- | | | | |
| | RSS[CFGGRIDRIGHYSGLGC]PS | | | | |
| | FRHGSPSSGAPPPS-NH₂ | | | | |
| 122 | HOOC-(CH₂)₁₈-CO-(γGlu)- | 147 | Thioacetal | 6278.0 | 6277.6 |
| | EKEKEKG-(AEEA)6- | | | | |
| | EKPRSS[CFGKRIDRIGHYSGLG | | | | |
| | CJPSFRHGSPSSGAPPPS-NH₂ | | | | |
| 123 | HOOC-(CH₂)₁₈-CO-(γGlu)- | 148 | Thioacetal | 6568.4 | 6568.0 |
| | EKEKEKG-(AEEA)8- | | | | |
| | EKPRSS[CFGKRIDRIGHYSGLG | | | | |
| | CJPSFRHGSPSSGAPPPS-NH₂ | | | | |
| 124 | HOOC-(CH₂)₁₈-CO-(γGlu)- | 149 | Thioacetal | 6679.6 | 6679.4 |
| | EKEKEKG-PEG24- | | | | |
| | EKPRSS[CFGKRIDRIGHYSGLG | | | | |
| | CJPSFRHSGSPSSGAPPPSG-NH₂ | | | | |
| 125 | HOOC-(CH₂)₁₈-CO-(γGlu)- | 150 | Thioacetal | 6535.4 | 6535.2 |
| | EKEKEKG-PEG24- | | | | |
| | EKPRSS[CFGKRIDRIGHYSGLG | | | | |
| | C]PSFRHGSPSSGAPPPS-NH₂ | | | | |
| 126 | HOOC-(CH₂)₁₈-CO-(γGlu)- | 151 | Thioacetal | 6712.9 | 6712.8 |
| | EKEKEKG-(AEEA)8- | | | | |
| | EKPRSS[CFGKRIDRIGHYSGLG | | | | |
| | C]PSFRHSGSPSSGAPPPSG-NH₂ | | | | |
| 127 | HOOC-(CH₂)₁₈-CO-(γGlu)- | 152 | Thioacetal | 5372.1 | 5371.8 |
| | EKEKEKGEKPRSS[CFGKRIDRI | | | | |
| | GHQSGLGCJPSFRHGSPSSGAPP | | | | |
| | PS-NH₂ | | | | |
| 128 | HOOC-(CH₂)₁₈-CO-(γGlu)- | 153 | Thioacetal | 5381.1 | 5380.8 |
| | EKEKEKGEK-βAla- | | | | |
| | RSS[CFGKRIDRIGHYSGLGC]PS | | | | |
| | FRHGSPSSGAPPPS-NH₂ | | | | |
| 129 | HOOC-(CH₂)₁₈-CO-(γGlu)- | 154 | Thioacetal | 5798.7 | 5798.7 |
| | EKEKEKG-PEG24-EK-βAla- | | | | |
| | RSS[CFGKRIDRIGHYSGLGC]PS | | | | |
| | FRHGGP-NH₂ | | | | |
| 130 | HOOC-(CH₂)₁₈-CO-(γGlu)- | 155 | Thioacetal | 5763.6 | 5763.8 |
| | EKEKEKG-PEG24-EK-βAla- | | | | |
| | RSS[CFGKRIDRIGHQSGLGC]PS | | | | |
| | FRHGGP-NH₂ | | | | |
| 131 | HOOC-(CH₂)₁₈-CO-(γGlu)- | 156 | Thioacetal | 5573.2 | 5572.2 |
| | EKEKEKGEKPRSS[CFGKRIDRI | | | | |
| | GHQSGLGC]PSFRHGSGSPSSG | | | | |
| | APPPSG-NH₂ | | | | |
| 132 | HOOC-(CH₂)₁₈-CO-(γGlu)- | 157 | Thioacetal | 6644.5 | 6644.0 |
| | EKEKEKG-PEG24- | | | | |
| | EKPRSS[CFGKRIDRIGHQSGLG | | | | |
| | CJPSFRHSGSPSSGAPPPSG-NH₂ | | | | |
| 133 | HOOC-(CH₂)₁₈-CO-(γGlu)- | 160 | Thioacetal | 6545.4 | 6544.8 |
| | EKEKEKG-PEG24- | | | | |
| | EKPRSS[CFGKGIDRIGHQSGLG | | | | |
| | CJPSFRHSGSPSSGAPPPSG-NH₂ | | | | |
| 134 | HOOC-(CH₂)₁₈-CO-(γGlu)- | 161 | Thioacetal | 5349.1 | 5348.4 |
| | EKEKEKGEKPRSS[CFGKRIDRI | | | | |
| | G-Orn- | | | | |
| | QSGLGCJPSFRHGSPSSGAPPPS- | | | | |
| | NH₂ | | | | |
| 135 | HOOC-(CH₂)₁₈-CO-(γGlu)- | 162 | Thioacetal | 5384.1 | 5383.8 |
| | EKEKEKGEKPRSS[CFGKRIDRI | | | | |
| | G-Orn- | | | | |
| | YSGLGCJPSFRHGSPSSGAPPPS- | | | | |
| | NH₂ | | | | |
| 136 | HOOC-(CH₂)₁₈-CO-(γGlu)- | 163 | Thioacetal | 5365.0 | 5364.6 |
| | EKEKEKGEKPRSS[CFG-Dap- | | | | |
| | RIDRIGHYSGLGCJPSFRHGSPS | | | | |
| | SGAPPPS-NH₂ | | | | |
| 137 | HOOC-(CH₂)₁₈-CO-(γGlu)- | 164 | Thioacetal | 5330.0 | 5329.8 |
| | EKEKEKGEKPRSS[CFG-Dap- | | | | |
| | RIDRIGHQSGLGCJPSFRHGSPS | | | | |
| | SGAPPPS-NH₂ | | | | |
| 138 | HOOC-(CH₂)₁₈-CO-(γGlu)- | 165 | Thioacetal | 5265.9 | 5266.1 |
| | EKEKEKGEKPRSS[CFGG-Dap- | | | | |
| | IDRIGHYSGLGCJPSFRHGSPSS | | | | |
| | GAPPPS-NH₂ | | | | |
| 139 | HOOC-(CH₂)₁₈-CO-(γGlu)- | 166 | Thioacetal | 5230.9 | 5230.8 |
| | EKEKEKGEKPRSS[CFGG-Dap- | | | | |
| | IDRIGHQSGLGC]PSFRHGSPSS | | | | |
| | GAPPPS-NH₂ | | | | |
| 140 | HOOC-(CH₂)₁₈-CO-(γGlu)- | 167 | Thioacetal | 5426.2 | 5426.4 |
| | EKEKEKGEKPRSS[CFGKRIDRI | | | | |
| | GRYSGLGCJPSFRHGSPSSGAPP | | | | |
| | PS-NH₂ | | | | |

### Example 141

Example 141 is a polypeptide represented by the following description (SEQ ID NO: 168). BFA-EKEKEKGEKGRSS[CFGGKIDRIGHYSGLGC]PSFRHGGPSSGAPPPS-NH₂

### (Disulfide Linkage)

### BFA Means Bifurcated Fatty Acid

Below is a depiction of the structure of Example 141 using the standard single letter L-amino acid codes.

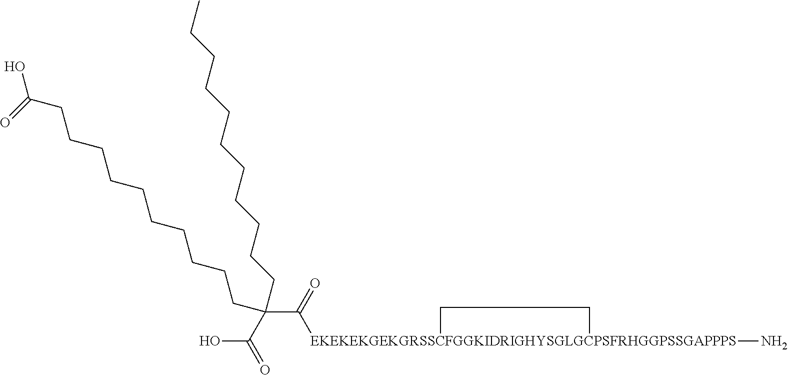

The peptide backbone of Example 141 was synthesized using Fluorenylmethyloxycarbonyl (Fmoc)/tert-Butyl (t-Bu) chemistry on a Symphony-X, 24-channel multiplex peptide synthesizer (Gyros Protein Technologies, Inc.). The solid support used consists of low loading 4-(2′,4′-Dimethoxyphenyl-Fmoc-aminomethyl)-phenoxyacetamido-norleucyl-4-Methylbenzhydrylamine resin (Fmoc-Rink-MBHA Low Loading resin, EMD Millipore), (100-200 mesh) with a 1% DVB cross-linked polystyrene core and a substitution range of 0.3-0.4 meq/g. Standard sidechain protecting groups were used for all Fmoc-L-Amino Acids used. Fmoc deprotection prior to each coupling step was done by treatment with 20% Piperidine in DMF, (1×4 minutes and 1×10 minutes 7) with nitrogen mixing followed by 6×DMF washing cycles. All amino acid couplings were performed for 1 hour using the Fmoc Amino Acid (0.3 M in DMF), N, N. N′,N′-Tetramethyl-O-(1H-benzotriazol-1-yl)uronium hexafluorophosphate (HBTU, Ambeed Inc; 0.9 M in DMF) and N,N-Diisopropylethylamine (DIPEA; 1.2 M in DMF), at a 9-fold molar excess of AA/HBTU and a 12-fold molar excess of DIPEA over the theoretical resin loading level. After the primary sequence of the peptide was synthesized and the final Fmoc-deprotection and washes were completed.

Attachment of the fatty acid (BFA) moiety was accomplished by manual addition of a 2-3-fold excess of 13-tert-butoxy-2-tert-butoxycarbonyl-13-oxo-2-undecyl-tridecanoicacid which was dissolved in 5-7 mL of DMF and transferred to the Symphony-X reaction vessel followed by the addition of 2-3-fold excess diisopropylcarbodiimide (DIC) and 2-3 fold excess of ethyl-cyano(hydroxyamino)acetate (Oxyma). The reaction time for the BFA coupling was ˜18 hours, after which point the resin was washed 3× with DMF and a Kaiser test was performed to ensure coupling completion. After the acylation was completed, the peptidyl resin was transferred, as a DCM slurry, to disposable fritted plastic syringe fitted with Teflon stopcock and further washed with DCM were done, finally, the resin was thoroughly air-dried. The dry resin was then treated with 10 mL of cleavage cocktail consisting of trifluoroacetic acid (TFA), water, 3,6-dioxa-1,8-octanedithiol (DODT), triisopropylsilane (TIPS), (TFA:Water:DODT:TIPS: 92.5:2.5:2.5:2.5 v/v) for 2 hours at room temperature. After the 2 hr incubation, the resin was filtered off and collected in a 50 ml conical disposable tube containing 35 mL of cold diethyl ether (−20° C.) to precipitate the crude peptide. The peptide/ether suspension was then centrifuged at 4000 rpm for 2 min to form a solid pellet, the supernatant was decanted, and the solid pellet was triturated with ether two additional times and dried in vacuo.

### Disulfide Linkage Formation

The crude peptide was solubilized in a 50 mL falcon tube with ˜50 mL of a 10% acetonitrile solution in 0.1% TFA-H₂O. The solution was then added to an Erlenmeyer flask placed on a magnetic stirrer with requisite spin vane, diluted to 100 mL total volume with 0.1% TFA-H₂O (˜5 mg/mL crude peptide concentration) and then treated with several drops of a saturated Iodine in methanol solution until a faint yellow color persists. The reaction was stirred at RT for 10 minutes at which point the excess iodine was quenched with a few drops of 0.1 M aqueous ascorbic acid.

### HPLC Purification

The crude oxidation solution was then directly loaded onto a Waters semi-prep HPLC system and purified on a Symmetry C18 (7 μm, 19×300 mm; Waters) with linear gradients of 100% acetonitrile and 0.1% TFA/water buffer system (10-40% over 70 minutes). The purity of the peptide was assessed using analytical LC-MS and pooling criteria was >90%. The main pool of Example 141 was found to be >95.0%. Subsequent lyophilization of the final main product pool yields the lyophilized peptide as a TFA salt. The molecular weight was determined by analytical LC-MS (obsd:=5194.2; Calc=5194.8).

### Example 142

Example 142 is a polypeptide represented by the following description (SEQ ID NO:169). (BFAEN2-βAla)-EKEKEKG-PEG24-EK-βAla-RSS[CFGGKIDRIGHYSGLGC]PSFRHGGKSSGAPPPS-NH₂

### (Thioacetal Linkage)

BFAEN2 means Bifurcated Fatty Acid Enantiomer 2

Below is a depiction of the structure of Example 142 using the standard single letter amino code with the exception of βAla where the structure of the amino acid has been expanded.

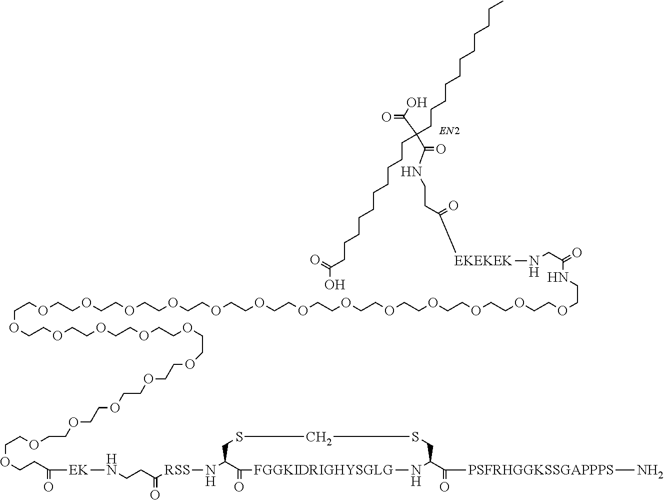

The peptide backbone of Example 142 was synthesized as described for Example 141. An exception to the synthetic method was the coupling of Fmoc-N-amido-PEG24-OH which required a pause in the automated synthesis protocol. The PEG24 residue coupling was accomplished by the manual addition of, a 3-fold excess of Fmoc-N-amido-PEG24-OH (BroadPharm) dissolved in 5 mL of DMF and transferred to the Symphony-X reaction vessel followed by the addition of 2-fold excess of diisopropylcarbodiimide (DIC) and 2-fold excess of ethyl-cyano(hydroxyamino)acetate (Oxyma). The reaction time for the coupling was ˜18 hours, after which point the resin was washed 3× with DMF and a Kaiser test was performed to ensure coupling completion. The automated methods were resumed to complete the synthesis of the rest of sequence and the fatty acid was coupled as described below.

### Attachment of BFAEN2-βAla

Attachment of the fatty acid was accomplished by manual addition of a 1.5 fold excess of 2-[[3-(2,5-dioxypyrrolidin-1-yl)oxy-3-oxo-propyl]carbamoyl]-2-undecyl-tridecanedioic acid and 3-fold excess of N,N-Diisopropylethylamine (DIPEA) which were dissolved in 5-7 mL of DMF and transferred to the Symphony-X reaction vessel. The reaction time for the FA coupling was ˜18 hours, after which point the resin was washed 3× with DMF. The cleavage was then performed as described for Example 141, followed by disulfide linkage formation as described in Example 141.

### Thioacetal Linkage Formation

The crude oxidation solution was then directly loaded onto a Waters semi-prep HPLC and purified on a Symmetry C18 (7 μm, 19×300 mm; Waters) with linear gradients of 100% acetonitrile and 0.1% TFA/water buffer system (10-40% over 70 minutes). The purity of the fractions was assessed using LC-MS and pooling criteria is >80% for fractions to be used for the thioacetal conversion. These fractions are combined and diluted 1:1 with a 50% mixture of acetonitrile-H₂O. The disulfide linkage was first reduced by addition of 1 mL of a 0.25M aqueous solution of Tris(2-carboxyethyl)phosphine (TCEP, TCI America) and then 400-500 uL of neat triethylamine (TEA, Sigma Aldrich) is added to bring the pH of solution to >7. After 10 minutes, 50-100 uL of diiodomethane (TCI America) was added followed by an additional 50-100 uL of TEA. The reaction is monitored by LC-MS and conversion is completed withing ˜18 hours.

The crude thioacetal solution is diluted 1:1 with H₂O and then loaded onto a Waters semi-prep HPLC system and purified on a Symmetry C18 (7 μm, 19×300 mm; Waters) with linear gradients of 100% acetonitrile and 0.1% formic acid/water buffer system (5-35% over 70 minutes). The purity of the peptide is assessed using LC-MS and pooling criteria is >90% and ˜100 uL of neat TFA is added to the pooled fractions. The main pool of Example 142 was found to be >95.0%. Subsequent lyophilization of the final main product pool yields the lyophilized peptide TFA salt. The molecular weight was determined by analytical LC-MS (obsd:=6452.5; Calc=6453.4).

### Example 143

Example 143 is a polypeptide represented by the following description (SEQ ID NO:170) (BFAEN2-βAla)-EKEKEKG-PEG24-E-4Pal-KRSS[CFGKKIDRIGHYSGLGC]PSFRHGGKSSGAPPPS-NH₂

### (Thioacetal Linkage)

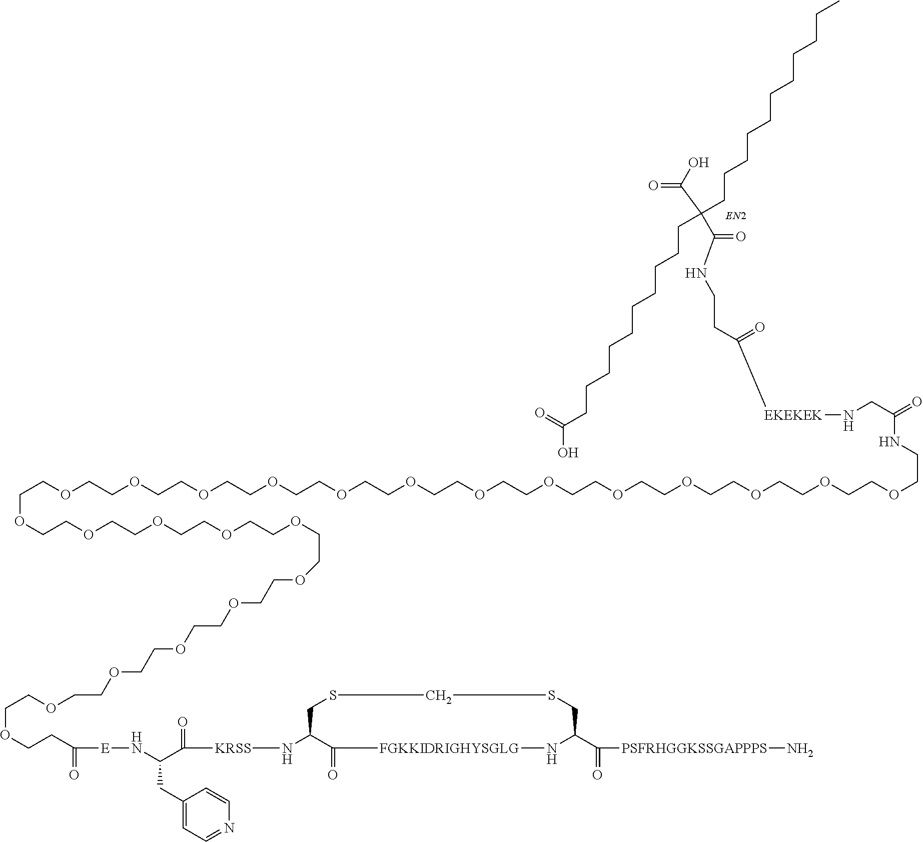

Example 143 was synthesized substantially as described in Example 142. The molecular weight was determined by LC-MS (obsd:=6601.6; Calc=6601.5).

### Example 144

Example 144 is a polypeptide represented by the following description (SEQ ID NO:171) (BFAEN2-βAla)-EKEKEKG-PEG24-EK-βAla-RSS[CFGKRIDRIGHQSGLGC]PSFRHGSPSSGAPPPS-NH₂

### (Thioacetal Linkage)

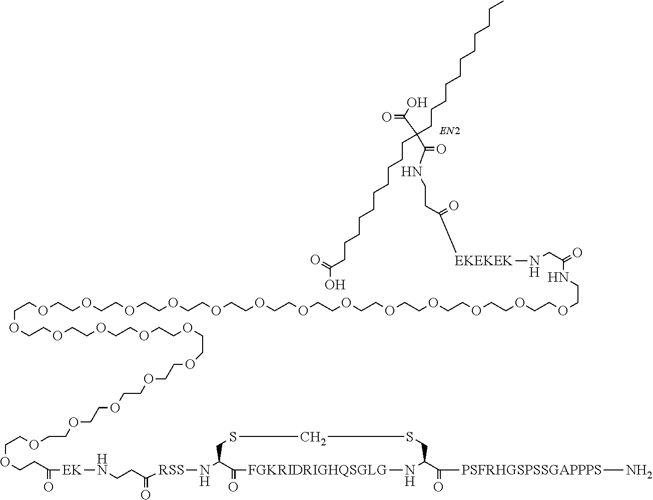

Example 144 was synthesized substantially as described for Example 142. The molecular weight was determined by LC-MS (obsd:=6515.6; Calc=6516.4).

### Example 145

Example 145 is a polypeptide represented by the following description (SEQ ID NO: 172) (BFAEN2-γGlu)-KEKEKG-PEG24-EK-βAla-RSS[CFGGKIDRIGHYSGLGC]PSFRHGSPSSGAPPPS-NH₂

### (Thioacetal Linkage)

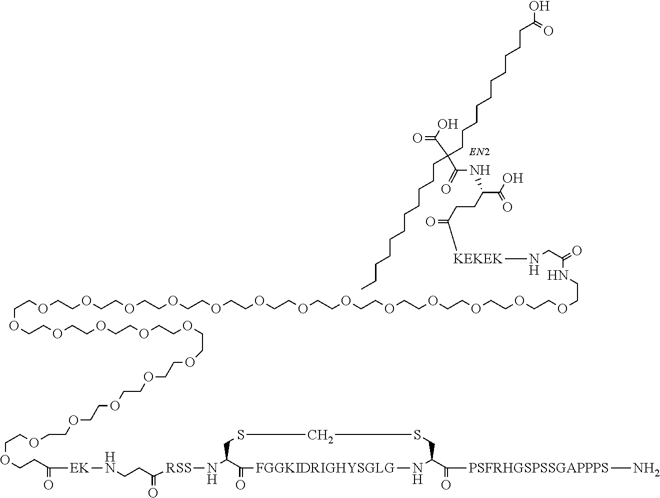

Example 145 was synthesized substantially as described for Example 142 with the attachment of the BFAEN2-γGlu as described below.

### Attachment of BFAEN2-γGlu

Attachment of the fatty acid was accomplished by manual addition of 1.5 fold excess of 2-[[(1S)-1-tert-butoxycarbonyl-4-(2,5-dioxopyrrolidin-1-yl)oxy-4-oxo-butyl]carbamoyl]-2-undecyl-tridecanedioic acid and 3-fold excess of N,N-Diisopropylethylamine (DIPEA) which were dissolved in 5-7 mL of DMF and transferred to the Symphony-X reaction vessel. The reaction time for the FA coupling was ˜18 hours, after which point the resin was washed 3× with DMF. The cleavage was then performed as described for Example 141.

Disulfide linkage formation, followed by thioaetal linkage formation was performed as described in Example 142.

The molecular weight was determined by LC-MS (obsd:=6381.6; Calc=6381.2).

### In Vitro Function

### Functional Activity Assays:

Functional activity of the ANP polypeptides is determined in NPR-A-expressing HEK-293 clonal cell lines as explained below.

Full-Length cDNA Cloning and Generation of Cell Lines Overexpressing Natriuretic Peptide Receptors (NPRs)

All sequences were verified by full-length sequencing performed by ACGT DNA Sequencing Services (Wheeling, IL). The target cDNA was cloned into pJTI R4 CMV-TO MCS pA vector and then co-transfected with pJT1R4 Int vector into Jump-in™ T-Rex™ HEK293 cells for mammalian inducible expression using Jump-in™ T-Rex™ HEK293 kit and Lipofectamine LTX and Plus Reagent following manufacturer's protocols, as briefly described below.

Jump-in™ T-Rex™ HEK293 cells were plated in a BioCOAT® poly-D-lysine coated 6-well plate (Becton Dickinson, cat no. 354413) at 1 million cells/well in 2 mL culture medium and incubated for 18 h at 37° C. and 5% CO₂ to 50-70% confluence. A cDNA mix was made in a 50 mL tube by adding 1.5 μg target cDNA, 1.5 μg pJT1R4 Int vector, 3 μL Plus Reagent, and 300 μL Opti-MEM I sequentially into the tube. A reagent mix was made in a separate 50 mL tube by adding 7.5 μL Lipofectamine LTX into 300 μL Opti-MEM I. The mixtures were incubated for 5 min at room temperature. The cDNA mix was then transferred into the reagent mix, mixed well, and incubated for additional 30 min at room temperature. A 500 μL of cDNA/Lipofectamine complex was then transferred to the wells of the cell plate in which the culture medium was changed to 2 mL of transfection medium containing DMEM with 4.5 g/L D-glucose supplemented with 10% FBS-HI and 20 mM HEPES. Transfected cells were cultured for 48 h in an incubator at 37° C. and 5% CO₂. A subclone or pool from each overexpressing cell line was maintained in culture medium with the addition of 2 mg/mL G418 sulfate for the clone selection based on its built-in resistance to G418 sulfate for at least 3 weeks with medium changed every 2-3 days.

NPRs were overexpressed in T-Rex™ HEK293 cells following the induction with 300 ng/mL tetracycline in culture medium for 48 h. Induced cell lines in an exponential growth phase were treated with 0.05% trypsin-EDTA for a few seconds at room temperature, harvested in cell medium containing FBS to neutralize the trypsin, counted, and cryopreserved at the density of 2 million cells/mL in cell preservation solution containing FBS-HI with 5% DMSO. Cryopreserved cells were stored at −80° C. for a few days prior to transferring to a liquid nitrogen tank. Induced cell lines were then used for suspension assays to measure the activity of polypeptides to stimulate cGMP production in cGMP assays or for the preparation of cell membranes to measure the binding activity of polypeptides in competitive radioligand binding assays, as described below.

Human and Rat NPRA cGMP Activity Assays

Cells overexpressing human or rat NPRA were plated in 96-well assay plates and stimulated in the presence of assay buffer (normalized as 0% response), human ANP, amidated rat ANP (100 nM, normalized as 100% response), or varying concentrations of test polypeptides. Test polypeptides were added starting at 10 μM concentration and at 10-fold decreasing concentrations to obtain 8-point concentration-response curves (i.e., 10 μM to 1 pM). The quantity of cGMP generated was detected using HTRF® technology and normalized to maximum amount produced by 100 nM amidated rat ANP and the minimum amount produced by assay buffer. Detailed steps are outlined below.

Stock solutions of test polypeptides (2 mM) dissolved in DMSO were first diluted 100-fold in assay buffer containing HBSS with Ca²⁺ and Mg²⁺, 5 mM HEPES, 0.5 mM IBMX, and 0.1% BSA or 0.1% casein (pH 7.4). The polypeptides were further serially diluted in 1:10 dilution steps in assay buffer containing 0.1% BSA or 0.1% casein to generate 8-point 2× working stock solutions ranging from 20 μM to 2 μM.

A 10 μL cell suspension containing 4000 cells was plated in Costar® half-area white opaque 96-well plates (Corning, cat no. 3693). Then 10 μL of assay buffer (basal activity), 200 nM of amidated rat ANP (maximum activity) or 2× working stock solutions of test polypeptides were transferred into the plate. Final concentration of DMSO in each well was 0.5%. The plate was shaken for 15 sec and then incubated for 40 min at room temperature.

The cGMP generated was measured using cGMP kit following the manufacturer's directions, as described below.

Cyclic GMP (cGMP) standards provided in the kit were serially diluted 1:3 ranging from 1 μM to 0.17 nM in assay buffer containing 0.1% BSA plus 0.5% DMSO or 0.1% casein plus 0.5% DMSO. The cGMP standards (20 μL) were then transferred to a separate Costar® 3693 plate.

The cGMP production was terminated, and the cGMP content was measured by sequentially adding 10 μL of cGMP-d2 and 10 μL of anti-cGMP-Cryptate, which were previously diluted 1:50 in lysis buffer provided in the kit. The plate was shaken for 15 sec, incubated for 2 h at room temperature in dark, and read in a Pherastar® FSX plate reader (BMG LABTECH, Ortenberg, Germany) at 337 nm for excitation and 665 nm/620 nm for emission.

The ratios of 665 nm/620 nm multiplied by 10000 were plotted with log-scale cGMP standard concentrations to generate a standard curve using an internally created 4-parameter nonlinear regression curve fitting template. The quantity of cGMP produced by cells overexpressing NPRA was interpolated using the cGMP standard curve. A 100% response was determined from wells in the presence of a saturating concentration of amidated rat ANP (100 nM). A 0% response was determined from wells containing assay buffer. The 8-point concentration-response curve for test polypeptides (10 μM to 1 μM) was fitted to a 4-parameter model using Prism 9 (GraphPad Software, Inc., San Diego, CA) to determine potency (EC₅₀) values and maximal activation (% Max).

Data for exemplary analogs and hANP are shown in Table 2 below.

TABLE 2
Functional cGMP Potency (EC₅₀) for exemplary ANP
polypeptides and Comparator polypeptide hANP. EC₅₀
is the concentration of polypeptide causing half-
maximal simulation in a dose response curve.

| | | cGMP Assay | | cGMP Assay | |
|---|---|---|---|---|---|
| | SEQ | (EC₅₀, pM) w/BSA | | (EC₅₀, pM) w/Casein | |
| Ex | ID NO | hNPRA | rNPRA | hNPRA | rNPRA |
| human ANP | 1 | 205 | 315 | 177 | 279 |
| rat ANP | 173 | 138 | 114 | 128 | 73 |
| (amidated) | | | | | |
| 1 | 45 | 11617 | 81882 | 335 | 1810 |
| 2 | 60 | 93424 | 1272591 | 1016 | 14104 |
| 3 | 107 | 6918 | 958800 | 980 | 36550 |
| 4 | 144 | 6185 | 518700 | 813 | 22525 |
| 5 | 146 | 33887 | 276367 | 679 | 1132 |
| 6 | 158 | 33292 | 596480 | 1532 | 30482 |
| 7 | 159 | 69562 | 1166520 | 5668 | 184378 |
| 8 | 28 | 81280 | 38048 | 1496 | 493 |
| 9 | 29 | 100500 | 46740 | 1874 | 509.4 |
| 10 | 30 | 64190 | 88400 | 1308 | 501 |
| 11 | 31 | 24435 | 24690 | 1020 | 242 |
| 12 | 32 | 108700 | 85470 | 1517 | 670 |
| 13 | 33 | 145200 | 24000 | 1919 | 241 |
| 14 | 34 | 27740 | 37810 | 685 | 1172 |
| 15 | 35 | 57270 | 32560 | 912 | 254 |
| 16 | 36 | 54690 | 480450 | 807 | 4068 |
| 17 | 37 | 153100 | 360700 | 20500 | 9044 |
| 18 | 38 | 17090 | 43200 | 460 | 693 |
| 19 | 39 | 67250 | 258900 | 852 | 1830 |
| 20 | 40 | 129100 | 104185 | 2902 | 1391 |
| 21 | 41 | 27780 | 268400 | 824 | 1950 |
| 22 | 42 | 25060 | 191900 | 840 | 1688 |
| 23 | 43 | 125100 | 218900 | 3491 | 1681 |
| 24 | 44 | 20980 | 66080 | 560 | 702.65 |
| 25 | 46 | 12030 | 57300 | 436 | 654 |
| 26 | 47 | 17770 | 119200 | 682 | 803 |
| 27 | 48 | 18760 | 130000 | 130 | 807 |
| 28 | 49 | 11270 | 92910 | 131 | 1028 |
| 29 | 50 | 32162 | 1906500 | 482 | 16842 |
| 30 | 51 | 16670 | 123000 | 302 | 1128 |
| 31 | 52 | 13845 | 795250 | 332 | 12216 |
| 32 | 53 | 27400 | 358600 | 246 | 1434 |
| 33 | 54 | 22230 | 112000 | 199 | 383 |
| 34 | 55 | 26090 | 421600 | 291 | 1202 |
| 35 | 56 | 12200 | 178600 | 118 | 427 |
| 36 | 57 | 18000 | 86480 | 118 | 277 |
| 37 | 58 | 14400 | 84550 | 196 | 461 |
| 38 | 59 | 16040 | 40528 | 317 | 373 |
| 39 | 61 | 9968 | 93270 | 276 | 439 |
| 40 | 62 | 23025 | 294700 | 351 | 2040 |
| 41 | 63 | 29140 | 239100 | 224 | 1136 |
| 42 | 64 | 24980 | 69030 | 358 | 533 |
| 43 | 65 | 27616 | 76275 | 301 | 605 |
| 44 | 66 | 33107 | 93038 | 431 | 1346 |
| 45 | 67 | 25210 | 84210 | 362 | 510 |
| 46 | 68 | 21250 | 88260 | 230 | 460 |
| 47 | 69 | 24340 | 119500 | 299 | 650 |
| 48 | 70 | 50470 | 91800 | 611 | 755 |
| 49 | 71 | 29940 | 87000 | 299 | 667 |
| 50 | 72 | 22510 | 143850 | 614 | 1929 |
| 51 | 73 | 11110 | 82660 | 361 | 909 |
| 52 | 74 | 24710 | 823400 | 293 | 10950 |
| 53 | 75 | 14870 | 779700 | 484 | 7118 |
| 54 | 76 | 19275 | 77200 | 369 | 796 |
| 55 | 77 | 18450 | 34580 | 451 | 493 |
| 56 | 78 | 145000 | 1376000 | 855 | 11250 |
| 57 | 79 | 48840 | 357200 | 1157 | 9505 |
| 58 | 80 | 103500 | 1378000 | 1218 | 7151 |
| 59 | 81 | 25340 | 381100 | 622 | 2495 |
| 60 | 82 | 81660 | 962300 | 453 | 4827 |
| 61 | 83 | 9002 | 462700 | 912 | 31950 |
| 62 | 84 | 17330 | 588400 | 2164 | 25520 |
| 63 | 85 | 63802 | 784650 | 1090 | 10607 |
| 64 | 86 | 24500 | 366100 | 758 | 3402 |
| 65 | 87 | 81970 | 893300 | 614 | 7415 |
| 66 | 88 | 140300 | 1747000 | 826 | 9770 |
| 67 | 89 | 138833 | 1554000 | 1218 | 18773 |
| 68 | 90 | 96830 | 1284000 | 924 | 12110 |
| 69 | 91 | 61450 | 804500 | 801 | 20150 |
| 70 | 92 | 92520 | 711900 | 1836 | 11770 |
| 71 | 93 | 15629 | 594165 | 594 | 21018 |
| 72 | 94 | 46448 | 1065885 | 588 | 40544 |
| 73 | 95 | 5822 | 76416 | 1233 | 26019 |
| 74 | 96 | 171400 | 1133000 | 1588 | 5816 |
| 75 | 97 | 16950 | 532600 | 1059 | 23795 |
| 76 | 98 | 79940 | 607000 | 2618 | 8278 |
| 77 | 99 | 46200 | 637400 | 1274 | 15720 |
| 78 | 100 | 19740 | 626900 | 2389 | 33370 |
| 79 | 101 | 4581 | 261100 | 273 | 3437 |
| 80 | 102 | 4750 | 364050 | 234 | 3550 |
| 81 | 103 | 5714 | 126883 | 388 | 1914 |
| 82 | 104 | 32610 | 835700 | 623 | 25400 |
| 83 | 105 | 38405 | 860150 | 1043 | 16680 |
| 84 | 106 | 9727 | 546900 | 1011 | 24610 |
| 85 | 108 | 13200 | 418900 | 361 | 7785 |
| 86 | 109 | 25190 | 867700 | 1021 | 17940 |
| 87 | 110 | 3375 | 463100 | 621 | 8866 |
| 88 | 111 | 16280 | 1048000 | 520 | 11450 |
| 89 | 112 | 10790 | 819000 | 806 | 13800 |
| 90 | 113 | 19720 | 734300 | 452 | 13230 |
| 91 | 114 | 13860 | 549000 | 1003 | 16170 |
| 92 | 115 | 10110 | 289700 | 772 | 12380 |
| 93 | 116 | 53160 | 702300 | 1653 | 51990 |
| 94 | 117 | 73900 | 1299000 | 1604 | 56960 |
| 95 | 118 | 7026 | 321800 | 771 | 12296 |
| 96 | 119 | 12715 | 400650 | 720 | 12168 |
| 97 | 120 | 7870 | 198300 | 720 | 9146 |
| 98 | 121 | 40865 | 625250 | 1842 | 17890 |
| 99 | 122 | 13600 | 455100 | 800 | 6290 |
| 100 | 123 | 6584 | 272900 | 944 | 10910 |
| 101 | 124 | 11590 | 138200 | 282 | 1340 |
| 102 | 125 | 3492 | 154100 | 749 | 15090 |
| 103 | 126 | 12320 | 375700 | 567 | 2138 |
| 104 | 127 | 4473 | 311500 | 527 | 2632 |
| 105 | 128 | 9179 | 135300 | 461 | 4383 |
| 106 | 129 | 4332 | 186800 | 741 | 9631 |
| 107 | 130 | 3773 | 149100 | 646 | 3775 |
| 108 | 131 | 6141 | 193050 | 497 | 11224 |
| 109 | 132 | 2558 | 276400 | 488 | 16840 |
| 110 | 133 | 2207 | 89140 | 580 | 5318 |
| 111 | 134 | 31730 | 568300 | 1532 | 13430 |
| 112 | 135 | 7471 | 93460 | 376 | 1541 |
| 113 | 136 | 6021.5 | 149150 | 621 | 6356 |
| 114 | 137 | 10440 | 72210 | 976 | 1206 |
| 115 | 138 | 2830 | 100000 | 726 | 5646 |
| 116 | 139 | 8969 | 205400 | 745 | 3832 |
| 117 | 140 | 9174 | 199800 | 922 | 6041 |
| 118 | 141 | 31120 | 681700 | 3323 | 55700 |
| 119 | 142 | 12820 | 623400 | 493 | 17930 |
| 120 | 143 | 10000 | 695800 | 542 | 25430 |
| 121 | 145 | 35290 | 368120 | 2573 | 29800 |
| 122 | 147 | 6380 | 149800 | 490 | 3772 |
| 123 | 148 | 9546 | 147300 | 902 | 3990 |
| 124 | 149 | 3982 | 74302 | 656 | 4508 |
| 125 | 150 | 5169 | 80170 | 736 | 4851 |
| 126 | 151 | 6372 | 88530 | 1093 | 3756 |
| 127 | 152 | 41490 | 341300 | 703.2 | 2015 |
| 128 | 153 | 23670 | 498000 | 566 | 2013 |
| 129 | 154 | 2957 | 59410 | 1006. | 2277 |
| 130 | 155 | 1273 | 197400 | 669.1 | 9165 |
| 131 | 156 | 37560 | 815850 | 392 | 2159 |
| 132 | 157 | 5304 | 259533 | 704 | 9386 |
| 133 | 160 | 302200 | 1447000 | 38680 | 580500 |
| 134 | 161 | 84730 | 2253000 | 930 | 191900 |
| 135 | 162 | 104100 | 6517000 | 497 | 185900 |
| 136 | 163 | 100100 | 2428000 | 1379 | 32890 |
| 137 | 164 | 155300 | 2189000 | 769 | 76860 |
| 138 | 165 | 1410000 | 3796000 | 5349 | 71710 |
| 139 | 166 | 2870000 | 1729000 | 19700 | 200000 |
| 140 | 167 | 11690 | 4178000 | 692 | 41100 |

As seen in Table 2, in the presence of BSA, exemplary ANP polypeptides have agonist activities as determined by hNPR-A assays, which are lower than the native ligand hANP. However, when the assays are conducted in the presence of casein (instead of serum albumin) as a nonspecific blocker, which does not interact with the fatty acid moieties of the analyzed molecules, the exemplary ANP polypeptides have agonist activities which are comparable to hANP.

Human and Rat NPRB cGMP Activity Assays

Functional activity of the ANP polypeptides is determined in NPR-B-expressing HEK-293 clonal cell lines as explained below.

Cells overexpressing human or rat NPRB were plated in 96-well assay plates and stimulated in the presence of assay buffer (normalized as 0% response), human CNP-22 (1 μM, normalized as 100% response), or varying concentrations of test polypeptides. Test polypeptides were added starting at 10 μM concentration and at 10-fold decreasing concentrations to obtain 10-point concentration-response curves (i.e., 10 μM to 0.01 μM). The quantity of cGMP generated was detected using HTRF® technology and normalized to the maximum amount produced by 1 μM human CNP-22 and the minimum amount produced by assay buffer. Detailed steps are outlined below.

Stock solutions of test polypeptides (2 mM) dissolved in DMSO were first diluted 100-fold in assay buffer containing HBSS with Ca²⁺ and Mg²⁺, 5 mM HEPES, 0.5 mM IBMX, and 0.1% BSA or 0.1% casein (pH 7.4). The polypeptides were further serially diluted in 1:10 dilution steps in assay buffer containing 0.1% BSA or 0.1% casein to generate 10-point 2× working stock solutions ranging from 20 μM to 0.02 μM.

A 15 μL assay buffer (basal activity), 1 μM human CNP-22 (maximum activity) or 2× working stock solutions of test polypeptides were transferred into a Costar® 3693 plate. A 15 μL cell suspension containing 4000 cells was then plated. Final concentration of DMSO in each well was 0.5%. The plate was shaken for 15 sec and then incubated for 40 min at room temperature.

The cGMP generated was measured using cGMP kit following the manufacturer's directions as described below.

Cyclic GMP (cGMP) standards provided in the kit were serially diluted 1:3 ranging from 1 μM to 0.17 nM in assay buffer containing 0.1% BSA plus 0.5% DMSO or 0.1% casein plus 0.5% DMSO. The cGMP standards (30 μL) were transferred to a separate Costar® 3693 plate.

The cGMP production was terminated, and the cGMP content was measured by sequentially adding 15 μL of cGMP-d2 and 15 μL of anti-cGMP-Cryptate which were previously diluted 1:50 in lysis buffer provided in the kit. The plate was shaken for 15 sec, incubated for 2 h at room temperature in dark, and read in a Pherastar® FSX plate reader at 337 nm for excitation and 665 nm/620 nm for emission.

The ratios of 665 nm/620 nm multiplied by 10000 were plotted with log-scale cGMP standard concentrations to generate a standard curve using an internally created 4-parameter nonlinear regression curve fitting template. The quantity of cGMP produced by cells overexpressing NPRB was interpolated using the cGMP standard curve. A 100% response was determined from wells in the absence of test polypeptide and the presence of a saturating concentration of human CNP-22 (1 μM). A 0% response was determined from wells containing assay buffer. The 10-point concentration-response curve for test polypeptides (10 μM to 0.01 μM) was fitted to a 4-parameter model using Prism 9 to determine potency (EC₅₀) values and maximal activation (% Max).

Similar to hANP, none of the exemplary polypeptides exhibited significant agonist activity at NPR-B.

### In Vivo Studies

### Pharmacokinetics in Male Sprague Dawley Rats:

The pharmacokinetics of the exemplary analogs are evaluated following a single subcutaneous administration of 200 nM/kg to male Sprague Dawley rats. Blood samples are collected over 120 hours, and resulting individual plasma concentrations are used to calculate pharmacokinetic parameters. Peptide plasma (K₃ EDTA) concentrations are determined using a qualified LC/MS method that measured the intact mass of the ANP polypeptide. Each peptide and an analog as an internal standard are extracted from 100% specie specified plasma using methanol with 0.1% formic acid. A Thermo Q-Exactive, High Resolution Instrument, and a Thermo Easy Spray PepMap are combined for LC/MS detection. Mean pharmacokinetic parameters are shown in Table 3.

TABLE 3
Mean Pharmacokinetic Parameters of Peptides Following
a Single Subcutaneous Administration of 200
nMol/kg to Male Sprague Dawley Rats.

| | | CL/F | t½ |
|---|---|---|---|
| Ex | SEQ ID NO | mL/kg/Hours | hours |
| 1 | 45 | 20.6 | 11 |
| 4 | 144 | 4.3 | 15 |
| 6 | 158 | 6.5 | 17 |
| 7 | 159 | 3.3 | 19 |
| 8 | 28 | 41.9 | 15 |
| 29 | 50 | 32.7 | 12 |
| 30 | 51 | 12.6 | 16 |
| 56 | 78 | 11.2 | 36 |
| 61 | 83 | 64.9 | 6 |
| 62 | 84 | 23.7 | 10 |
| 75 | 97 | 10.9 | 17 |
| 76 | 98 | 7.1 | 30 |

Studies in the Salty drinking water/Unilateral Nephrectomy/Aldosterone (SAUNA) Mouse Model

The effect of Exemplary ANP polypeptides is investigated in the Salty drinking water/Unilateral Nephrectomy/Aldosterone (SAUNA) Mouse Model, murine model of heart failure induced by chronic aldosterone infusion. After acclimation for approximately 2 weeks, heart failure is induced in male C57BL/6N (Taconic) mice by uninephrectomy, continuous d-aldosterone infusion and 1.0% sodium chloride in drinking water for 4 weeks (Tanaka et al., 2016; Valero-Munoz, Li, Wilson, Boldbaatar, et al., 2016; Valero-Munoz, Li, Wilson, Hulsmans, et al., 2016; Yang, Kong, Shuai, Zhang, & Huang, 2020; Yoon et al., 2021). Approximately two weeks after induction of the heart failure protocol, mice are distributed into groups to provide comparable variance in body weight and blood pressure (measured in conscious mice with a noninvasive tail cuff system (Kent Scientific); mice are randomized using Block Randomized Allocation Tool (BRAT, Eli Lilly and Company). Once randomized, mice are treated once daily via subcutaneous (SC) injection of an ANP polypeptide (0.4 mg/kg). Blood pressure is monitored weekly for the duration of the study. Two weeks after initiation of treatment (4 weeks post induction of heart failure) mice are anesthetized with isoflurane, intubated via tracheotomy and chest opened to expose the heart and allow placement of a pressure volume catheter (Transonic). The pressure volume (PV) catheter is introduced into the left ventricle via apical stab with a 27G needle. Calibration of the PV catheter is performed according to the manufacturer's instructions. Data are analyzed with LabChart pro software (AD Instruments). After PV loop measurements, mice are sacrificed, and ratio of heart weight to tibia length is used for indicator of hypertrophy.

The effect of Example 8 was investigated using the SAUNA Mouse Model described above. Administration of Example 8 resulted in decrease in blood pressure, heart weight and tibia length, and reduced left ventricle diastolic pressure.

In Vivo Monkey Studies—cGMP Levels

In vivo monkey studies were conducted as described in detail below. Young adult-to-adult male cynomolgus monkeys were given a single dose subcutaneously (SC). Blood was collected predose and at various pre-determined timepoints throughout the study period. Aliquot of cynomolgus monkey plasma was received for cyclic cGMP (cGMP) measurement and stored at −80° C. prior to use.

### Control Plasma

Control plasma was made for determining the recovery rate of spike-in cGMP using Enzo cGMP complete ELISA kit (Enzo Life Sciences, Inc, Farmingdale, NY, cat no. ADI-901-164). Briefly, blood from male Sprague Dawley rats (Inotiv, Indianapolis, IN), about 7 months old, was collected in a BD Vacutainer EDTA tube (Becton Dickinson, Franklin Lakes, NJ, cat no. BD-367856) with volume of approximate 4 mL through retro orbital bleeding. Plasma was prepared by spinning the tube in an Eppendorf Refrigerated Centrifuge 5810R (Brinkman Instruments, Inc, Westbury, NY) at 3500 rpm (3000×g) for 10 min at 4° C. Plasma was collected as Positive Control plasma. Cyclic GMP (cGMP standard from Enzo cGMP complete ELISA kit) was added to the positive control plasma at a final concentration of additional 40 nM. Positive Control and Spiked-in Control plasma were aliquoted and stored at −80° C.

Assay Method—Monkey Plasma cGMP ELISA

The cGMP content was measured using the Enzo cGMP complete ELISA kit following the manufacturer's directions with modifications as described below. The cGMP standards provided in the kit were diluted 1:3 ranging from 50 nM to 0.023 nM in 1× assay buffer that was diluted in water from 2× assay buffer provided in the kit. The cGMP standards (150 L) were transferred to a 96-well polypropylene plate (Thermo Scientific, cat no. 442587).

Plasma samples were thawed from −80° C. and diluted 1:20 in 1× assay buffer in the above plate with a final volume of 150 μL. Assay buffer was added (150 μL) for both a non-specific binding control (in the absence of cGMP antibody) and a maximum binding control (in the absence of competing cGMP) in duplicate wells. Positive Control and Spiked-in Control plasma were diluted 1:20 in 1× assay buffer with a final volume of 150 μL in duplicate wells of the above plate. All diluted plasma was mixed by pipetting up and down several times.

Acetylation reagent mix was prepared by adding 1-part of acetic anhydride into 2-parts of triethylamine provided in the kit and mixing well using Vortex (Scientific Industries, Inc, Bohemia, NY). All controls, standards, and plasma samples were acetylated by adding 15 μL of acetylation reagent mix into the plate, one column at a time, and shaking for 1 min on a Titer Plate Shaker (Lab-Line Instrument, Inc., Melrose Park, IL) at room temperature. The plate was shaken for an additional 1 min following the acetylation of the last column to ensure the reaction was completed.

Acetylated controls, standard and plasma samples were transferred (100 μL) to an ELISA plate (n=1). Then 50 μL of cGMP conjugate were added to each well followed by the addition of 50 μL cGMP antibody to each well except for two non-specific binding wells, in which 50 μL of 1× assay buffer were added. The plate was then sealed and shaken for 2 h at room temperature on the plate shaker. Following this incubation, the plate was washed five times using 200 μL of 1× wash buffer diluted in water from 5× wash buffer provided in the kit. Then, 200 μL of para-Nitrophenylphosphate (pNpp) were added to each well. The plate was sealed with a new plate sealer and shaken in dark for 1 h at room temperature on the plate shaker. Finally, 50 μL of stop solution were added to each well to stop the enzyme reaction. The plate was then read at 405 nm using SpectraMax Plus (Molecular Devices, San Jose, CA).

### Replicates

N=3 monkeys per group.

### Data Analysis

The mean absorbance at 405 nm (O.D. 405 nm) for the non-specific binding controls was subtracted from the O.D. 405 nm of all samples. The subtracted O.D. 405 nm of all samples were then normalized with the mean subtracted O.D. 405 nm of the maximum binding controls as B/B₀%. B/B₀% of cGMP standards were then plotted with log scale cGMP standard concentrations to generate a standard curve using an internally created 4-parameter nonlinear regression curve fitting template. The quantity of total cGMP presented in the experimental samples was interpolated using this standard curve in the template. The net cGMP changes (nM) were calculated by subtracting cGMP value of each animal which was measured in the plasma prior to dosing the respective polypeptide as shown in Table 4 (predosing, Time 0) from cGMP value of the same animal at each time point postdose. Microsoft Excel (Microsoft, Redmond, WA) was used to graph cGMP (nK, mean±SEM) and net cGMP changes (nM, mean±SEM) at varying time points. Monkey cGMP Data is shown in Table 4.

TABLE 4

| Example | Dose | Net cGMP (nM) Change from Baseline | | | | | | |
|---|---|---|---|---|---|---|---|---|
| # | (nmol/kg) | 0.083 h | 0.25 h | 0.5 h | 1 h | 2 h | 4 h | 8 h |
| 1 | 100 | 2.21 | 9.18 | 18.73 | 27.34 | 37.39 | 34.82 | 22.24 |
| 2 | 30 | 1.39 | 4.06 | 4.07 | 4.19 | −0.22 | 0.08 | 3.28 |
| 2 | 100 | 2.8 | 12.56 | 11.31 | 8.62 | 0.5 | 2.54 | 6.66 |
| 2 | 300 | 6.7 | 33 | 43.45 | 37.82 | 32.01 | 24.72 | 37.43 |
| 2 | 1000 | 10.16 | 57.63 | 98.81 | 103.78 | 64 | 10.35 | 41.22 |
| 3 | 100 | 4.1 | 17.89 | 27.34 | 31.14 | 32.56 | 32.25 | 25.95 |
| 4 | 3 | −0.83 | −0.4 | −0.15 | −0.79 | −0.76 | −0.47 | 3.72 |
| 4 | 10 | NT | NT | NT | NT | 4.57 | 12.42 | 14.86 |
| 4 | 30 | NT | NT | NT | NT | 21.5 | 32.08 | 34.86 |
| 4 | 100 | NT | NT | NT | NT | 49.51 | 59.41 | 42.29 |
| 4 | 100 | 3.5 | 13.15 | 22.17 | 20.35 | 21.06 | 23.76 | 15.58 |
| 4 | 300 | NT | NT | NT | NT | 98.01 | 85.85 | 53.11 |
| 6 | 25 | −2.93 | 0.03 | 1.5 | 1.64 | 6.23 | 7.98 | 20.26 |
| 7 | 25 | −1.1 | −1.04 | 2.16 | 2.41 | 1.45 | 3.9 | 14.3 |
| 7 | 25 | 0.2 | 1.69 | 4.14 | 2.62 | 3.84 | 6.17 | 11.73 |
| 7 | 50 | −0.94 | 0.47 | 0.93 | 4.95 | 5.72 | 13.07 | 24.58 |
| 7 | 100 | 2.42 | 8.95 | 11.78 | 17.22 | 22.56 | 27.89 | 41.05 |
| 7 | 300 | −1 | 9.8 | 23.08 | 37.7 | 53.76 | 54.16 | 44.13 |
| 29 | 100 | 2.13 | 3.52 | 9.03 | 10.14 | 15.28 | 17.75 | 26.8 |
| 56 | 100 | −2.8 | −0.66 | −0.65 | −0.51 | −1.82 | 1.83 | 10.57 |
| 61 | 100 | 4.41 | 24.85 | 27.32 | 28.35 | 25.21 | 28.38 | 33.73 |
| 81 | 100 | 5.6 | 32.08 | 37.47 | 43.98 | 38.64 | 31.25 | 23.05 |
| 89 | 100 | 6.83 | 20.73 | 33.07 | 35.04 | 36.11 | 42.86 | 36.71 |
| 106 | 100 | 3.54 | 19.8 | 25.25 | 24.83 | 26.38 | 26.16 | 14.03 |
| 127 | 50 | −0.87 | 4.09 | 8.84 | 8.13 | 8.92 | 10.25 | 13.28 |
| 132 | 25 | 2.96 | 11.54 | 25 | 29.41 | 28.64 | 28.7 | 24.12 |
| 136 | 50 | 3.25 | 12.48 | 20.78 | 12.12 | 9.42 | 8.76 | 7.65 |
| 144 | 100 | 5.9 | 18.26 | 27.08 | 36.51 | 35.21 | 27.1 | 20.15 |
| 145 | 25 | −0.82 | 2.94 | 4.37 | 3.19 | 8.88 | 11.23 | 17.87 |

| Example | Net cGMP (nM) Change from Baseline | | | | | | | | |
|---|---|---|---|---|---|---|---|---|---|
| # | 12 h | 24 h | 36 h | 48 h | 72 h | 96 h | 120 h | 144 h | 168 h |
| 1 | 19.05 | 16.84 | 17.46 | 12.14 | 9.81 | 10.89 | 8.92 | 9.71 | 8.18 |
| 2 | 3.34 | 8.88 | NT | 6.46 | 6.21 | 4.47 | 4.89 | 6.49 | 5.95 |
| 2 | 6.31 | 10.19 | NT | 5.05 | 4.47 | 8.44 | 4.3 | 1.77 | 7.01 |
| 2 | 36.62 | 37.61 | NT | 24.8 | 19.18 | 23.07 | 17.55 | 15.69 | 18.7 |
| 2 | 31.77 | 38.08 | NT | 27.41 | 22.49 | 25.37 | 19.01 | 19.69 | 20.33 |
| 3 | 26.49 | 19.56 | 16.01 | 10.79 | 9.28 | 9.23 | 9.96 | 8.99 | 9.69 |
| 4 | 7.53 | 5.4 | 11.74 | 2.99 | 0.03 | −1.15 | −0.02 | 0.16 | −2.03 |
| 4 | 16 | 14.43 | 20.84 | 16.53 | 5.18 | 1.93 | 0.69 | −0.1 | 0.3 |
| 4 | 30.33 | 20.57 | 26.96 | 17.93 | 5.07 | 7.97 | 9.92 | 7.07 | 9.85 |
| 4 | 29.13 | 13.98 | 18.07 | 11.58 | 8.8 | 5.89 | 8.1 | 3.77 | 8.04 |
| 4 | 14.92 | 11.14 | 8.97 | 5.62 | 2.9 | 4.82 | 4.43 | 5.24 | 2.94 |
| 4 | 38.75 | 17.46 | 17.49 | 15.02 | 12.59 | 12.35 | 12.49 | 10.25 | 12.43 |
| 6 | 25.73 | 23.75 | 22.42 | 16.24 | 9.97 | 11.47 | 4.62 | 6.86 | 5.86 |
| 7 | 15.75 | 20.19 | 20.61 | 19.48 | 19.25 | 11.24 | 11.21 | 12.51 | 8.98 |
| 7 | 13.86 | 19.02 | NT | 13.22 | 15.29 | 9.34 | 11.44 | 6.92 | 6.98 |
| 7 | 23.51 | 29.66 | NT | 20.37 | 18.64 | 19.24 | 14.1 | 12.83 | 16.25 |
| 7 | 44.99 | 49.08 | NT | 35.54 | 30.5 | 25.93 | 22.6 | 22.79 | 25.47 |
| 7 | 37.1 | 42.53 | NT | 25.82 | 19.1 | 23.36 | 16.05 | 17.85 | 13.2 |
| 29 | 28.52 | 31.75 | 32.52 | 21.75 | 12.46 | 12.31 | 8.52 | 8.28 | 4.1 |
| 56 | 12.11 | 17.03 | 16.59 | 11.66 | 11.45 | 9.32 | 9.31 | 7.36 | 1.99 |
| 61 | 23.51 | 27.96 | 24.75 | 11.81 | 14.82 | 20.25 | 12.15 | 18 | 11.81 |
| 81 | 27.6 | 22.83 | 24.25 | 15.44 | 12.4 | 10.48 | 12.63 | 9.54 | 7 |
| 89 | 33.87 | 30.9 | 24.59 | 16.29 | 15.56 | 12.74 | 18.95 | 12.03 | 8.03 |
| 106 | 17.64 | 9.38 | 12.13 | 8.71 | 5.67 | 6.4 | 7.88 | 7.54 | 7.66 |
| 127 | 18.76 | 20.16 | 19.43 | 18.43 | 12.9 | 11.67 | 9.32 | 9.3 | 9.36 |
| 132 | 20.96 | 19.79 | 17.54 | 12.7 | 13.11 | 11.68 | 13.22 | 11.64 | 7.08 |
| 136 | 7.65 | 11.83 | 11.18 | 5.65 | 2.61 | 2.05 | 2.71 | 3.72 | 3.36 |
| 144 | 13.2 | 10.23 | 9.57 | 7.11 | 5.22 | 6.83 | 3.5 | 4.48 | 8.71 |
| 145 | 22.09 | 24.61 | 31.53 | 21.81 | 21 | 14.82 | 14.4 | 11.99 | 5.89 |

As can be seen from Table 4, administration of the polypeptides of Examples 1, 2, 3, 4, 6, 7, 29, 56, 61, 81, 89, 106, 127, 132, 136, 144 and 145, respectively, to monkeys resulted in increased net cGMP levels.

In Vivo Dog Studies—cGMP Levels

In vivo dog studies were conducted as described in detail below. Young adult-to-adult male purebred beagle dogs of Labcorp stock colony, maintained at Labcorp-Madison, were given a single dose subcutaneously (SC). Blood was collected predose and at various pre-determined timepoints throughout the study period. Aliquot of beagle dog plasma was received for cyclic cGMP (cGMP) measurement and stored at −80° C. prior to use.

### Control Plasma

Control plasma was made for determining the recovery rate of spike-in cGMP using Enzo cGMP complete ELISA kit (Enzo Life Sciences, Inc, Farmingdale, NY, cat no. ADI-901-164). Briefly, blood from male Sprague Dawley rats (Inotiv, Indianapolis, IN), about 7 months old, was collected in a BD Vacutainer EDTA tube (Becton Dickinson, Franklin Lakes, NJ, Cat #BD-367856) with volume of approximate 4 mL through retro orbital bleeding. Plasma was prepared by spinning the tube in an Eppendorf Refrigerated Centrifuge 5810R (Brinkman Instruments, Inc, Westbury, NY) at 3500 rpm (3000×g) for 10 min at 4° C. Plasma was collected as Positive Control plasma. Cyclic GMP (cGMP standard from Enzo cGMP complete ELISA kit) was added to the positive control plasma at a final concentration of additional 40 nM. Positive Control and Spiked-in Control plasma were aliquoted and stored at −80° C.

Assay Method—Dog Plasma cGMP ELISA

The cGMP content was measured using the Enzo cGMP complete ELISA kit following the manufacturer's directions with modifications as described below. The cGMP standards provided in the kit were diluted 1:3 ranging from 50 nM to 0.023 nM in 1× assay buffer that was diluted in water from 2× assay buffer provided in the kit. The cGMP standards (150 L) were transferred to a 96-well polypropylene plate (Thermo Scientific, cat no. 442587).

Plasma samples were thawed from −80° C. and diluted 1:20 and 1:40 in 1× assay buffer in the above plate with a final volume of 150 μL. Assay buffer was added (150 μL) for both a non-specific binding control (in the absence of cGMP antibody) and a maximum binding control (in the absence of competing cGMP) in duplicate wells. Positive Control and Spiked-in Control plasma were diluted 1:20 in 1× assay buffer with a final volume of 150 μL in duplicate wells of the above polypropylene plate. All diluted plasma was mixed by pipetting up and down several times.

Acetylation reagent mix was prepared by adding 1-part of acetic anhydride into 2-parts of triethylamine provided in the kit and mixing well using Vortex (Scientific Industries, Inc, Bohemia, NY). All controls, standards, and plasma samples were acetylated by adding 15 μL of acetylation reagent mix into the above polypropylene plate, 8 wells in one column at a time, and shaking for 1 min on a Titer Plate Shaker (Lab-Line Instrument, Inc., Melrose Park, IL) at room temperature. The plate was shaken for an additional 1 min following the acetylation of the last column to ensure the reaction was completed.

Acetylated controls, standard and plasma samples were transferred (100 μL) to an ELISA plate (n=1). Then 50 μL of cGMP conjugate were added to each well followed by the addition of 50 μL cGMP antibody to each well except for two non-specific binding wells, in which 50 μL of 1× assay buffer was added. The plate was then sealed and shaken for 2 h at room temperature on the plate shaker. Following this incubation, the plate was washed five times using 200 μL of 1× wash buffer diluted in water from 5× wash buffer provided in the kit. Then, 200 μL of para-Nitrophenylphosphate (pNpp) were added to each well. The plate was sealed with a new plate sealer and shaken in dark for 1 h at room temperature on the plate shaker. Finally, 50 μL of stop solution were added to each well to stop the enzyme reaction. The plate was then read at 405 nm using SpectraMax Plus (Molecular Devices, San Jose, CA).

### Replicates

N=3 dogs per group.

### Data Analysis

The mean absorbance at 405 nm (O.D. 405 nm) for the non-specific binding controls was subtracted from the O.D. 405 nm of all samples. The subtracted O.D. 405 nm of all samples were then normalized with the mean subtracted O.D. 405 nm of the maximum binding controls as B/B₀%. B/B₀% of cGMP standards were then plotted with log scale cGMP standard concentrations to generate a standard curve using an internally created 4-parameter nonlinear regression curve fitting template. The quantity of total cGMP presented in the experimental samples was interpolated using this standard curve in the template. The net cGMP changes (nM) were calculated by subtracting cGMP value of each animal which was measured in the plasma prior to dosing the respective polypeptide as shown in Table 5 (predosing, Time 0) from cGMP value of the same animal at each time point postdose. Microsoft Excel (Microsoft, Redmond, WA) was used to graph cGMP (nM, mean±SEM) and net cGMP changes (nM, mean±SEM) at varying time points. Dog cGMP Data is shown in Table 5.

TABLE 5

| Example | Dose | Net cGMP (nM) Change from Baseline | | | | | | |
|---|---|---|---|---|---|---|---|---|
| # | (nmol/kg) | 0.083 h | 0.25 h | 0.5 h | 1 h | 2 h | 4 h | 8 h |
| 2 | 60 | −5.29 | 1.61 | 40.41 | 101 | 29.72 | 60.64 | 68.79 |
| 4 | 50 | 3.08 | 11.23 | 25.02 | 58.72 | 73.34 | 122.98 | 157.34 |
| 7 | 50 | −4.03 | 0.2 | 28.04 | 77.61 | 66.51 | 124.76 | 173.62 |

| Example | Net cGMP (nM) Change from Baseline | | | | | | | | |
|---|---|---|---|---|---|---|---|---|---|
| # | 12 h | 24 h | 36 h | 48 h | 72 h | 96 h | 120 h | 144 h | 168 h |
| 2 | 83.51 | 90.36 | 80.86 | 80.82 | 64.85 | 58.58 | 47.41 | 43.47 | 19.04 |
| 4 | 147.25 | 111.73 | 81.07 | 74.31 | 75.82 | 64.23 | 80.48 | 77.03 | 65.74 |
| 7 | 173.41 | 132.42 | 95.64 | 98.09 | 80.96 | 77.23 | 92.7 | 103.94 | 70.12 |

As can be seen from Table 5, administration of the polypeptides of Examples 2, 4 and 7, respectively, to dogs resulted in increased net cGMP levels.

SEQUENCES
rANP (rat ANP)
SEQ ID NO: 1
SLRRSS[CFGGRIDRIGAQSGLGC]NSFRY
(disulfide linkage between C7 and C23)
hANP (human ANP)
SEQ ID NO: 2
SLRRSS[CFGGRMDRIGAQSGLGC]NSFRY
(disulfide linkage between C7 and C23)
Formula I
SEQ ID NO: 3
X₁X₂X₃RSSCFX₉X₁₀X₁₁IX₁₃RIG
X₁₇X₁₈SGLGCPSX₂₆RX₂₈X₂₉
wherein:
X1 is absent, S or E,
X2 is absent, L, K, 4-Pal, H or E,
X3 is absent, R, B-Ala, P, K, E or G,
X9 is G, 4-Pal or H,
X10 is G, K, R or Dap,
X11 is R, K, G or Dap,
X13 is D or G,
X17 is A, H, Dap, K, R or Orn,
X18 is Q, Y or 4-Pal,
X26 is F or L,
X28 is Y, H or 4-Pal, and
X29 is either absent, GGP or
selected from SEQ ID NO: 4-20,
and the C-terminal amino acid
is optionally amidated.
SEQ ID NO: 4
SGAPPPE
SEQ ID NO: 5
KITAKEDE
SEQ ID NO: 6
GPSSGAPPPE
SEQ ID NO: 7
GPSSGAPPPS
SEQ ID NO: 8
GGSSGAPPPS
SEQ ID NO: 9
GGPSSGAPPPS
SEQ ID NO: 10
KGPSSGAPPPS
SEQ ID NO: 11
GGKSSGAPPPS
SEQ ID NO: 12
GGPPS-Aib-KPPPK
SEQ ID NO: 13
GSPSSGAPPPS
SEQ ID NO: 14
RITAREDKQGYA
SEQ ID NO: 15
RITAREDKQGEA
SEQ ID NO: 16
GSPSSGAPPPS-PEG24-G
SEQ ID NO: 17
SGSPSSGAPPPSG
SEQ ID NO: 18
GGESSGEPPPSEE
SEQ ID NO: 19
GSGSPSSGAPPPSG
SEQ ID NO: 20
SGSPSSGAPPPSEEEG
Formula II
SEQ ID NO: 21
Fatty acid-Z1-Z2-Z3-X₁X₂X₃RSSCFX₉X₁₀X₁₁I
DRIGX₁₇X₁₈SGLGCX₂₄SX₂₆RX₂₈
wherein
Fatty acid is a C16-C26 fatty acid,
Z1 comprises an amino acid selected
from γGlu, E and β-Ala,
Z2 is either absent or comprises a four
to ten amino acid sequence comprising
amino acids independently selected from
E, K, G, P, A and S, and
Z3 is either absent or comprises a
polyethylene glycol or a
(2-[2-(2-amino-ethoxy)-ethoxy]-acetyl)
moiety,
and the C-terminal amino acid
is optionally amidated.
SEQ ID NO: 22
EKEKEKG
SEQ ID NO: 23
EPEPEPG
SEQ ID NO: 24
APPSG
SEQ ID NO: 25
KEKEKG
SEQ ID NO: 26
EKEKEKE
SEQ ID NO: 27
X₁X₂X₃RSSCFX₉X₁₀X₁₁IX₁₃RIGX₁₇
X₁₈SGLGCPSX₂₆RX₂₈X₂₉
wherein:
X1 is S or E,
X2 is K or 4-Pal,
X3 is R, B-Ala or K,
X9 is G,
X10 is G or K,
X11 is R or K,
X13 is D or G,
X17 is H,
X18 is Q or Y,
X26 is F,
X28 is H,
and
X29 is selected from
(SEQ ID NO: 9)
GGPSSGAPPPS,
(SEQ ID NO: 11)
GGKSSGAPPPS,
and
(SEQ ID NO: 13)
GSPSSGAPPPS,
and the C-terminal amino acid
is optionally amidated.
SEQ ID NO: 28-167
Examples 1-140 respectively as
listed in Table 1
SEQ ID NO: 168-172
Examples 141-145 respectively.
(amidated rat ANP)
SEQ ID NO: 173
SLRRSS[CFGGRIDRIGAQSGLGC]NSFRY-NH₂
(disulfide linkage between C7 and C23)

1. A polypeptide comprising:
(SEQ ID NO: 3)
X₁X₂X₃RSSCFX₉X₁₀X₁₁IX₁₃RIGX₁₇X₁₈SGLGCPSX₂₆RX₂₈X₂₉,
wherein:
X₁ is absent, S or E,
X₂ is absent, L, K, 4-Pal, H or E,
X₃ is absent, R, β-Ala, P, K, E or G,
X₉ is G, 4-Pal or H,
X₁₀ is G, K, R, or Dap,
X₁₁ is R, K, G or Dap,
X₁₃ is D or G,
X₁₇ is A, H, Dap, K, R or Orn,
X₁₈ is Q, Y or 4-Pal,
X₂₆ is F or L,
X₂₈ is Y, H or 4-Pal and
X₂₉ is either absent or selected from
GGP,
(SEQ ID NO: 4)
SGAPPPE,
(SEQ ID NO: 5)
KITAKEDE,
(SEQ ID NO: 6)
GPSSGAPPPE,
(SEQ ID NO: 7)
GPSSGAPPPS,
(SEQ ID NO: 8)
GGSSGAPPPS,
(SEQ ID NO: 9)
GGPSSGAPPPS,
(SEQ ID NO: 10)
KGPSSGAPPPS,
(SEQ ID NO: 11)
GGKSSGAPPPS,
(SEQ ID NO: 12)
GGPPS-Aib-KPPPK,
(SEQ ID NO: 13)
GSPSSGAPPPS,
(SEQ ID NO: 14)
RITAREDKQGYA,
(SEQ ID NO: 15)
RITAREDKQGEA,
(SEQ ID NO: 16)
GSPSSGAPPPS-PEG24-G,
(SEQ ID NO: 17)
SGSPSSGAPPPSG,
(SEQ ID NO: 18)
GGESSGEPPPSEE,
(SEQ ID NO: 19)
GSGSPSSGAPPPSG,
and
(SEQ ID NO: 20)
SGSPSSGAPPPSEEEG
and the C-terminal amino acid is optionally amidated.
or a pharmaceutically acceptable salt thereof.

2. The polypeptide of claim 1, or a pharmaceutically acceptable salt thereof, comprising a disulfide linkage or a thioacetal linkage between cysteine at position 7 and cysteine at position 23 of SEQ ID NO:3.

3. The polypeptide of claim 1 or 2, or a pharmaceutically acceptable salt thereof, further comprising a fatty acid conjugated to the amino acid present at the N terminus of the polypeptide, and comprising a structure:
fatty acid-Z1-Z2-Z3-X₁X₂X₃RSSCFX₉X₁₀
X₁₁IDRIGX₁₇X₁₈SGLGCX₂₄SX₂₆RX₂₈X₂₉,
wherein the fatty acid is a C₁₆-C₂₆ fatty acid and is conjugated to the amino acid present at the N terminus of the polypeptide through a structure Z1-Z2-Z3, wherein
Z1 is an amino acid selected from γGlu, E and β-Ala,
Z2 is selected from (EK)_{b}G, (EP)_{b}G, K(EK)_{c}G, (EK)_{c}E and APPSG (SEQ ID NO:24), wherein b is 2, 3 or 4 and c is 1, 2, 3 or 4, and
Z3 is either absent or is selected from (polyethylene glycol)ₘ wherein m is a whole number selected from 10 to 30 or ((2-[2-(2-amino-ethoxy)-ethoxy]-acetyl))ₙ wherein n is selected from 1 to 10.

4. The polypeptide of any one of claims 1 to 3, or a pharmaceutically acceptable salt thereof, wherein
X₁ is S or E,
X₂ is K or 4-Pal,
X₃ is R, β-Ala or K,
X₉ is G,
X₁₀ is G or K,
X₁₁ is R or K,
X₁₃ is D or G,
X₁₇ is H,
X₁₈ is Q or Y,
X₂₆ is F,
X₂₈ is H, and
X₂₉ is selected from GGPSSGAPPPS (SEQ ID NO:9), GGKSSGAPPPS (SEQ ID NO:11), and GSPSSGAPPPS (SEQ ID NO:13).

5. The polypeptide of claim 3 or 4, or a pharmaceutically acceptable salt thereof, wherein the fatty acid is a C₁₆-C₂₂ fatty acid.

6. The polypeptide of claim 5, or a pharmaceutically acceptable salt thereof, wherein the Z1 is γ-Glu.

7. The polypeptide of claim 6, or a pharmaceutically acceptable salt thereof, wherein the Z2 comprises a sequence selected from EKEKEKG (SEQ ID NO:22), EPEPEPG (SEQ ID NO:23) and APPSG (SEQ ID NO:24).

8. The polypeptide of claim 7, or a pharmaceutically acceptable salt thereof, wherein the Z3 is absent or selected from (polyethylene glycol)ₘ wherein m is 12 or 24, and ((2-[2-(2-amino-ethoxy)-ethoxy]-acetyl))ₙ wherein n is 4, 6 or 8.

9. The polypeptide of any one of claims 1 to 8 or a pharmaceutically acceptable salt thereof, wherein the polypeptide is selected from SEQ ID NO:28 to 167.

10. The polypeptide of any one of claims 1 to 9, or a pharmaceutically acceptable salt thereof, wherein the polypeptide is selected from SEQ ID NO:28, 45, 50, 51, 78, 83, 84, 97, 98, 144, 158 and 159.

11. The polypeptide of any one of claims 1 to 10 or a pharmaceutically acceptable salt thereof, wherein the C terminal is amidated.

12. The polypeptide of any one of claims 1 to 11, or a pharmaceutically acceptable salt thereof, wherein the polypeptide is an agonist of NPR-A.

13. A pharmaceutical composition comprising the polypeptide of any one of claims 1 to 12 or a pharmaceutically acceptable salt thereof, and at least one pharmaceutically acceptable carrier, diluent, or excipient.

14. The pharmaceutical composition of claim 13, wherein the composition is formulated for subcutaneous (SQ) or intravenous (IV) administration.

15. The pharmaceutical composition of claim 14, wherein the composition is formulated for SQ administration.

16. A method for treating a cardiovascular disease (CVD) comprising administering to a patient in need thereof, an effective amount of a polypeptide of any one of claims 1 to 12, or a pharmaceutically acceptable salt thereof, or the pharmaceutical composition of claim 13.

17. The method of claim 16, wherein the CVD is heart failure.

18. The method of claim 17, wherein the heart failure is HFpEF.

19-25. (canceled)
